(19) United States
(12) Patent Application Publication
(10) Pub. No.: US 2025/0019717 A1
(43) Pub. Date: Jan. 16, 2025

(54) DURABLE DOWNY MILDEW RESISTANCE IN SPINACH

(71) Applicant: KWS VEGETABLES B.V., GELDERLAND (NL)

(72) Inventors: JOHANNES SIMON GROENENDIJK, WAGENINGEN (NL); JAN DE VISSER, ANDIJK (NL); JOHANNES MARINUS RIJK, ENS (NL)

(21) Appl. No.: 18/294,306

(22) PCT Filed: Aug. 5, 2022
(86) PCT No.: PCT/EP2022/072119

§ 371 Date: Feb. 1, 2024

(30) Foreign Application Priority Data
Aug. 6, 2021 (EP) 21190145.9

(51) Int. Cl. C12N 15/82

(52) U.S. Cl. CPC C12N 15/8282

(57) ABSTRACT

The present disclosure relates to methods of identifying and selecting plants with resistance to downy mildew and further plant pathogens. Provided are methods to identify a novel gene that encode proteins associated with plant resistance to downy mildew and further plant pathogens, and uses thereof. This disease resistant gene is useful in the production of resistant plants through breeding, transgenic modification, or genome editing.

### SEQUENCE LISTING

The instant application contains a sequence listing that has been submitted in XML format by electronic submission and is hereby incorporated by reference in its entirety. Said XML copy, created on Sep. 3, 2024, is named “P14647US00_SequenceListing.xml” and is 1,110,283 bytes in size.

### TECHNICAL FIELD

The present disclosure relates to the field of plant breeding and molecular biology and more specifically to novel genes providing resistance to downy mildew and uses thereof.

### BACKGROUND

Spinach (Spinacia oleracea) is a flowering plant from the Amaranthaceae family that is grown as a vegetable. The consumable parts of spinach are the leaves from the vegetative stage. Spinach is sold loose, bunched, in prepacked bags, canned, or frozen. There are three basic types of spinach, namely the savoy, semi-savoy and smooth types. Savoy has crinkly and curly leaves. Flat or smooth leaf spinach has in general broad, smooth leaves. Semi-savoy is a variety with slightly crinkled leaves. The main market for spinach is baby-leaf. Baby spinach leaves are usually of the flat-leaf variety and usually the harvested leaves are not longer than about eight centimeters. These tender, sweet leaves are sold loose rather than in bunch. They are often used in salads, but can also be lightly cooked. Downy mildew, which in spinach is caused by the pathogen Peronospora effusa (also known as P. farinosa f. sp. spinaciae), is a major threat for spinach growers, because it affects the harvested plant parts, namely the leaves. Infection makes the leaves unsuitable for sale and consumption, as it manifests itself phenotypically as yellow lesions on the older leaves, and on the abaxial leaf surface a greyish fungal growth can be observed. The infection can spread very rapidly, and it can occur both in glasshouse cultivation, in vertical farming, and in soil cultivation. The optimal temperature for formation and germination of P. effusa is 9 to 12° C., and it is facilitated by a high relative humidity. When pathogens are deposited on a humid leaf surface they can readily germinate and infect the leaf. Pathogen growth is optimal between 8 and 20° C. and a relative humidity of ≥80%, and growth can be observed within 6 and 13 days after infection. P. effusa can survive in the soil for up to 3 years, or in seeds or living plants. Various plants or germplasms having at least partial resistance to downy mildew caused by P. effusa are known, for instance from Monsanto Vegetable IP Management (Spinacia Oleracea L., Kona-Swb2636 and Smbs151262-Smb-S015-1262m), from She Hongbing et al., 2018 (Fine mapping and candidate gene screening of the downy mildew resistance geneRPF1 in Spinach), and WO 2013/064436. Methods for modulating plant growth and phenotype are known in the art, for example from US 2017/037422. In addition, various spinach genes are known, as can be retrieved from the NCBI database. In recent years various resistance genes have been identified that provide spinach plants with a resistance against downy mildew. However, it has been observed that previously resistant spinach cultivars can again become susceptible to the pathogen. Investigations revealed that the cultivars themselves had not changed, and that the loss of downy mildew resistance must therefore be due to P. effusa overcoming the resistance in these spinach cultivars. The downy mildew races that were able to infect resistant spinach cultivars have been identified on a differential reference set, used to test spinach cultivars for resistance. The differential set comprises a series of spinach cultivars (hybrids) that have different resistance patterns to the currently identified pathogenic races. To date, 19 pathogenic races of spinach downy mildew (Pe) have been officially identified and characterized. Races 4 through 10 were identified between 1990 and 2009, which illustrates the versatility and adaptability of the pathogen to overcome resistances in spinach. In 2014, isolate UA1014APLP (also known as UA1014 and now Pe:17) was identified by the Correll lab of the University of Arkansas. The International Working Group Peronospora in Spinach (IWGP) has named two new races of P. effusa in spinach: Pe:18 and Pe:19. Both races pose a major threat to the spinach industry.

In different geographical regions different combinations of pathogenic races or isolates occur, and the spinach industry therefore has a strong demand for spinach cultivars that are resistant to as many relevant downy mildew races as possible, preferably to all races that may occur in their region, and even to the newest threats that cannot be countered with the resistances that are present in the commercially available spinach cultivars.

It is crucial to stay at the forefront of developments in this field, as Peronospora continuously develops the ability to break the resistances that are present in commercial spinach varieties. For this reason new resistance genes are very valuable assets, and they form an important research focus in spinach breeding. Due to the threat of not finding new R-genes to a newly developed race of the pathogen, there is also a need to find alternative resistances that are broad-spectrum (most/all races) and more durable.

The goal of spinach breeders is to rapidly develop spinach varieties with resistance to as many Peronospora races as possible, including the latest identified. To date, 19 Pe races are officially recognized and made publicly available from the Department of Plant Pathology, University of Arkansas, Fayetteville, AR 72701, USA, and also from NAK Tuinbouw, Sotaweg 22, 2371 GD Roelofarendsveen, the Netherlands. Recently, Pe. 20 has been identified in certain regions, and additional races of Peronospora effusa are expected to develop continuously.

### SUMMARY

Compositions and methods useful in identifying and selecting plant disease susceptibility genes, or “S-genes,” are provided herein. The compositions and methods are useful in generating resistant plants by chemical mutagenesis, selecting disease resistant plants, creating transgenic resistant plants, and/or creating resistant genome edited plants. Plants having newly conferred or enhanced resistance to various plant diseases as compared to control plants are also provided herein. In some embodiments, the compositions and methods are useful in generating resistant plants by chemical mutagenesis, selecting downy mildew (DM) disease resistant spinach plants, creating transgenic DM resistant spinach plants, and/or creating DM resistant genome edited spinach plants.

A DM resistant spinach plant may be crossed to a second spinach plant in order to obtain a progeny spinach plant that has the resistant gene allele. The disease resistance may be newly conferred or enhanced relative to a control plant that does not have the favorable allele. The DM resistant gene allele may be further refined to a chromosomal interval defined by and including defined markers. In some embodiments, the methods for identifying and/or selecting plants having resistance to DM are presented. In these methods, DNA of a spinach plant is analyzed for the presence of a resistant gene allele on chromosome 4 that is associated with DM resistance, wherein said resistant gene allele comprises a “T” at Spov3_chr4_93275081 (position 101 of reference sequence SEQ ID NO: 241), an “A” at Spov3_chr4_105049870 (position 101 of reference sequence SEQ ID NO: 242), an “A” at Spov3_chr4_107241402 (position 101 of reference sequence SEQ ID NO: 243), an “A” at Spov3_chr4_109546568 (position 101 of reference sequence SEQ ID NO: 244), an “A” at Spov3_chr4_109698808 (position 101 of reference sequence SEQ ID NO: 245), an “A” at Spov3_chr4_112008390 (position 101 of reference sequence SEQ ID NO: 246), a “T” at Spov3_chr4_112574123 (position 101 of reference sequence SEQ ID NO: 247), a “T” at Spov3_chr4_117783935 (position 101 of reference sequence SEQ ID NO: 248), an “A” at Spov3_chr4_118191085 (position 101 of reference sequence SEQ ID NO: 249), an “A” at Spov3_chr4_118788268 (position 101 of reference sequence SEQ ID NO: 250), or an “A” at Spov3_chr4_121142541 (position 101 of reference sequence SEQ ID NO: 251), and a plant is identified and/or selected as having DM resistance if said resistant gene allele is detected.

In some embodiments, the methods for identifying and/or selecting plants having resistance to DM comprise detecting or selecting a genomic region comprising SEQ ID NO: 1, SEQ ID NO: 93, or SEQ ID NO: 169. The DM resistance may be newly conferred or enhanced relative to a control plant that does not have the favorable allele. In an embodiment, the DM resistant region comprises a gene encoding a clathrin adaptor medium subunit protein that confers or enhances resistance to DM. In some embodiments, the clathrin adaptor medium subunit protein comprises the amino acid sequence as set forth in SEQ ID NO: 3. In an embodiment, the DM resistant region comprises a gene encoding a pentatricopeptide repeat-containing protein that confers or enhances resistance to DM. In some embodiments, the pentatricopeptide repeat-containing protein comprises the amino acid sequence as set forth in SEQ ID NO: 95. In an embodiment, the DM resistant region comprises a gene encoding an RPS2-like resistance protein that confers or enhances resistance to DM. In some embodiments, the RPS2-like resistance protein comprises the amino acid sequence as set forth in SEQ ID NO: 171.

In another embodiment, methods of identifying and/or selecting plants with DM resistance are provided in which one or more marker alleles linked to and associated with any of: a “T” at Spov3_chr4_93275081 (position 101 of reference sequence SEQ ID NO: 241), an “A” at Spov3_chr4_105049870 (position 101 of reference sequence SEQ ID NO: 242), an “A” at Spov3_chr4_107241402 (position 101 of reference sequence SEQ ID NO: 243), an “A” at Spov3_chr4_109546568 (position 101 of reference sequence SEQ ID NO: 244), an “A” at Spov3_chr4_109698808 (position 101 of reference sequence SEQ ID NO: 245), an “A” at Spov3_chr4_112008390 (position 101 of reference sequence SEQ ID NO: 246), a “T” at Spov3_chr4_112574123 (position 101 of reference sequence SEQ ID NO: 247), a “T” at Spov3_chr4_117783935 (position 101 of reference sequence SEQ ID NO: 248), an “A” at Spov3_chr4_118191085 (position 101 of reference sequence SEQ ID NO: 249), an “A” at Spov3_chr4_118788268 (position 101 of reference sequence SEQ ID NO: 250), or an “A” at Spov3_chr4_121142541 (position 101 of reference sequence SEQ ID NO: 251), are detected in a plant, and a plant having the one or more marker alleles is selected. The one or more marker alleles may be linked by 10 cM, 9 CM, 8 CM, 7 CM, 6 CM, 5 CM, 4 CM, 3 CM, 2 cM, 1 CM, 0.9 CM, 0.8 CM, 0.7 CM, 0.6 CM, 0.5 CM, 0.4 CM, 0.3 cM, 0.2 cM, or 0.1 cM or less on a single meiosis based genetic map. The selected plant may be crossed to a second plant to obtain a progeny plant that has one or more marker alleles linked to and associated with any of a “T” at Spov3_chr4_93275081 (position 101 of reference sequence SEQ ID NO: 241), an “A” at Spov3_chr4_105049870 (position 101 of reference sequence SEQ ID NO: 242), an “A” at Spov3_chr4_107241402 (position 101 of reference sequence SEQ ID NO: 243), an “A” at Spov3_chr4_109546568 (position 101 of reference sequence SEQ ID NO: 244), an “A” at Spov3_chr4_109698808 (position 101 of reference sequence SEQ ID NO: 245), an “A” at Spov3_chr4_112008390 (position 101 of reference sequence SEQ ID NO: 246), a “T” at Spov3_chr4_112574123 (position 101 of reference sequence SEQ ID NO: 247), a “T” at Spov3_chr4_117783935 (position 101 of reference sequence SEQ ID NO: 248), an “A” at Spov3_chr4_118191085 (position 101 of reference sequence SEQ ID NO: 249), an “A” at Spov3_chr4_118788268 (position 15 101 of reference sequence SEQ ID NO: 250), or an “A” at Spov3_chr4_121142541 (position 101 of reference sequence SEQ ID NO: 251).

In another embodiment, methods of introgressing a gene allele associated with DM resistance are presented herein. In these methods, a population of plants is screened with one or more markers to determine if any of the plants has a gene allele associated with DM resistance, and at least one plant that has the gene allele associated with DM resistance is selected from the population. The gene allele comprises a “T” at Spov3_chr4_93275081 (position 101 of reference sequence SEQ ID NO: 241), an “A” at Spov3_chr4_105049870 (position 101 of reference sequence SEQ ID NO: 242), an “A” at Spov3_chr4_107241402 (position 101 of reference sequence SEQ ID NO: 243), an “A” at Spov3_chr4_109546568 (position 101 of reference sequence SEQ ID NO: 244), an “A” at Spov3_chr4_109698808 (position 101 of reference sequence SEQ ID NO: 245), an “A” at Spov3_chr4_112008390 (position 101 of reference sequence SEQ ID NO: 246), a “T” at Spov3_chr4_112574123 (position 101 of reference sequence SEQ ID NO: 247), a “T” at Spov3_chr4_117783935 (position 101 of reference sequence SEQ ID NO: 248), an “A” at Spov3_chr4_118191085 (position 101 of reference sequence SEQ ID NO: 249), an “A” at Spov3_chr4_118788268 (position 101 of reference sequence SEQ ID NO: 250), or an “A” at Spov3_chr4_121142541 (position 101 of reference sequence SEQ ID NO: 251).

In some embodiments, introduction of DM resistant genes from resistant to susceptible lines may be achieved either by marker-assisted trait introgression, transgenic, or genome editing (including gene replacement or allele replacement) approaches.

The methods embodied by the present disclosure relate to a method for transforming a host cell, including a plant cell, comprising transforming the host cell with the polynucleotide of an embodiment of the present disclosure; a method for producing a plant comprising transforming a plant cell with the recombinant DNA construct of an embodiment of the present disclosure and regenerating a plant from the transformed plant cell, and methods of conferring or enhancing disease resistance, comprising transforming a plant with the recombinant DNA construct disclosed herein.

In one embodiment a method of altering the level of expression of a protein capable of conferring disease resistance in a plant or plant cell is provided wherein the method comprises transforming a plant cell with a recombinant DNA construct disclosed herein and growing the transformed plant cell under conditions that are suitable for expression of the recombinant DNA construct wherein expression of the recombinant DNA construct results in production of altered levels of a protein capable of conferring disease resistance in the transformed host.

Provided herein is a method of modifying plant disease resistance, the method comprising introducing one or more nucleotide modifications through a targeted DNA break at a genomic locus of a plant, wherein the genomic locus comprises an S-gene involved in disease resistance encoding a clathrin adaptor medium subunit protein, a pentatricopeptide repeat-containing family protein, or an RPS2-like resistance protein, and wherein the plant disease resistance is modified compared to a control plant not comprising the one or more introduced genetic modifications. In certain embodiments, the S-gene comprise a polynucleotide that is at least 80%, at least 85%, at least 90%, at least 95%, at least 96%, at least 97%, at least 98%, or at least 99% identical to a sequence selected from SEQ ID NOs: 4, 7-34, 96, 99-121, 172, 175-196, 252-270, 316-320, and 331 or encodes a polypeptide comprising an amino acid sequence that is at least 80%, at least 85%, at least 90%, at least 95%, at least 96%, at least 97%, at least 98%, or at least 99% identical to a sequence selected from SEQ ID NOs: 6, 64-92, 98, 145-168, 174, 219-240, 294-315, 326-330, and 333. In certain embodiments, the targeted DNA modification targets more than one distinct genomic loci that is involved in disease resistance of the plant.

In certain embodiments, the targeted DNA modification is selected from the group consisting of insertion, deletion, single nucleotide polymorphism (SNP), and a polynucleotide modification, such that the expression of the polypeptide encoded by the S-gene is altered (increased or reduced). In certain embodiments, the targeted DNA modification results in one or more of the following: altered (increased or reduced) expression of the S-gene; generation of one or more alternative spliced transcripts of the S-gene; alteration such as deletion of one or more DNA binding domains; frameshift mutation in one or more exons of the S-gene; alteration such as deletion of a substantial portion of the S-gene or deletion of the full-length open reading frame of the S-gene; induction or repression of an enhancer motif present within a regulatory region encoding the S-gene; modification of one or more nucleotides or alteration such as deletion of a regulatory element operably linked to the expression of the S-gene, wherein the regulatory element is present within a promoter, intron, 3′UTR, terminator or a combination thereof. In certain embodiments, the targeted DNA modification targets the genomic locus of the S-gene such that the one or more nucleotide modifications are present within (a) the same coding region; (b) non-coding region; (c) regulatory sequence; (d) untranslated region, or (e) any combination of (a)-(d) of an endogenous polynucleotide encoding a polypeptide that is involved in disease resistance and critically facilitates compatibility.

In certain embodiments, the targeted DNA modification is introduced by an RNA-guided endonuclease, a site-specific deaminase, or a site-specific endonuclease. In certain embodiments, the targeted DNA modification is through a genome modification technique selected from the group consisting of polynucleotide-guided endonuclease, CRISPR-Cas endonucleases, base editing deaminases, zinc finger nuclease, a transcription activator-like effector nuclease (TALEN), engineered site-specific meganucleases, or Argonaute. In certain embodiments, the targeted DNA modification is induced by using a guide RNA that corresponds to a target sequence comprising a polynucleotide that is at least 80%, at least 85%, at least 90%, at least 95%, at least 96%, at least 97%, at least 98%, or at least 99% identical to a sequence selected from SEQ ID NOs: 4, 7-34, 96, 99-121, 172, 175-196, 252-270, 316-320, and 331 or a polynucleotide that encodes a polypeptide comprising an amino acid sequence that is at least 80%, at least 85%, at least 90%, at least 95%, at least 96%, at least 97%, at least 98%, or at least 99% identical to a sequence selected from SEQ ID NOs: 6, 64-92, 98, 145-168, 174, 219-240, 294-315, 326-330, and 333. In certain embodiments, the plant exhibits increased disease resistance when the targeted DNA modification results in altered expression or activity of the protein encoded by the S-gene. In certain embodiments, the plant is a spinach plant. In certain embodiments, the disease is downy mildew.

Also provided herein is a plant exhibiting increased disease resistance comprising a modified genomic locus comprising an S-gene involved in disease resistance, wherein the genomic locus comprises one or more introduced mutations compared to a control plant and wherein the genomic locus comprises a polynucleotide that is at least 80%, at least 85%, at least 90%, at least 95%, at least 96%, at least 97%, at least 98%, or at least 99% identical to a sequence selected from SEQ ID NOs: 4, 7-34, 96, 99-121, 172, 175-196, 252-270, 316-320, and 331 or encodes a polypeptide that is at least 80%, at least 85%, at least 90%, at least 95%, at least 96%, at least 97%, at least 98%, or at least 99% identical to the amino acid sequence selected from SEQ ID NOs: 6, 64-92, 98, 145-168, 174, 219-240, 294-315, 326-330, and 333.

Also provided herein is a recombinant DNA construct comprising a polynucleotide sequence that is at least 80%, at least 85%, at least 90%, at least 95%, at least 96%, at least 97%, at least 98%, or at least 99% identical to a sequence selected from SEQ ID NOs: 4, 7-34, 96, 99-121, 172, 175-196, 252-270, 316-320, and 331 or a polynucleotide encoding an amino acid sequence that is at least 80%, at least 85%, at least 90%, at least 95%, at least 96%, at least 97%, at least 98%, or at least 99% identical to an amino acid sequence selected from SEQ ID NOs: 6, 64-92, 98, 145-168, 174, 219-240, 294-315, 326-330, and 333, operably linked to at least one heterologous nucleic acid sequence.

Additionally, provided herein is a plant cell comprising a recombinant DNA construct comprising a polynucleotide sequence that is at least 80%, at least 85%, at least 90%, at least 95%, at least 96%, at least 97%, at least 98%, or at least 99% identical to a sequence selected from SEQ ID NOs 4, 7-34, 96, 99-121, 172, 175-196, 252-270, 316-320, and 331 or a polynucleotide encoding an amino acid sequence that is at least 80%, at least 85%, at least 90%, at least 95%, at least 96%, at least 97%, at least 98%, or at least 99% identical to an amino acid sequence selected from SEQ ID NOs: 6, 64-92, 98, 145-168, 174, 219-240, 294-315, 326-330, and 333, operably linked to at least one heterologous nucleic acid sequence.

Provided herein is a guide RNA sequence that targets a genomic locus of a plant cell, wherein the genomic locus comprises a polynucleotide that is at least 80%, at least 85%, at least 90%, at least 95%, at least 96%, at least 97%, at least 98%, or at least 99% identical to a sequence selected from SEQ ID NOs: 4, 7-34, 96, 99-121, 172, 175-196, 252-270, 316-320, and 331 or a polynucleotide that encodes a polypeptide comprising an amino acid sequence that is at least 80%, at least 85%, at least 90%, at least 95%, at least 96%, at least 97%, at least 98%, or at least 99% identical to a sequence selected from SEQ ID NOs: 6, 64-92, 98, 145-168, 174, 219-240, 294-315, 326-330, and 333. In certain embodiments, the guide RNA is present in a recombinant DNA construct.

While multiple embodiments are disclosed, still other embodiments of the inventions will become apparent to those skilled in the art from the following detailed description, which shows and describes illustrative embodiments of the invention. Accordingly, the figures and detailed description are to be regarded as illustrative in nature and not restrictive.

### BRIEF DESCRIPTION OF THE FIGURES

FIG. 1 shows SNP alleles informative for EMS transitions as a percentage of the EMS alleles in the total read coverage. Drawn are the percentages (Y-axis: % R and % S) at their positions on Chr4 in Phytozome: Soleracea_575_Spov3 mentioned in Examples (X-axis: 0-122 Mb).

FIG. 2 shows GWAS analysis of 91 EMS-induced SNPs and susceptibility/resistance scores to Pe:16. Shown is a Manhattan plot of significant scores of SNPs and their positions on spinach Chr1 to Chr6 in Phytozome: Soleracea_575_Spov3 mentioned in Examples. There is a highly significant correlation of 11 SNPs at the distal end of Chr4.

FIG. 3 shows a GWAS analysis of B11-509 specific SNPs and susceptibility/resistance scores to Pe:16. Shown is a Manhattan plot of significant scores of SNPs in the region 112-118 Mb of Chr4. There is a clear correlation of 3 SNPs in 0.62 Mb region surrounding Spov3_chr4.04649 (RPS2-Like).

FIG. 4 shows a sequence Alignment of reassembled contig_59494 mutant B11-509 EMS22-20 and Gene Model SOV1g044450_coding_sequence of a new Viroflay high quality assembly. Identical residues are shown as ‘*’, whereas different residues are shown as a space: intron1 (13-290 bp), G>A (296), G>C (1751), −/T (1755), deletion (1756-1946), −/G (1947) and G>A (2120). T>C (1177) and T>A (12007) are synonymous mutations.

FIG. 5 shows a sequence Alignment of wild type Viroflay Gene Model SOV19044450_protein and the translated ortholog sequence of mutant B11-509 EMS22-20. Identical residues are shown as ‘*’, whereas different residues are shown as a space: K490N, L493C and G614R.

FIG. 6 shows a differential expression analysis of Soleracea_575_Spov3 Gene Model Chr4.04649 (RPS2-like). Shown are relative expression levels for wild type B11-509 and mutant EMS22-20 at 4 time points in untreated (M, Mock) and Pe:16 infected (I) samples. The expression is upregulated in mutant plants compared to the wild-type B11-509 plants at all time points in the mock-treatment (uninfected plants) and at three time points in Pe:16-infected plants, indicating an overall increased gene expression of RPS2-like gene in the EMS22-20 plants, irrespective of the received treatment (mock or Pe:16).

FIG. 7 shows the infection process of DM isolate Pe:16 on leaves of both B11-509 and EMS22-20 using Trypan Blue staining and microscopical analysis. Shown is that the infection process in B11-509, i.e. penetration of the leaves by the appressoria, and hyphae growth is clearly visible. In addition, it is believed that haustoria are formed in B11-509. In contrast, penetration of hyphae is not visible in mutant EMS22-20.

FIG. 8 shows a microscopical analysis of the infection process of DM isolate Pe:16 in mutant EMS22-20, at day 4 and day 6 (lower right). Shown is that spores and hyphae cluster together on the leaf surface and are often washed off from the leaf.

### DETAILED DESCRIPTION

So that the present invention may be more readily understood, certain terms are first defined. Unless defined otherwise, all technical and scientific terms used herein have the same meaning as commonly understood by one of ordinary skill in the art to which embodiments of the invention pertain. Many methods and materials similar, modified, or equivalent to those described herein can be used in the practice of the embodiments of the present invention without undue experimentation, the preferred materials and methods are described herein. In describing and claiming the embodiments of the present invention, the following terminology will be used in accordance with the definitions set out below.

It is to be understood that all terminology used herein is for the purpose of describing particular embodiments only, and is not intended to be limiting in any manner or scope. For example, as used in this specification and the appended claims, the singular forms “a,” “an” and “the” can include plural referents unless the content clearly indicates otherwise. Similarly, the word “or” is intended to include “and” unless the context clearly indicate otherwise. The word “or” means any one member of a particular list and also includes any combination of members of that list. Further, all units, prefixes, and symbols may be denoted in its SI accepted form.

Numeric ranges recited within the specification are inclusive of the numbers defining the range and include each integer within the defined range. Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, 1½, and 4¾. This applies regardless of the breadth of the range.

The term “about,” as used herein, refers to variation in the numerical quantity that can occur, for example, through typical measuring techniques and equipment, with respect to any quantifiable variable, including, but not limited to, mass, volume, time, and temperature. Further, given solid and liquid handling procedures used in the real world, there is certain inadvertent error and variation that is likely through differences in the manufacture, source, or purity of the ingredients used to make the compositions or carry out the methods and the like. The term “about” also encompasses these variations. Whether or not modified by the term “about,” the claims include equivalents to the quantities.

An allele is “associated with” a trait when it is part of or linked to a DNA sequence, or is an allele that affects the expression of the trait. The presence of the allele is an indicator of how the trait will be expressed.

As used herein, the term “chromosomal interval” designates a contiguous linear span of genomic DNA that resides in planta on a single chromosome. The genetic elements or genes located on a single chromosomal interval are physically linked. The size of a chromosomal interval is not particularly limited. In some aspects, the genetic elements located within a single chromosomal interval are genetically linked, typically with a genetic recombination distance of, for example, less than or equal to 20 cM, or alternatively, less than or equal to 10 cM. That is, two genetic elements within a single chromosomal interval undergo recombination at a frequency of less than or equal to 20% or 10%.

The phrase “closely linked”, in the present application, means that recombination between two linked loci occurs with a frequency of equal to or less than about 10% (i.e., are separated on a genetic map by not more than 10 cM). Put another way, the closely linked loci co-segregate at least 90% of the time. Marker loci are especially useful with respect to the subject matter of the current disclosure when they demonstrate a significant probability of co segregation (linkage) with a desired trait (e.g., resistance to southern corn rust). Closely linked loci such as a marker locus and a second locus can display an inter-locus recombination frequency of 10% or less, preferably about 9% or less, still more preferably about 8% or less, yet more preferably about 7% or less, still more preferably about 6% or less, yet more preferably about 5% or less, still more preferably about 4% or less, yet more preferably about 3% or less, and still more preferably about 2% or less. In highly preferred embodiments, the relevant loci display a recombination a frequency of about 1% or less, e.g., about 0.75% or less, more preferably about 0.5% or less, or yet more preferably about 0.25% or less. Two loci that are localized to the same chromosome, and at such a distance that recombination between the two loci occurs at a frequency of less than 10% (e.g., about 9%, 8%, 7%, 6%, 5%, 4%, 3%, 2%, 1%, 0.75%, 0.5%, 0.25%, or less) are also said to be “proximal to” each other. In some cases, two different markers can have the same genetic map coordinates. In that case, the two markers are in such close proximity to each other that recombination occurs between them with such low frequency that it is undetectable.

The term “crossed” or “cross” refers to a sexual cross and involved the fusion of two haploid gametes via pollination to produce diploid progeny (e.g., cells, seeds or plants). The term encompasses both the pollination of one plant by another and selfing (or self-pollination, e.g., when the pollen and ovule are from the same plant).

As used to herein, “disease resistant” or “have resistance to a disease” refers to a plant showing increase resistance to a disease compared to a control plant. Disease resistance may manifest in fewer and/or smaller lesions, increased plant health, increased yield, increased root mass, increased plant vigor, less or no discoloration, increased growth, reduced necrotic area, or reduced wilting. In some embodiments, an allele may show resistance to one or more diseases.

Diseases affecting spinach plants include, but are not limited to, anthracnose (Colletotrichum dematium f. sp. spinaciae), damping off/seedling blight (Pythium ultimum), downy mildew (Peronospora effusa), fusarium wilt (Fusarium oxysporum f. sp. spinaciae), Stemphylium leaf spot (Stemphylium versicarium, Stemphylium beticola, Stemphylium drummondii), verticillium wilt (Verticillium dahlia), white rust (Albugo occidentalis), black root rot (Aphanomyces cochlioides), and Cladosporium leaf spot (Cladosporium variabile). Pathogens causing said diseases are thus widespread over various phyla. For example, Peronospora species are part of the phylum Oomycota, while Colletotrichum species are part of the phylum Ascomyceta.

Diseases affecting basil plants include, but are not limited to, downy mildew (Peronospora belbahrii).

Diseases affecting common bean plants include, but are not limited to, anthracnose (Colletotrichum lindemuthianum), pythium root rot (Pythium ultimum), and white mold (Sclerotinia sclerotiorum).

Diseases affecting beet plants include, but are not limited to, black wood vessel (Pythium irregulare), damping off/black root/seedling blight (Pythium ultimum), downy mildew (Peronospora schachtii), and phytophthora wet rot (Phytophthora drechsleri).

Diseases affecting carrot plants include, but are not limited to, cavity spot (Pythium ssp.), lateral root dieback (Pythium ssp.), leaf blight (Alternaria dauci), powdery mildew (Erysiphe heraclei), and white mold (Sclerotinia sclerotiorum).

Diseases affecting plants in the family Cucurbitaceae (e.g., cucumber, melon, watermelon, squash, pumpkin, gourd, and others crops) include, but are not limited to, alternaria leaf spot (Alternaria cucumerina), anthracnose (Colletotrichum obiculare), downy mildew (Pseudoperonospora cubensis), gummy stem blight (Didymella bryoniae), and powdery mildew (Erysiphe cichoracearum, Sphaerotheca fuliginea).

Diseases affecting grape plants include, but are not limited to, downy mildew (Plasmopara viticola).

Diseases affecting hop plants include, but are not limited to, downy mildew (Pseudoperonospora humuli).

Diseases affecting lettuce plants include, but are not limited to, downy mildew (Bremia lactucae).

Diseases affecting plants in the family Solanaceae (e.g., eggplant, pepper, petunia, potato, tomato, tobacco) include, but are not limited to, powdery mildew (Leveillula taurica), Phytophthora blight (Phytophthora capsica), late blight (Phytophthora infestans), and blue mold (Peronospora hyoscyami f.sp. tabacina).

Diseases affecting rapeseed plants include, but are not limited to, blackleg (Leptosphaeria maculan), damping off (Rhizoctonia solani), and downy mildew (Peronospora parasitica).

Diseases affecting soybean plants include, but are not limited to, downy mildew (Peronospora manshurica).

Diseases affecting sunflower plants include, but are not limited to, downy mildew (Plasmopara halstedii).

Diseases affecting maize plants include, but are not limited to, brown stripe downy mildew (Scleropthora rayssiae var. zeae), crazy top (Sclerophthora macrospora), downy mildew (Peronosclerospora sorghi), graminicola downy mildew (Sclerospora graminicola), java downy mildew (Peronospora maydis), and philippine downy mildew (Peronospora philippinensis).

Diseases affecting sorghum plants include, but are not limited to, downy mildew (Peronosclerospora sorghi).

A plant having disease resistance may have 5, 10, 15, 20, 25, 30, 35, 40, 45, 50, 55, 60, 65, 70, 75, 80, 85, 90, 95, or 100% increased resistance to a disease compared to a control plant. In some embodiments, a plant may have 5, 10, 15, 20, 25, 30, 35, 40, 45, 50, 55, 60, 65, 70, 75, 80, 85, 90, 95, or 100% increased plant health in the presence of a disease compared to a control plant.

An “elite line” is any line that has resulted from breeding and selection for superior agronomic performance.

The term “expression”, as used herein, generally refers to the production of a functional end-product e.g., an mRNA or a protein (precursor or mature).

A “favorable allele” is the allele at a particular locus (a marker, a QTL, a gene etc.) that confers, or contributes to, an agronomically desirable phenotype, e.g., disease resistance, and that allows the identification of plants with that agronomically desirable phenotype. A favorable allele of a marker is a marker allele that segregates with the favorable phenotype.

As used herein, “gene” includes a nucleic acid fragment that expresses a functional molecule such as, but not limited to, a specific protein coding sequence and regulatory elements, such as those preceding (5′ non-coding sequences) and following (3′ non-coding sequences) the coding sequence.

“Genetic markers” are nucleic acids that are polymorphic in a population and where the alleles of which can be detected and distinguished by one or more analytic methods, e.g., RFLP, AFLP, isozyme, SNP, SSR, and the like. The term also refers to nucleic acid sequences complementary to the genomic sequences, such as nucleic acids used as probes. Markers corresponding to genetic polymorphisms between members of a population can be detected by methods well-established in the art. These include, e.g., PCR-based sequence specific amplification methods, detection of restriction fragment length polymorphisms (RFLP), detection of isozyme markers, detection of polynucleotide polymorphisms by allele specific hybridization (ASH), detection of amplified variable sequences of the plant genome, detection of self-sustained sequence replication, detection of simple sequence repeats (SSRs), detection of single nucleotide polymorphisms (SNPs), or detection of amplified fragment length polymorphisms (AFLPs). Well established methods are also known for the detection of expressed sequence tags (ESTs) and SSR markers derived from EST sequences and randomly amplified polymorphic DNA (RAPD).

“Germplasm” refers to genetic material of or from an individual (e.g., a plant), a group of individuals (e.g., a plant line, variety or family), or a clone derived from a line, variety, species, or culture, or more generally, all individuals within a species or for several species (e.g., maize germplasm collection or Andean germplasm collection). The germplasm can be part of an organism or cell, or can be separate from the organism or cell. In general, germplasm provides genetic material with a specific molecular makeup that provides a physical foundation for some or all of the hereditary qualities of an organism or cell culture. As used herein, germplasm includes cells, seed or tissues from which new plants may be grown, or plant parts, such as leaves, stems, pollen, or cells, that can be cultured into a whole plant.

A “genomic locus of a plant” as used herein, generally refers to the location on a chromosome of the plant where a gene, such as a polynucleotide involved in disease resistance, is found.

The term “homologous” refers to nucleic acid sequences that are derived from a common ancestral gene through natural or artificial processes (e.g., are members of the same gene family) and thus, typically, share sequence similarity. Typically, homologous nucleic acids have sufficient sequence identity that one of the sequences or its complement is able to selectively hybridize to the other under selective hybridization conditions. The term “selectively hybridizes” includes reference to hybridization, under stringent hybridization conditions, of a nucleic acid sequence to a specified nucleic acid target sequence to a detectably greater degree (e.g., at least 2-fold over background) than its hybridization to non-target nucleic acid sequences and to the substantial exclusion of non-target nucleic acids. Selectively hybridizing sequences have about at least 80% sequence identity, often at least 90% sequence identity and may have 95%, 97%, 99% or 100% sequence identity with each other. A nucleic acid that exhibits at least some degree of homology to a reference nucleic acid can be unique or identical to the reference nucleic acid or its complementary sequence.

The term “introduced” means providing a nucleic acid (e.g., expression construct) or protein into a cell. Introduced includes reference to the incorporation of a nucleic acid into a eukaryotic or prokaryotic cell where the nucleic acid may be incorporated into the genome of the cell, and includes reference to the transient provision of a nucleic acid or protein to the cell. Introduced includes reference to stable or transient transformation methods, as well as sexually crossing. Thus, “introduced” in the context of inserting a nucleic acid fragment (e.g., a recombinant DNA construct/expression construct) into a cell, means “transfection” or “transformation” or “transduction” and includes reference to the incorporation of a nucleic acid fragment into a eukaryotic or prokaryotic cell where the nucleic acid fragment may be incorporated into the genome of the cell (e.g., chromosome, plasmid, plastid or mitochondrial DNA), converted into an autonomous replicon, or transiently expressed (e.g., transfected mRNA).

The term “introgression” refers to the transmission of a desired allele of a genetic locus from one genetic background to another. For example, introgression of a desired allele at a specified locus can be transmitted to at least one progeny via a sexual cross between two parents of the same species, where at least one of the parents has the desired allele in its genome. Alternatively, for example, transmission of an allele can occur by recombination between two donor genomes, e.g., in a fused protoplast, where at least one of the donor protoplasts has the desired allele in its genome. The desired allele can be, e.g., detected by a marker that is associated with a phenotype, at a QTL, a transgene, or the like. Offspring comprising the desired allele may be repeatedly backcrossed to a line having a desired genetic background and selected for the desired allele, to result in the allele becoming fixed in a selected genetic background. The process of “introgressing” is often referred to as “backcrossing” when the process is repeated two or more times.

“Introgression fragment” or “introgression segment” or “introgression region” refers to a chromosome fragment (or chromosome part or region) which has been introduced into another plant of the same or related species by crossing or traditional breeding techniques, such as backcrossing, i.e. the introgressed fragment is the result of breeding methods referred to by the verb “to introgress” (such as backcrossing). It is understood that the term “introgression fragment” never includes a whole chromosome, but only a part of a chromosome. The introgression fragment can be large, e.g. even half of a chromosome, but is preferably smaller, such as about 15 Mb or less, such as about 10 Mb or less, about 9 Mb or less, about 8 Mb or less, about 7 Mb or less, about 6 Mb or less, about 5 Mb or less, about 4 Mb or less, about 3 Mb or less, about 2 Mb or less, about 1 Mb (equals 1,000,000 base pairs) or less, or about 0.5 Mb (equals 500,000 base pairs) or less, such as about 200,000 bp (equals 200 kilo base pairs) or less, about 100,000 bp (100 kb) or less, about 50,000 bp (50 kb) or less, about 25,000 bp (25 kb) or less.

A “line” or “strain” is a group of individuals of identical parentage that are generally inbred to some degree and that are generally homozygous and homogeneous at most loci (isogenic or near isogenic). A “subline” refers to an inbred subset of descendants that are genetically distinct from other similarly inbred subsets descended from the same progenitor.

As used herein, the term “linkage” is used to describe the degree with which one marker locus is associated with another marker locus or some other locus. The linkage relationship between a molecular marker and a locus affecting a phenotype is given as a “probability” or “adjusted probability”. Linkage can be expressed as a desired limit or range. For example, in some embodiments, any marker is linked (genetically and physically) to any other marker when the markers are separated by less than 50, 40, 30, 25, 20, or 15 map units (or cM) of a single meiosis map (a genetic map based on a population that has undergone one round of meiosis, such as e.g. an F2; the IB M2 maps consist of multiple meiosis). In some aspects, it is advantageous to define a bracketed range of linkage, for example, between 10 and 20 cM, between 10 and 30 cM, or between 10 and 40 cM. The more closely a marker is linked to a second locus, the better an indicator for the second locus that marker becomes. Thus, “closely linked loci” such as a marker locus and a second locus display an inter-locus recombination frequency of 10% or less, preferably about 9% or less, still more preferably about 8% or less, yet more preferably about 7% or less, still more preferably about 6% or less, yet more preferably about 5% or less, still more preferably about 4% or less, yet more preferably about 3% or less, and still more preferably about 2% or less. In highly preferred embodiments, the relevant loci display a recombination frequency of about 1% or less, e.g., about 0.75% or less, more preferably about 0.5% or less, or yet more preferably about 0.25% or less. Two loci that are localized to the same chromosome, and at such a distance that recombination between the two loci occurs at a frequency of less than 10% (e.g., about 9%, 8%, 7%, 6%, 5%, 4%, 3%, 2%, 1%, 0.75%, 0.5%, 0.25%, or less) are also said to be “in proximity to” each other. Since one cM is the distance between two markers that show a 1% recombination frequency, any marker is closely linked (genetically and physically) to any other marker that is in close proximity, e.g., at or less than 10 cM distant. Two closely linked markers on the same chromosome can be positioned 9, 8, 7, 6, 5, 4, 3, 2, 1, 0.75, 0.5 or 0.25 cM or less from each other.

A “locus” is a position on a chromosome, e.g. where a nucleotide, gene, sequence, or marker is located.

A “marker” is a means of finding a position on a genetic or physical map, or else linkages among markers and trait loci (loci affecting traits). The position that the marker detects may be known via detection of polymorphic alleles and their genetic mapping, or else by hybridization, sequence match or amplification of a sequence that has been physically mapped. A marker can be a DNA marker (detects DNA polymorphisms), a protein (detects variation at an encoded polypeptide), or a simply inherited phenotype. A DNA marker can be developed from genomic nucleotide sequence or from expressed nucleotide sequences (e.g., from a spliced RNA or a cDNA). Depending on the DNA marker technology, the marker may consist of complementary primers flanking the locus and/or complementary probes that hybridize to polymorphic alleles at the locus. A DNA marker, or a genetic marker, may also be used to describe the gene, DNA sequence or nucleotide on the chromosome itself (rather than the components used to detect the gene or DNA sequence) and is often used when that DNA marker is associated with a particular trait in human genetics (e.g. a marker for breast cancer). The term marker locus is the locus (gene, sequence or nucleotide) that the marker detects.

Markers can be defined by the type of polymorphism that they detect and also the marker technology used to detect the polymorphism. Marker types include but are not limited to, e.g., detection of restriction fragment length polymorphisms (RFLP), detection of isozyme markers, randomly amplified polymorphic DNA (RAPD), amplified fragment length polymorphisms (AFLPs), detection of simple sequence repeats (SSRs), detection of amplified variable sequences of the plant genome, detection of self-sustained sequence replication, or detection of single nucleotide polymorphisms (SNPs). SNPs can be detected e.g. via DNA sequencing, PCR-based sequence specific amplification methods, detection of polynucleotide polymorphisms by allele specific hybridization (ASH), dynamic allele-specific hybridization (DASH), molecular beacons, microarray hybridization, oligonucleotide ligase assays, Flap endonucleases, 5′ endonucleases, primer extension, single strand conformation polymorphism (SSCP) or temperature gradient gel electrophoresis (TGGE). DNA sequencing, such as the pyrosequencing technology has the advantage of being able to detect a series of linked SNP alleles that constitute a haplotype. Haplotypes tend to be more informative (detect a higher level of polymorphism) than SNPs.

A “marker allele”, alternatively an “allele of a marker locus”, can refer to one of a plurality of polymorphic nucleotide sequences found at a marker locus in a population.

“Marker assisted selection” (or MAS) is a process by which individual plants are selected based on marker genotypes. “Marker assisted counter-selection” is a process by which marker genotypes are used to identify plants that will not be selected, allowing them to be removed from a breeding program or planting.

A “marker locus” is a specific chromosome location in the genome of a species where a specific marker can be found. A marker locus can be used to track the presence of a second linked locus, e.g., one that affects the expression of a phenotypic trait. For example, a marker locus can be used to monitor segregation of alleles at a genetically or physically linked locus.

A “modified nucleotide” or “edited nucleotide” refers to a nucleotide sequence of interest that comprises at least one alteration when compared to its non-modified nucleotide sequence. Such “alterations” include, for example: (i) replacement of at least one nucleotide, (ii) a deletion of at least one nucleotide, (iii) an insertion of at least one nucleotide, or (iv) any combination of (i)-(iii).

The term “molecular marker” may be used to refer to a genetic marker, as defined above, or an encoded product thereof (e.g., a protein) used as a point of reference when identifying a linked locus. A marker can be derived from genomic nucleotide sequences or from expressed nucleotide sequences (e.g., from a spliced RNA, a cDNA, etc.), or from an encoded polypeptide. The term also refers to nucleic acid sequences complementary to or flanking the marker sequences, such as nucleic acids used as probes or primer pairs capable of amplifying the marker sequence. A “molecular marker probe” is a nucleic acid sequence or molecule that can be used to identify the presence of a marker locus, e.g., a nucleic acid probe that is complementary to a marker locus sequence. Alternatively, in some aspects, a marker probe refers to a probe of any type that is able to distinguish (i.e., genotype) the particular allele that is present at a marker locus. Nucleic acids are “complementary” when they specifically hybridize in solution. Some of the markers described herein are also referred to as hybridization markers when located on an indel region, such as the non-collinear region described herein. This is because the insertion region is, by definition, a polymorphism vis a vis a plant without the insertion. Thus, the marker need only indicate whether the indel region is present or absent. Any suitable marker detection technology may be used to identify such a hybridization marker, e.g. SNP technology is used in the examples provided herein.

The term “plant” includes whole plants, plant cells, plant protoplast, plant cell or tissue culture from which plants can be regenerated, plant calli, plant clumps and plant cells that are intact in plants or parts of plants, such as seeds, flowers, cotyledons, leaves, stems, buds, roots, root tips and the like. As used herein, a “modified plant” means any plant that has a genetic change due to human intervention. A modified plant may have genetic changes introduced through plant transformation, genome editing (including gene replacement or allele replacement), or conventional plant breeding.

The terms “polynucleotide”, “polynucleotide sequence”, “nucleic acid sequence”, “nucleic acid fragment”, and “isolated nucleic acid fragment” are used interchangeably herein. These terms encompass nucleotide sequences and the like. A polynucleotide may be a polymer of RNA or DNA that is single- or double-stranded, that optionally contains synthetic, non-natural or altered nucleotide bases. A polynucleotide in the form of a polymer of DNA may be comprised of one or more segments of cDNA, genomic DNA, synthetic DNA, or mixtures thereof.

The terms “plasmid”, “vector” and “cassette” refer to an extra chromosomal element often carrying genes that are not part of the central metabolism of the cell, and usually in the form of circular double-stranded DNA fragments. Such elements may be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell.

As used herein “altered”, “alteration” or the like in the context of expression (e.g. of RNA or of a protein), refers to any detectable ‘increase’/‘induction’ or ‘decrease’/‘reduction’ in an experimental group (e.g., spinach plant with a modification described herein) as compared to a control group (e.g., wild-type spinach plant that does not comprise the modification).

As used herein “reduced,” “reduction,” or the like refers to any detectable decrease in an experimental group (e.g., spinach plant with a modification described herein) as compared to a control group (e.g., wild-type spinach plant that does not comprise the modification). Accordingly, reduced expression of a protein comprises any detectable decrease in the total level of the protein in a sample and can be determined using routine methods in the art such as, for example, Western blotting and ELISA.

As used herein “induced,” “induction,” or the like in the context of expression (e.g. of RNA or of a protein) refers to any detectable increase in an experimental group (e.g., spinach plant with a modification described herein) as compared to a control group (e.g., wild-type spinach plant that does not comprise the modification). Accordingly, increased expression of a protein comprises any detectable increase in the total level of the protein in a sample and can be determined using routine methods in the art such as, for example, Western blotting and ELISA.

The term “single nucleotide polymorphism” or “SNP” is a DNA sequence variation occurring when a single nucleotide—A, T, C or G—in the genome (or other shared sequence) differs between members of a species (or between paired chromosomes in an individual). For example, two sequenced DNA fragments from different individuals, AAGCCTA to AAGCTTA, contain a difference in a single nucleotide. In this case we say that there are two alleles: C and T. Almost all common SNPs have only two alleles.

“Spinach” or “cultivated spinach” or “cultivated Spinacia oleracea” refers herein to plants of the species Spinacia oleracea (or seeds from which the plants can be grown), and parts of such plants, bred by humans for food and having good agronomic characteristics. This includes any cultivated spinach, such as breeding lines (e.g. backcross lines, inbred lines), cultivars and varieties (open pollinated or hybrids). This includes any type of spinach, such as savoy, flat- or smooth-leaf spinach or semi-savoy types.

“Targeted DNA modification” can be used synonymously with targeted DNA mutation and refers to the introduction of a site specification modification that alters or changes the nucleotide sequence at a specific genomic locus of the plant (e.g., spinach). The targeted DNA modification described herein may be any modification known in the art such as, for example, insertion, deletion, single nucleotide polymorphism (SNP), and or a polynucleotide modification. Additionally, the targeted DNA modification in the genomic locus may be located anywhere in the genomic locus, such as, for example, a coding region of the encoded polypeptide (e.g., exon), a non-coding region (e.g., intron), a regulatory element, or untranslated region. The type and location of the targeted DNA modification is not particularly limited so long as the targeted DNA modification results in altered expression or activity of the protein encoded by the S-gene. In certain embodiments the targeted DNA modification is a deletion of one or more nucleotides, preferably contiguous, of the genomic locus.

An “unfavorable allele” of a marker is a marker allele that segregates with the unfavorable plant phenotype, therefore providing the benefit of identifying plants that can be removed from a breeding program or planting.

Marker loci that demonstrate statistically significant co-segregation with a disease resistance trait that confers broad resistance against a specified disease or diseases are provided herein. Detection of these loci or additional linked loci and the resistance gene may be used in marker assisted selection as part of a breeding program to produce plants that have resistance to a disease or diseases.

### Genetic Mapping

It has been recognized for quite some time that specific genetic loci correlating with particular phenotypes, such as disease resistance, can be mapped in an organism's genome. The plant breeder can advantageously use molecular markers to identify desired individuals by detecting marker alleles that show a statistically significant probability of co-segregation with a desired phenotype, manifested as linkage disequilibrium. By identifying a molecular marker or clusters of molecular markers that co-segregate with a trait of interest, the breeder is able to rapidly select a desired phenotype by selecting for the proper molecular marker allele (a process called marker-assisted selection, or MAS). A variety of methods are available for detecting molecular markers or clusters of molecular markers that co-segregate with a trait of interest, such as a disease resistance trait. The basic idea underlying these methods is the detection of markers, for which alternative genotypes (or alleles) have significantly different average phenotypes. Thus, one makes a comparison among marker loci of the magnitude of difference among alternative genotypes (or alleles) or the level of significance of that difference. Trait genes are inferred to be located nearest the marker(s) that have the greatest associated genotypic difference. Two such methods used to detect trait loci of interest are: 1) Population-based association analysis (i.e. association mapping) and 2) Traditional linkage analysis.

### Association Mapping

Understanding the extent and patterns of linkage disequilibrium (LD) in the genome is a prerequisite for developing efficient association approaches to identify and map quantitative trait loci (QTL). Linkage disequilibrium (LD) refers to the non-random association of alleles in a collection of individuals. When LD is observed among alleles at linked loci, it is measured as LD decay across a specific region of a chromosome. The extent of the LD is a reflection of the recombinational history of that region. The average rate of LD decay in a genome can help predict the number and density of markers that are required to undertake a genome-wide association study and provides an estimate of the resolution that can be expected.

Association or LD mapping aims to identify significant genotype-phenotype associations. It has been exploited as a powerful tool for fine mapping in outcrossing species such as humans (Corder et al. (1994) “Protective effect of apolipoprotein-E type-2 allele for late-onset Alzheimer-disease,” Nat Genet 7:180-184; Hastbacka et al. (1992) “Linkage disequilibrium mapping in isolated founder populations: diastrophic dysplasia in Finland,” Nat Genet 2:204-211; Kerem et al. (1989) “Identification of the cystic fibrosis gene: genetic analysis,” Science 245:1073-1080) and maize (Remington et al., (2001) “Structure of linkage disequilibrium and phenotype associations in the maize genome,” Proc Natl Acad Sci USA 98:11479-11484; Thornsberry et al. (2001) “Dwarf8 polymorphisms associate with variation in flowering time,” Nat Genet 28:286-289; reviewed by Flint-Garcia et al. (2003) “Structure of linkage disequilibrium in plants,” Annu Rev Plant Biol. 54:357-374), where recombination among heterozygotes is frequent and results in a rapid decay of LD. In inbreeding species where recombination among homozygous genotypes is not genetically detectable, the extent of LD is greater (i.e., larger blocks of linked markers are inherited together) and this dramatically enhances the detection power of association mapping (Wall and Pritchard (2003) “Haplotype blocks and linkage disequilibrium in the human genome,” Nat Rev Genet 4:587-597).

The recombinational and mutational history of a population is a function of the mating habit as well as the effective size and age of a population. Large population sizes offer enhanced possibilities for detecting recombination, while older populations are generally associated with higher levels of polymorphism, both of which contribute to observably accelerated rates of LD decay. On the other hand, smaller effective population sizes, e.g., those that have experienced a recent genetic bottleneck, tend to show a slower rate of LD decay, resulting in more extensive haplotype conservation (Flint-Garcia et al. (2003) “Structure of linkage disequilibrium in plants,” Annu Rev Plant Biol. 54:357-374).

Association analyses use quantitative phenotypic scores (e.g., disease tolerance rated from one to nine for each line) in the analysis (as opposed to looking only at tolerant versus resistant allele frequency distributions in intergroup allele distribution types of analysis). The availability of detailed phenotypic performance data collected by breeding programs over multiple years and environments for a large number of elite lines provides a valuable dataset for genetic marker association mapping analyses. This paves the way for a seamless integration between research and application and takes advantage of historically accumulated data sets. However, an understanding of the relationship between polymorphism and recombination is useful in developing appropriate strategies for efficiently extracting maximum information from these resources.

This type of association analysis neither generates nor requires any map data, but rather is independent of map position. This analysis compares the plants' phenotypic score with the genotypes at the various loci. Subsequently, any suitable map (for example, a composite map) can optionally be used to help observe distribution of the identified QTL markers and/or QTL marker clustering using previously determined map locations of the markers.

### Traditional Linkage Analysis

The same principles underlie traditional linkage analysis; however, LD is generated by creating a population from a small number of founders. The founders are selected to maximize the level of polymorphism within the constructed population, and polymorphic sites are assessed for their level of cosegregation with a given phenotype. A number of statistical methods have been used to identify significant marker-trait associations. One such method is an interval mapping approach (Lander and Botstein, Genetics 121:185-199 (1989), in which each of many positions along a genetic map (say at 1 cM intervals) is tested for the likelihood that a gene controlling a trait of interest is located at that position. The genotype/phenotype data are used to calculate for each test position a LOD score (log of likelihood ratio). When the LOD score exceeds a threshold value, there is significant evidence for the location of a gene controlling the trait of interest at that position on the genetic map (which will fall between two particular marker loci).

Marker loci that demonstrate statistically significant co-segregation with a disease resistance trait, as determined by traditional linkage analysis and by Genome Wide Association Studies (GWAS), are provided herein. Detection of these loci or additional linked loci can be used in marker assisted breeding programs to produce plants having disease resistance.

Activities in marker assisted breeding programs may include but are not limited to: selecting among new breeding populations to identify which population has the highest frequency of favorable nucleic acid sequences based on historical genotype and agronomic trait associations, selecting favorable nucleic acid sequences among progeny in breeding populations, selecting among parental lines based on prediction of progeny performance, and advancing lines in germplasm improvement activities based on presence of favorable nucleic acid sequences.

### Markers

The present invention provides molecular markers, (i.e., including marker loci and nucleic acids corresponding to (or derived from) these marker loci, such as probes and amplification products) useful for genotyping plants, correlated with a susceptibility(S)) gene in spinach, for example Spov3_chr4_93275081, Spov3_chr4_105049870, Spov3_chr4_107241402, Spov3_chr4_109546568, Spov3_chr4_109698808, Spov3_chr4_112008390, Spov3_chr4_112574123, Spov3_chr4_117783935, Spov3_chr4_118191085, Spov3_chr4_118788268, and Spov3_chr4_121142541 described below. Such molecular markers are useful for selecting spinach plants that are resistant to downy mildew. Accordingly, these markers are useful for marker assisted selection (MAS) and breeding of downy mildew resistant lines and identification of resistant lines. The markers of the invention are also used to identify and define chromosome intervals corresponding to an S-gene. An S-gene can be isolated by positional cloning, e.g. of the genetic interval defined by a pair of markers described herein or subsequences of an interval defined by and including such markers. In addition, an S-gene isolated from one organism, e.g. spinach, can, in turn, serve to isolate homologues of an S-gene in other organisms, including a variety of commercially important crops.

As is known to those skilled in the art, there are many kinds of molecular markers. For example, molecular markers can include restriction fragment length polymorphisms (RFLP), random amplified polymorphic DNA (RAPD), amplified fragment length polymorphisms (AFLP), single nucleotide polymorphisms (SNP) or simple sequence repeats (SSR).

Simple sequence repeats (SSR) or microsatellites are regions of DNA where one to a few bases are tandemly repeated for few to hundreds of times. For example, a di-nucleotide repeat would resemble CACACACA and a trinucleotide repeat would resemble ATGATGATGATG. Simple sequence repeats are thought to be generated due to slippage mediated errors during DNA replication, repair and recombination. Over time, these repeated sequences vary in length between one cultivar and another. An example of allelic variation in SSRs would be: Allele A being GAGAGAGA (4 repeats of the GA sequence) and allele B being GAGAGAGAGA (6 repeats of the GA sequence). When SSRs occur in a coding region, their survival depends on their impact on structure and function of the encoded protein. Since repeat tracks are prone to DNA-slippage mediated expansions/deletions, their occurrences in coding regions are limited by non-perturbation of the reading frame and tolerance of expanding amino acid stretches in the encoded proteins. Among all possible SSRs, tri-nucleotide repeats or multiples thereof are more common in coding regions.

A single nucleotide polymorphism (SNP) is a DNA sequence variation occurring when a single nucleotide—A, T, C or G—differs between members of a species (or between paired chromosomes in an individual). For example, two sequenced DNA fragments from two individuals, AAGCCTA to AAGCTTA, contain a difference in a single nucleotide. In this case, there are two alleles: C and T.

TABLE 1

| | | Position | Reference | Alternate |
|---|---|---|---|---|
| Marker | Chromosome | (bp) | Allele | Allele |
| Spov3_chr4_93275081 | 4 | 93,275,081 | C | T |
| Spov3_chr4_105049870 | 4 | 105,049,870 | G | A |
| Spov3_chr4_107241402 | 4 | 107,241,402 | G | A |
| Spov3_chr4_109546568 | 4 | 109,546,568 | G | A |
| Spov3_chr4_109698808 | 4 | 109,698,808 | G | A |
| Spov3_chr4_112008390 | 4 | 112,008,390 | G | A |
| Spov3_chr4_112574123 | 4 | 112,574,123 | C | T |
| Spov3_chr4_117783935 | 4 | 117,783,935 | C | T |
| Spov3_chr4_118191085 | 4 | 118,191,085 | G | A |
| Spov3_chr4_118788268 | 4 | 118,788,268 | G | A |
| Spov3_chr4_121142541 | 4 | 121,142,541 | G | A |

>Spov3_chr4_93275081-SEQ ID NO: 241 ('C' or 'T' at position 101)
TTTCATGGTGGTGTGGTACAGGTTGAAAAGTTTGTTCAAGATGCCATCTCAAAGGTAACTA
ATTCGTTTTTGGTACACACTAGCCTACTTCCCCTCCTCTYTTGGTTCTTTATGCATAAGCTC
TGAAATCCTTTAACATGAATAGGAGAGCATTGCAATGTTGATGAATTCTGAAGTTCATCCC
CCGCCCCCTTGGTACTC
>Spov3_chr4_105049870-SEQ ID NO: 242 ('G' or 'A' at position 101)
AGAACCAATTAGTTAAACATTGGAACCAATTAGTTAAGCAATGTAACTAATTAGTCAAACA
ATGGGACCAAATAGTTAAGCGATGAAACCAATTAGTTAARCAATCAAATCGGTCTTTTCTG
TAATGCGGTCTTACACAAAAGTTGTCGTTTGAAGTTAGAGTCGATTTCTTTTCACCACTGT
TTTTGGGAGGCAAATCAC
>Spov3_chr4_107241402-SEQ ID NO: 243 ('G' or 'A' at position 101)
TTTTAAGTGTTTATATATAGTGGTGGATATTAGTTGTGCTTAGGGTGGGCCCGACCCCTCT
AGGCGAAAAATTATGTACGGAGTAAAGTACTTTTTTTTARTTATGATCCCCTTAAACTTTGT
GTATAATTGTAAATTACACGGAGTAATATATTACTTCATTTTCTACTGTCGTTCTCATAAAG
CTATAAGGTCCCACGA
>Spov3_chr4_109546568-SEQ ID NO: 244 ('G' or 'A' at position 101)
ATTCACGAAATCTTGGAGATAACGAAAGAATTCCTCGCCGAATTATGATTGCAGGTTACTA
TGATATACCAAAGTGATGAGTTGAGAGCCACAAATATTGRAACTATGAAGATATATTGAAT
GCAACACTAATATCTGATAACTTATATCATTAGCTATTTGCTTTAGCTGATAGTCATTGATT
GTTGGGAGTTTCTTGAC
>Spov3_chr4_109698808-SEQ ID NO: 245 ('G' or 'A' at position 101)
GTTTACGGATTAGTGAGATAAAAGAGTTTCAAGAGTTGATTTCTGGGCTGATTTTGCATTT
TGGAGGATATTTTTACAATTTTGRGTGTTTGGTGTTTTTRTGGAGTAGGTGAGCTGGAGA
ATAATGCGTTTTGGAGTTACATTATGATGATGATGATGGCTTACACAAATTTTGGGGTAGA
AAAGCGATGATGGTTAGCA
>Spov3_chr4_112008390-SEQ ID NO: 246 ('G' or 'A' at position 101)
TAATTTTTACAAGGGTAGTGATGTACTTTCACCATTAAAATGAGGGTGCTTTTAGCGGAAT
AGGACGTCGGATAAAATCATACTCGTGATATTAGGGCTTRAAATACATAGTAGTTGAATTA
AGCTAGTGYTCTAACCGCCTCTCCCTCCCTTCTTACGAAATACTGCCTCTGTTCCACAATA
CATTCATCATTTCTTTTT
>Spov3_chr4_112574123-SEQ ID NO: 247 ('C' or 'T' at position 101)
GCCATTTATAGAAACGGTGCAAGTAATCCGGGACGGCTTTATAAGGAAAGCGGTGCAAGT
ATTCCGGAACGGAGGGAATAATATTCAAGAAATATAAATTYTGATTTAGATAAACCTCTAC
TACATATTTCGTAGTAGGGTAAGAGAAGTTTGTCAACTTTAACATTTGACAAYATGATAAG
CAAAAGAAGACTACGAACG
>Spov3_chr4_117783935-SEQ ID NO: 248 ('C' or 'T' at position 101)
AAGACCATTCAACATAACCCCATCAAATTTAACTCTAGGAAAACACCTTATAATCTCATCCC
ACATACCCAATTTACAAAATCCTCGAAAAACCAAATTCYAAACAGCAACATTCCTCTCTGG
CATTTCATTCAAAACTTTCAACCCATCATCAACAAACCCCATTTGCACATAAAGATCAACGA
TAGAACTCCCAATGTA
>Spov3_chr4_118191085-SEQ ID NO: 249 ('G' or 'A' at position 101)
TGTAAGGTCTCAGCATTGGTGCATATTAGAGAGCTATCATCTACAGGTTGGTTTTGAGGT
GAGATCTGAGCAACCATACAACAGAAGAGTATCACTCTCCRGATGTTCTTTGACTGGTCA
AGAGGGAGACTACGTTGTGCTTCCAAGTAACATCCAGGAGTTGTATGTTGATAATTGCAG
AGACTTTCGTTGCCTATCTGA
>Spov3_chr4_118788268-SEQ ID NO: 250 ('G' or 'A' at position 101)
CGGATGGAGTAGTAAATACAAAAAAAATTGATACAAAAGTGGTGTATTTTTAAGGTAATAA
ATACCAAAAAATCCTACAAAATATCCAATTAACAAATACRAGTACGTAGTACAAGTAGATC
ATACAAACATACTATGATTTTCCCGTATCTATATCGTATCGAAACTTGTGTTTGTGTTCATC
ATCGTCCAATAAAGGGC
>Spov3_chr4_121142541-SEQ ID NO: 251 ('G' or 'A' at position 101)
TGTAATGTAATAATGCAAATATAATGTACTCGAGTAAGTGATAGTCACAGGTTCGATTCCT
CTATCCTCCACTTGTAATTTGCACGGACTACAGAATATARTCCATGTGGCTCATTCGCACC
AAGAAAATGCCTACACAGAAATATTAAGATAAATAGAAAAACTTACAAAAAAGAGTTGCAG
AACAAGGACACTGATCTG

### Linked Markers

Those of skill in the art will recognize that additional molecular markers can be identified within the intervals defined by the above described pair of markers (e.g., Spov3_chr4_93275081 and Spov3_chr4_121142541). Such markers are also genetically linked to an S-gene, and are within the scope of the present invention. Markers can be identified by any of a variety of genetic or physical mapping techniques. Methods of determining whether markers are genetically linked to an S-gene are known to those of skill in the art and include, for example, interval mapping (Lander and Botstein, (1989) Genetics 121:185), regression mapping (Haley and Knott, (1992) Heredity 69:315) MQM or mapping (Jansen, (1994) Genetics 138:871). In addition, such physical mapping techniques as chromosome walking, contig mapping and assembly, and the like, can be employed to identify and isolate additional sequences useful as markers in the context of the present invention.

### Chromosomal Intervals

Chromosomal intervals that correlate with the disease resistance trait are provided. A variety of methods are available for identifying chromosomal intervals. The boundaries of such chromosomal intervals are drawn to encompass markers that will be linked to the gene(s) controlling the trait of interest. In other words, the chromosomal interval is drawn such that any marker that lies within that interval (including the terminal markers that define the boundaries of the interval) can be used as a marker for a disease resistance trait.

Conversely, e.g., if two markers in close proximity show co-segregation with the desired phenotypic trait, it is sometimes unclear if each of those markers identify the same gene or two different gene or multiple genes. Regardless, knowledge of how many genes are in a particular physical/genomic interval is not necessary to make or practice that which is presented in the current disclosure.

The chromosome 4 interval may encompass any of the markers identified herein as being associated with the DM resistance trait including a “T’ at Spov3_chr4_93275081 (position 101 of reference sequence SEQ ID NO: 241), an “A” at Spov3_chr4_105049870 (position 101 of reference sequence SEQ ID NO: 242), an “A” at Spov3_chr4_107241402 (position 101 of reference sequence SEQ ID NO: 243), an “A” at Spov3_chr4_109546568 (position 101 of reference sequence SEQ ID NO: 244), an “A” at Spov3_chr4_109698808 (position 101 of reference sequence SEQ ID NO: 245), an “A” at Spov3_chr4_112008390 (position 101 of reference sequence SEQ ID NO: 246), a “T” at Spov3_chr4_112574123 (position 101 of reference sequence SEQ ID NO: 247), a “T” at Spov3_chr4_117783935 (position 101 of reference sequence SEQ ID NO: 248), an “A” at Spov3_chr4_118191085 (position 101 of reference sequence SEQ ID NO: 249), an “A” at Spov3_chr4_118788268 (position 101 of reference sequence SEQ ID NO: 250), and an “A” at Spov3_chr4_121142541 (position 101 of reference sequence SEQ ID NO: 251). Any marker located within these intervals can find use as a marker for DM resistance and can be used in the context of the methods presented herein to identify and/or select plants that have resistance to DM, whether it is newly conferred or enhanced compared to a control plant.

Chromosomal intervals can also be defined by markers that are linked to (show linkage disequilibrium with) a disease resistant gene, and r² is a common measure of linkage disequilibrium (LD) in the context of association studies. If the r² value of LD between a chromosome 4 marker locus in an interval of interest and another chromosome 4 marker locus in close proximity is greater than ⅓ (Ardlie et al, Nature Reviews Genetics 3:299-309 (2002)), the loci are in linkage disequilibrium with one another.

### Detection of Marker Loci

Markers corresponding to genetic polymorphisms between members of a population can be detected by numerous methods, well-established in the art (e.g., restriction fragment length polymorphisms, isozyme markers, allele specific hybridization (ASH), amplified variable sequences of the plant genome, self-sustained sequence replication, simple sequence repeat (SSR), single nucleotide polymorphism (SNP) or amplified fragment length polymorphisms (AFLP)).

The majority of genetic markers rely on one or more property of nucleic acids for their detection. For example, some techniques for detecting genetic markers utilize hybridization of a probe nucleic acid to nucleic acids corresponding to the genetic marker. Hybridization formats include but are not limited to, solution phase, solid phase, mixed phase or in situ hybridization assays. Markers which are restriction fragment length polymorphisms (RFLP), are detected by hybridizing a probe (which is typically a sub-fragment or a synthetic oligonucleotide corresponding to a sub-fragment of the nucleic acid to be detected) to restriction digested genomic DNA. The restriction enzyme is selected to provide restriction fragments of at least two alternative (or polymorphic) lengths in different individuals and will often vary from line to line. Determining a (one or more) restriction enzyme that produces informative fragments for each cross is a simple procedure, well known in the art. After separation by length in an appropriate matrix (e.g., agarose) and transfer to a membrane (e.g., nitrocellulose, nylon), the labeled probe is hybridized under conditions which result in equilibrium binding of the probe to the target followed by removal of excess probe by washing.

Nucleic acid probes to the marker loci can be cloned and/or synthesized. Detectable labels suitable for use with nucleic acid probes include any composition detectable by radio-isotopic, spectroscopic, photochemical, biochemical, immunochemical, electrical, optical or chemical means. Useful labels include biotin for staining with labeled streptavidin conjugate, magnetic beads, fluorescent dyes, radiolabels, enzymes and colorimetric labels. Other labels include ligands which bind to antibodies labeled with fluorophores, chemiluminescent agents and enzymes. Labeling markers is readily achieved such as by the use of labeled PCR primers to marker loci.

The hybridized probe is then detected using, most typically, autoradiography or other similar detection technique (e.g., fluorography, liquid scintillation counter, etc.). Examples of specific hybridization protocols are widely available in the art, see, e.g., Berger, Sambrook, Ausubel, all supra.

Amplified variable sequences refer to amplified sequences of the plant genome which exhibit high nucleic acid residue variability between members of the same species. All organisms have variable genomic sequences and each organism (with the exception of a clone) has a different set of variable sequences. Once identified, the presence of specific variable sequence can be used to predict phenotypic traits. Preferably, DNA from the plant serves as a template for amplification with primers that flank a variable sequence of DNA. The variable sequence is amplified and then sequenced.

In vitro amplification techniques are well known in the art. Examples of techniques sufficient to direct persons of skill through such in vitro methods, including the polymerase chain reaction (PCR), the ligase chain reaction (LCR), QB-replicase amplification and other RNA polymerase mediated techniques (e.g., NASBA), are found in Berger, Sambrook and Ausubel (all supra) as well as Mullis, et al., (1987) U.S. Pat. No. 4,683,202; PCR Protocols, A Guide to Methods and Applications (Innis, et al., eds.) Academic Press Inc., San Diego Academic Press Inc. San Diego, Calif. (1990) (Innis); Arnheim and Levinson, (Oct. 1, 1990) C&EN 36-47; The Journal Of NIH Research (1991) 3:81-94; (Kwoh, et al., (1989) Proc. Natl. Acad. Sci. USA 86:1173; Guatelli, et al., (1990) Proc. Natl. Acad. Sci. USA 87:1874; Lomeli, et al., (1989) J. Clin. Chem 35:1826; Landegren, et al. (1988) Science 241:1077-1080; Van Brunt, (1990) Biotechnology 8:291-294; Wu and Wallace, (1989) Gene 4:560; Barringer, at al., (1990) Gene 89:117 and Sooknanan and Malek, (1995) Biotechnology 13:563-564. Improved methods of cloning in vitro amplified nucleic acids are described in Wallace, et al., U.S. Pat. No. 5,426,039. Improved methods of amplifying large nucleic acids by PCR are summarized in Cheng, et al., (1994) Nature 369:684, and the references therein, in which PCR amplicons of up to 40 kb are generated. One of skill will appreciate that essentially any RNA can be converted into a double stranded DNA suitable for restriction digestion, PCR expansion and sequencing using reverse transcriptase and a polymerase. See, Ausubel, Sambrook and Berger, all supra.

Oligonucleotides for use as primers, e.g., in amplification reactions and for use as nucleic acid sequence probes, are typically synthesized chemically according to the solid phase phosphoramidite triester method described by Beaucage and Caruthers, (1981) Tetrahedron Lett. 22:1859 or can simply be ordered commercially.

Alternatively, self-sustained sequence replication can be used to identify genetic markers. Self-sustained sequence replication refers to a method of nucleic acid amplification using target nucleic acid sequences which are replicated exponentially in vitro under substantially isothermal conditions by using three enzymatic activities involved in retroviral replication: (1) reverse transcriptase, (2) Rnase H and (3) a DNA-dependent RNA polymerase (Guatelli, et al., (1990) Proc Natl Acad Sci USA 87:1874). By mimicking the retroviral strategy of RNA replication by means of cDNA intermediates, this reaction accumulates cDNA and RNA copies of the original target

As mentioned above, there are many different types of molecular markers, including amplified fragment length polymorphisms (AFLP), allele-specific hybridization (ASH), single nucleotide polymorphisms (SNP), simple sequence repeats (SSR) and isozyme markers. Methods of using the different types of molecular markers are known to those skilled in the art. The markers of the present invention include simple sequence repeats and single nucleotide polymorphisms.

### Kits

Kits are also provided to facilitate the screening of germplasm for the markers of the present invention. The kits comprise the polynucleotides of the present invention, fragments or complements thereof, for use as probes or primers to detect the markers for an S-gene. Instructions for using the polynucleotides, as well as buffers and/or other solutions may also be provided to facilitate the use of the polynucleotides.

### Marker Assisted Selection and Breeding of Plants

The spinach plants of the disclosure may be used in a plant breeding program. The goal of plant breeding is to combine, in a single variety or hybrid, various desirable agronomic traits. For field crops, these traits may include, for example, resistance to diseases and insects, tolerance to heat and drought, tolerance to chilling or freezing, reduced time to crop maturity, greater yield and better agronomic quality. With mechanical harvesting of many crops, uniformity of plant characteristics such as germination and stand establishment, growth rate, maturity and plant and ear height is desirable. Traditional plant breeding is an important tool in developing new and improved commercial crops. This disclosure encompasses methods for producing a plant by crossing a first parent plant with a second parent plant wherein one or both of the parent plants is a plant displaying a phenotype as described herein.

Plant breeding techniques known in the art and used in a plant breeding program include, but are not limited to, recurrent selection, bulk selection, mass selection, backcrossing, pedigree breeding, open pollination breeding, restriction fragment length polymorphism enhanced selection, genetic marker enhanced selection, doubled haploids and transformation. Often combinations of these techniques are used. Marker loci and the alleles provided herein can be used in marker assisted selection (MAS) breeding. The more tightly linked a marker is with a DNA locus influencing a phenotype (e.g., disease resistance), the more reliable the marker is in MAS, as the likelihood of a recombination event unlinking the marker and the locus decreases. Markers containing the causal polymorphism for a trait, or that are within the coding sequence of a causative gene, are ideal as no recombination is expected between them and the sequence of DNA responsible for the phenotype. However, markers do not need to contain or correspond to casual polymorphisms in order to be effective in MAS. In fact, most MAS breeding only uses markers linked to a causal mutation.

Developing molecular markers in crop species can increase efficiency in plant breeding through MAS. Genetic markers are used to identify plants that contain a desired genotype at one or more loci, and that are expected to transfer the desired genotype, along with a desired phenotype to their progeny. Genetic markers can be used to identify plants containing a desired genotype at one locus, or at several unlinked or linked loci (e.g., a haplotype), and that would be expected to transfer the desired genotype, along with a desired phenotype to their progeny.

In general, MAS uses polymorphic markers that have been identified as having a significant likelihood of co-segregation with a desired trait. Such markers are presumed to map near a gene or genes that give the plant its desired phenotype, and are considered indicators for the desired trait.

Identification of plants or germplasm that include a marker locus or marker loci linked to a desired trait or traits provides a basis for performing MAS. Plants that comprise favorable markers or favorable alleles are selected for, while plants that comprise markers or alleles that are negatively correlated with the desired trait can be selected against. Desired markers and/or alleles can be introgressed into plants having a desired (e.g., elite or exotic) genetic background to produce an introgressed plant or germplasm having the desired trait. In some aspects, it is contemplated that a plurality of markers for desired traits are sequentially or simultaneous selected and/or introgressed. The combinations of markers that are selected for in a single plant is not limited, and can include any combination of markers disclosed herein or any marker linked to the markers disclosed herein.

The present disclosure provides the means to identify disease resistant spinach plants that comprise altered expression of an S-gene by identifying plants having a specified allele, e.g., at one or more of markers Spov3_chr4_93275081, Spov3_chr4_105049870, Spov3_chr4_107241402, Spov3_chr4_109546568, Spov3_chr4_109698808, Spov3_chr4_112008390, Spov3_chr4_112574123, Spov3_chr4_117783935, Spov3_chr4_118191085, Spov3_chr4_118788268, and Spov3_chr4_121142541. Similarly, by identifying plants lacking the desired allele, plants without the disease resistance can be identified and, e.g., eliminated from subsequent crosses.

MAS is a powerful shortcut to select for desired phenotypes and for introgressing desired traits into cultivars (e.g., introgressing desired traits into elite lines). MAS is easily adapted to high throughput molecular analysis methods that can quickly screen large numbers of plant or germplasm genetic material for the markers of interest and is much more cost effective than cultivating and observing plants for visible traits.

When a population is segregating for multiple loci affecting one of multiple traits, e.g., multiple loci involved in disease resistance, the efficiency of MAS compared to phenotypic screening becomes even greater, because all of the loci can be evaluated together from a single sample of DNA.

Another use of MAS in plant breeding is to assist the recovery of the recurrent parent genotype by backcross breeding. Backcross breeding is the process of crossing a progeny back to one of its parents. Backcrossing is usually done for the purpose of introgressing one or a few loci from a donor parent into an otherwise desirable genetic background from the recurrent parent. The more cycles of backcrossing that are done, the greater the genetic contribution of the recurrent parent to the resulting variety. This is often necessary, because donor parent plants may be otherwise undesirable, i.e., due to low yield, low fecundity or the like. In contrast, varieties which are the result of intensive breeding programs may have excellent yield, fecundity or the like, merely being deficient in one desired trait. As a skilled worker understands, backcrossing can be done to select for or against a trait.

The introgression of one or more desired loci from a donor line into another is achieved via repeated backcrossing to a recurrent parent accompanied by selection to retain one or more loci from the donor parent. Markers associated with sex determination are assayed in progeny and those progeny with one or more desired markers are selected for advancement. In another aspect, one or more markers can be assayed in the progeny to select for plants with the genotype of the parent. This invention anticipates that trait introgression activities will require more than one generation, wherein progeny are crossed to the recurrent parent or selfed. Selections are made based on the presence of one or more sex determination markers and can also be made based on the recurrent parent genotype, wherein screening is performed on a genetic marker and/or phenotype basis. In another embodiment, the markers can be used in conjunction with other markers, ideally at least one on each chromosome of the spinach genome, to track the introgression into elite germplasm.

### Positional Cloning

The molecular markers of the present invention, for example, Spov3_chr4_93275081, Spov3_chr4_105049870, Spov3_chr4_107241402, Spov3_chr4_109546568, Spov3_chr4_109698808, Spov3_chr4_112008390, Spov3_chr4_112574123, Spov3_chr4_117783935, Spov3_chr4_118191085, Spov3_chr4_118788268, Spov3_chr4_121142541, etc., and nucleic acids homologous thereto, can be used, as indicated previously, to identify additional linked marker loci, which can be cloned by well established procedures, e.g., as described in detail in Ausubel, Berger and Sambrook, supra. Similarly, these markers and genes as well as any additionally identified linked molecular markers can be used to physically isolate, e.g., by cloning, nucleic acids associated with markers contributing to fertility restoration. Such nucleic acids, i.e., linked to the marker, have a variety of uses, including as genetic markers for identification of additional markers in subsequent applications of marker assisted selection (MAS). Such nucleic acids may also include an S-gene itself.

These nucleic acids are first identified by their genetic linkage to markers of the present invention. Isolation of the nucleic acid of interest is achieved by any number of methods as discussed in detail in such references as Ausubel, Berger and Sambrook, supra, and Clark, Ed. (1997) Plant Molecular Biology: A Laboratory Manual Springer-Verlag, Berlin.

For example, “Positional gene cloning” uses the proximity of a genetic marker to physically define an isolated chromosomal fragment that is linked to a gene. The isolated chromosomal fragment can be produced by such well known methods as digesting chromosomal DNA with one or more restriction enzymes or by amplifying a chromosomal region in a polymerase chain reaction (PCR) or alternative amplification reaction or by direct Sanger sequencing. The digested or amplified fragment is typically ligated into a vector suitable for replication, e.g., a plasmid, a cosmid, a phage, an artificial chromosome, or the like and optionally expression, of the inserted fragment. Markers which are adjacent to an open reading frame (ORF) associated with a phenotypic trait can hybridize to a DNA clone, thereby identifying a clone on which an ORF is located. If the marker is more distant, a fragment containing the open reading frame is identified by successive rounds of screening and isolation of clones which together comprise a contiguous sequence of DNA, a “contig.” Protocols sufficient to guide one of skill through the isolation of clones associated with linked markers are found in, e.g. Berger, Sambrook and Ausubel, all supra.

### Isolated Chromosome Region and Isolated S-Gene

The present invention provides the chromosome region comprising sequences associated with an S-gene involved in disease resistance. The gene is localized in the region defined by two markers of the present invention (Spov3_chr4_93275081 (SEQ ID NO: 241) and Spov3_chr4_121142541 (SEQ ID NO: 251)) wherein each marker is genetically linked to the gene. A chromosome region can contain one or more ORFs associated with disease resistance, and can be cloned on one or more individual vectors, e.g., depending on the size of the chromosome region. For example, in the present invention three genes were identified within the interval flanked by SNP markers Spov3_chr4_93275081 and Spov3_chr4_121142541. In general, there are three recognized groups of S-genes acting during different stages of infection: early pathogen establishment, modulation of host defenses, and pathogen sustenance (Annu Rev Phytopathol. 2014; 52:551-81).

The present invention provides, inter alia, isolated nucleic acids of DNA, RNA, homologs, paralogs and orthologs and/or chimeras thereof, comprising an S-gene polynucleotide and proteins encoded thereby. This includes naturally occurring as well as synthetic variants and homologs of the sequences.

Clathrin adaptor medium subunit polypeptides are encompassed by the disclosure. “Clathrin adaptor medium subunit polypeptide” and “clathrin adaptor medium subunit protein” as used herein interchangeably refers to a polypeptide(s) associated with disease resistance, and is sufficiently identical to the spinach clathrin adaptor medium subunit polypeptide of SEQ ID NO: 6. A variety of clathrin adaptor medium subunit polypeptides are contemplated.

Pentatricopeptide repeat-containing polypeptides are encompassed by the disclosure. “Pentatricopeptide repeat-containing polypeptide” and “pentatricopeptide repeat-containing protein” as used herein interchangeably refers to a polypeptide(s) associated with disease resistance, and is sufficiently identical to the spinach pentatricopeptide repeat-containing polypeptide of SEQ ID NO: 98. A variety of pentatricopeptide repeat-containing polypeptides are contemplated.

Resistant to Pseudomonas syringae 2-like (RPS2-like) resistance polypeptides are encompassed by the disclosure. “RPS2-like resistance polypeptide” and “RPS2-like resistance protein” as used herein interchangeably refers to a polypeptide(s) associated with disease resistance, and is sufficiently identical to the spinach RPS2-like resistance polypeptide of SEQ ID NO: 174. A variety of RPS2-like resistance polypeptides are contemplated.

“Sufficiently identical” is used herein to refer to an amino acid sequence that has at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity. In some embodiments the sequence identity is against the full length sequence of a polypeptide. The term “about” when used herein in context with percent sequence identity means+/−1.0%.

“Fragments” or “biologically active portions” include polypeptide or polynucleotide fragments comprising sequences sufficiently identical to a clathrin adaptor medium subunit, a pentatricopeptide repeat-containing, or an RPS2-like resistance polypeptide or polynucleotide, respectively.

“Variants” as used herein refers to proteins or polypeptides having an amino acid sequence that is at least about 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or greater identical to the parental amino acid sequence.

In some embodiments a clathrin adaptor medium subunit polypeptide, a pentatricopeptide repeat-containing polypeptide, or an RPS2-like resistance polypeptide comprises an amino acid sequence having at least about 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater identity to the full length or a fragment of the amino acid sequence of SEQ ID NO: 6, SEQ ID NO: 98, or SEQ ID NO: 174.

Sequences homologous, i.e., that share significant sequence identity or similarity, to those provided herein are also an aspect of the invention. Homologous sequences can be derived from any plant including monocots and dicots and in particular agriculturally important plant species, including but not limited to, crops such as soybean, wheat, corn (maize), potato, cotton, rape (including canola), sunflower, alfalfa, clover, sugarcane, and turf; or fruits and vegetables, such as banana, blackberry, blueberry, strawberry, and raspberry, cantaloupe, carrot, cauliflower, coffee, cucumber, eggplant, grapes, honeydew, lettuce, mango, melon, onion, papaya, peas, peppers, pineapple, pumpkin, spinach, squash, sweet corn, tobacco, tomato, tomatillo, watermelon, rosaceous fruits (such as apple, peach, pear, cherry and plum) and vegetable brassicas (such as broccoli, cabbage, cauliflower, Brussels sprouts, and kohlrabi). Other crops, including fruits and vegetables, whose phenotype can be changed and which comprise homologous sequences include basil, barley; rye; millet; sorghum; currant; avocado; hops; citrus fruits such as oranges, lemons, grapefruit and tangerines, artichoke, cherries; endive; leek; roots such as arrowroot, beet, cassava, turnip, radish, yam, and sweet potato; and beans. In addition, homologous sequences may be derived from plants that are evolutionarily-related to crop plants, but which may not have yet been used as crop plants.

Homologous sequences can comprise orthologous or paralogous sequences. Several different methods are known by those of skill in the art for identifying and defining these functionally homologous sequences. Three general methods for defining orthologs and paralogs are described; an ortholog, paralog or homolog may be identified by one or more of the methods described below.

Orthologs and paralogs are evolutionarily related genes that have similar sequence and similar functions. Orthologs are structurally related genes in different species that are derived by a speciation event. Paralogs are structurally related genes within a single species that are derived by a duplication event.

Within a single plant species, gene duplication may result in two copies of a particular gene, giving rise to two or more genes with similar sequence and often similar function known as paralogs. A paralog is therefore a similar gene formed by duplication within the same species. Paralogs typically cluster together or in the same clade (a group of similar genes) when a gene family phylogeny is analyzed using programs such as CLUSTAL (Thompson et al. (1994) Nucleic Acids Res. 22:4673-4680; Higgins et al. (1996) Methods Enzymol. 266:383-402). Groups of similar genes can also be identified with pair-wise BLAST analysis (Feng and Doolittle (1987) J. Mol. Evol. 25:351-360).

For example, a clade of very similar MADS domain transcription factors from Arabidopsis all share a common function in flowering time (Ratcliffe et al. (2001) Plant Physiol. 126:122-132), and a group of very similar AP2 domain transcription factors from Arabidopsis are involved in tolerance of plants to freezing (Gilmour et al. (1998) Plant J. 16:433-442). Analysis of groups of similar genes with similar function that fall within one clade can yield sub-sequences that are particular to the clade. These sub-sequences, known as consensus sequences, can not only be used to define the sequences within each clade, but define the functions of these genes; genes within a clade may contain paralogous sequences, or orthologous sequences that share the same function (see also, for example, Mount (2001), in Bioinformatics: Sequence and Genome Analysis Cold Spring Harbor Laboratory Press, Cold Spring Harbor, N.Y., page 543.)

Speciation, the production of new species from a parental species, can also give rise to two or more genes with similar sequence and similar function. These genes, termed orthologs, often have an identical function within their host plants and are often interchangeable between species without losing function. Because plants have common ancestors, many genes in any plant species will have a corresponding orthologous gene in another plant species. Once a phylogenic tree for a gene family of one species has been constructed using a program such as CLUSTAL (Thompson et al. (1994) Nucleic Acids Res. 22:4673-4680; Higgins et al. (1996) supra) potential orthologous sequences can be placed into the phylogenetic tree and their relationship to genes from the species of interest can be determined. Orthologous sequences can also be identified by a reciprocal BLAST strategy. Once an orthologous sequence has been identified, the function of the ortholog can be deduced from the identified function of the reference sequence.

Orthologous genes from different organisms have highly conserved functions, and very often essentially identical functions (Lee et al. (2002) Genome Res. 12:493-502; Remm et al. (2001) J. Mol. Biol. 314:1041-1052). Paralogous genes, which have diverged through gene duplication, may retain similar functions of the encoded proteins. In such cases, paralogs can be used interchangeably with respect to certain embodiments of the instant invention (for example, transgenic expression of a coding sequence).

Exemplary sequences homologous to the spinach clathrin adaptor medium subunit polynucleotide (SEQ ID NOs: 4 or 5) are set forth in SEQ ID NOS: 7-63, 252-293 and summarized in Tables 2, 5, 6, and 7. SEQ ID NO: 6 relates to the spinach clathrin adaptor medium subunit protein sequence, and homologous protein sequences from other plant species are shown in SEQ ID NOs: 64-92. Additional homologous protein sequences are set forth in SEQ ID NOs: 294-315.

Exemplary sequences homologous to the spinach pentatricopeptide repeat-containing polynucleotide (SEQ ID NOs: 96 or 97) are set forth in SEQ ID NOs: 99-144, 317-325 and summarized in Tables 3, 5, 6, and 7. SEQ ID NO: 98 relates to the pentatricopeptide repeat-containing protein sequence, and homologous protein sequences from other plant species are shown in SEQ ID NOs: 145-168. Additional homologous protein sequences are set forth in SEQ ID NOs: 326-330.

Exemplary sequences homologous to the spinach RPS2-like resistance polynucleotide (SEQ ID NOs: 172 or 173) are set forth in SEQ ID NOs: 175-218, 331-332 and summarized in Tables 4 and 6. Homologous protein sequences are set forth in SEQ ID NOs: 219-240 and 333.

TABLE 2

| Organism | Common name | Gene ID | Locus | Transcript ID | E-value | Bit score | % Identity | % Gaps |
|---|---|---|---|---|---|---|---|---|
| Spinacia | spinach | PAC: 45131357 | Spov3_chr4.04752 | (SEQ ID NO: 5) | | | 100.00 | |
| oleracea | | | (SEQ ID NO: 4) | | | | | |
| Beta vulgaris | beet, swiss | 104897272 | LOC104897272 | XM_010684118.2 | 0 | 874.00 | 97.20 | 0 |
| subsp. | chard | | (SEQ ID NO: 7) | (SEQ ID NO: 35) | | | | |
| vulgaris | | | | | | | | |
| Brassica | rapeseed | 106420212 | LOC106420212 | XM_013861063.2 | 0 | 832.40 | 90.65 | 0 |
| napus | | | (SEQ ID NO: 8) | (SEQ ID NO: 36) | | | | |
| | | 106307948 | LOC106307948 | XM_013745046.1 | 0 | 832.02 | 90.42 | 0 |
| Brassica | wild cabbage | 106307948 | LOC106307948 | XM_013745046.1 | 0 | 832.02 | 90.42 | 0 |
| oleracea var. | | | (SEQ ID NO: 9) | (SEQ ID NO: 37) | | | | |
| oleracea | | | | | | | | |
| Capsicum | pepper | 107855741 | LOC107855741 | XM_016700750.1 | 0 | 832.79 | 91.36 | 0.47 |
| annuum | | | (SEQ ID NO: 10) | (SEQ ID NO: 38) | | | | |
| Chenopodium | quinoa | 110709217 | LOC110709217 | XM_021887429.1 | 0 | 876.70 | 98.36 | 0 |
| quinoa | | | (SEQ ID NO: 11) | (SEQ ID NO: 39) | | | | |
| | | 110712623 | LOC110712623 | XM_021891097.1 | 0 | 875.93 | 98.13 | 0 |
| | | | (SEQ ID NO: 12) | (SEQ ID NO: 40) | | | | |
| Citrullus | watermelon | Cla97Chr05 | Cla97C05G107810 | Cla97C05G107810.1 | 0 | 849.35 | 93.69 | 0 |
| lanatus | | | (SEQ ID NO: 13) | (SEQ ID NO: 41) | | | | |
| Cucurbita | winter squash | 111477743 | LOC111477743 | XM_023121629.1 | 0 | 848.58 | 93.69 | 0 |
| maxima | | | (SEQ ID NO: 16) | (SEQ ID NO: 44) | | | | |
| Cucumis | melon | 103488980 | LOC103488980 | XM_008447952.2 | 0 | 847.81 | 93.46 | 0 |
| melo | | | (SEQ ID NO: 14) | (SEQ ID NO: 42) | | | | |
| Cucurbita | summer | 111804264 | LOC111804264 | XM_023689006.1 | 0 | 848.58 | 93.69 | 0 |
| pepo subsp. | squash, | | (SEQ ID NO: 17) | (SEQ ID NO: 45) | | | | |
| pepo | zucchini | | | | | | | |
| Cucumis | cucumber | 101219697 | LOC101219697 | XM_004135216.3 | 0 | 845.88 | 93.22 | 0 |
| sativus | | | (SEQ ID NO: 15) | (SEQ ID NO: 43) | | | | |
| Daucus | carrot | 108202955 | LOC108202955 | XM_017371598.1 | 0 | 832.40 | 91.12 | 0 |
| carota | | | (SEQ ID NO: 18) | (SEQ ID NO: 46) | | | | |
| subsp. | | | | | | | | |
| sativus | | | | | | | | |
| Eruca | rocket | PAC: 39943752 | Eruve.3560s0005 | Eruve.3560s0005.1 | 0 | 830.09 | 90.19 | 0 |
| vesicaria | | | (SEQ ID NO: 19) | (SEQ ID NO: 47) | | | | |
| | | PAC: 39892663 | Eruve.1325s0030 | Eruve.1325s0030.1 | 0 | 830.09 | 90.19 | 0 |
| | | | (SEQ ID NO: 20) | (SEQ ID NO: 48) | | | | |
| Glycine max | soybean | 100789477 | LOC100789477 | XM_003551989.4 | 0 | 843.96 | 92.29 | 0 |
| | | | (SEQ ID NO: 21) | (SEQ ID NO: 49) | | | | |
| | | 100801068 | LOC100801068 | XM_003531942.3 | 0 | 843.96 | 92.29 | 0 |
| | | | (SEQ ID NO: 22) | (SEQ ID NO: 50) | | | | |
| Helianthus | sunflower | 110895091 | LOC110895091 | XM_022142374.2 | 0 | 847.81 | 93.46 | 0 |
| annuus | | | (SEQ ID NO: 23) | (SEQ ID NO: 51) | | | | |
| | | | | XM_022142375.2 | 0 | 847.81 | 93.46 | 0 |
| | | | | (SEQ ID NO: 52) | | | | |
| Humulus | common hop | FID: 1178425274 | HL.Tea.v1.0.G009494 | HL.Tea.v1.0.G009494.1 | 0 | 811.99 | 84.42′ | 7.36 |
| lupulus var. | | | (SEQ ID NO: 24) | (SEQ ID NO: 53) | | | | |
| lupulus | | | | | | | | |
| Lactuca | lettuce | 111882293 | LOC111882293 | XM_023878647.1 | 0 | 840.49 | 92.29 | 0 |
| sativa | | | (SEQ ID NO: 25) | (SEQ ID NO: 54) | | | | |
| Nicotiana | tobacco | 107768126 | LOC107768126 | XM_016587229.1 | 0 | 833.56 | 91.59 | 0.47 |
| tabacum | | | (SEQ ID NO: 26) | (SEQ ID NO: 55) | | | | |
| Ocimum | basil | FID: 3233744503 | 481.g272 | 481.g272.t1 | 0 | 846.65 | 93.69 | 0 |
| basilicum | | | (SEQ ID NO: 27) | (SEQ ID NO: 56) | | | | |
| var. | | FID: 3233710071 | 385868.g31 | 385868.g31.t1 | 0 | 848.20 | 93.69 | 0 |
| Genoveser | | | (SEQ ID NO: 28) | (SEQ ID NO: 57) | | | | |
| Phaseolus | common bean | 18634066 | PHAVU_003G063300g | XM_007153765.1 | 0 | 840.11 | 92.06 | 0 |
| vulgaris | | | (SEQ ID NO: 29) | (SEQ ID NO: 58) | | | | |
| Solanum | tomato | 101264626 | LOC101264626 | XM_004239071.4 | 0 | 827.78 | 97.20 | 0 |
| lycopersicum | | | (SEQ ID NO: 30) | (SEQ ID NO: 59) | | | | |
| Solanum | potato | 102586521 | LOC102586521 | XM_006357553.2 | 0 | 827.78 | 90.89 | 0.47 |
| tuberosum | | | (SEQ ID NO: 31) | (SEQ ID NO: 60) | | | | |
| | | 102589986 | LOC102589986 | XM_006366304.2 | 0 | 825.85 | 90.40 | 0.47 |
| | | | (SEQ ID NO: 32) | (SEQ ID NO: 61) | | | | |
| Vitis | grape | PAC: 38047563 | VIT_200s0225g00220 | VIT_200s0225g00220.1 | 0 | 844.00 | 93.00 | 0 |
| vinifera | | | (SEQ ID NO: 33) | (SEQ ID NO: 62) | | | | |
| Zea mays | maize | 103651285 | LOC103651285 | NM_001301599.1 | 0 | 822.39 | 90.21 | 0.23 |
| | | | (SEQ ID NO: 34) | (SEQ ID NO: 63) | | | | |
| Zea mays | maize | 103651285 | LOC103651285 | NM_001301599.1 | 0 | 822.39 | 90.21 | 0.23 |
| | | | (SEQ ID NO: 34) | (SEQ ID NO: 63) | | | | |

TABLE 3

| | | | | | E- | | % | |
|---|---|---|---|---|---|---|---|---|
| Organism | Common name | Gene ID | Locus | Transcript ID | value | Bit score | Identity | % Gaps |
| Spinacia | spinach | | Sp75 Spo10764 | (SEQ ID NO: 97) | | | 100.00 | |
| oleracea | | | (SEQ ID NO: 96) | | | | | |
| Beta vulgaris | beet, swiss | 104897370 | LOC104897370 | XM_010684230.2 | 0 | 1023.85 | 78.73 | 0.15 |
| subsp. | chard | | (SEQ ID NO: 99) | (SEQ ID NO: 122) | | | | |
| vulgaris | | | | | | | | |
| Brassica | rapeseed | | RID46578 | | 0 | 582.02 | 51.78 | 0.51 |
| napus | | | | | | | | |
| Brassica | wild cabbage | 106343209 | LOC106343209 | XM_013782361.1 | 0 | 605.13 | 49.84 | 0.16 |
| oleracea var. | | | (SEQ ID NO: 100) | (SEQ ID NO: 123) | | | | |
| oleracea | | | | | | | | |
| Capsicum | pepper | 107852979 | LOC107852979 | XM_016698013.1 | 0 | 635.95 | 52.02 | 0.48 |
| annuum | | | (SEQ ID NO: 101) | (SEQ ID NO: 124) | | | | |
| Chenopodium | quinoa | 110708874 | LOC110708874 | XM_021887098.1 | 0 | 1061.98 | 84.4 | 0 |
| quinoa | | | (SEQ ID NO: 102) | (SEQ ID NO: 125) | | | | |
| Citrullus | watermelon | Cla97Chr08 | Cla97C08G161690 | Cla97C08G161690.1 | | | | |
| lanatus | | | (SEQ ID NO: 103) | (SEQ ID NO: 126) | | | | |
| Cucurbita | winter squash | 111492496 | LOC111492496 | (SEQ ID NO: 127) | 0 | 657.52 | 53.22 | 0.47 |
| maxima | | | (SEQ ID NO: 104) | | | | | |
| Cucumis | melon | 107990292 | LOC107990292 | XM_017044416.1 | 0 | 621.70 | 51.86 | 0.32 |
| melo | | | (SEQ ID NO: 105) | (SEQ ID NO: 128) | | | | |
| Cucurbita | summer | 111805017 | LOC111805017 | XM_023689874.1 | 0 | 659.06 | 53.06 | 0.47 |
| pepo subsp. | squash, | | (SEQ ID NO: 106) | (SEQ ID NO: 129) | | | | |
| pepo | zucchini | | | | | | | |
| Cucumis | cucumber | 105434631 | LOC105434631 | XM_023141844.1 | 0 | 657.52 | 53.22 | 0.47 |
| sativus | | | (SEQ ID NO: 107) | (SEQ ID NO: 130) | | | | |
| Daucus | carrot | 108201762 | LOC108201762 | XM_017370050.1 | 0 | 639.03 | 52.77 | 0 |
| carota | | | (SEQ ID NO: 108) | (SEQ ID NO: 131) | | | | |
| subsp. | | | | | | | | |
| sativus | | | | | | | | |
| Eruca | rocket | PAC: 39943401 | Eruve.1454s0028 | Eruve.1454s0028.1 | 0 | 539.26 | 51.40 | 0.37 |
| vesicaria | | | (SEQ ID NO: 109) | (SEQ ID NO: 132) | | | | |
| | | PAC: 39898490 | Eruve.0510s0014 | Eruve.0510s0014.1 | 0 | 510.76 | 50.09 | 0.37 |
| | | | (SEQ ID NO: 110) | (SEQ ID NO: 133) | | | | |
| Glycine max | soybean | 102669506 | LOC102669506 | XM_006585882.4 | 0 | 592.81 | 50.56 | 0.8 |
| | | | (SEQ ID NO: 111) | (SEQ ID NO: 134) | | | | |
| Helianthus | sunflower | 110922025 | LOC110922025 | XM_022166353.2 | 0 | 661.37 | 52.75 | 0.31 |
| annuus | | | (SEQ ID NO: 112) | (SEQ ID NO: 135) | | | | |
| Humulus | common hop | FID: 1178471116 | (SEQ ID NO: 113) | HL.Tea.v1.0.G034437.1 | 7.15E−03 | 38.12 | 20.77 | 0.00 |
| lupulus var. | | | | (SEQ ID NO: 136) | | | | |
| lupulus | | | | | | | | |
| Lactuca | lettuce | 111896741 | LOC111896741 | XM_023892727.1 | 7.21E−143 | 431.80 | 51.77 | 0.44 |
| sativa | | | (SEQ ID NO: 114) | (SEQ ID NO: 137) | | | | |
| Nicotiana | tobacco | 107813009 | LOC107813009 | XM_016638208.1 | 0 | 652.51 | 51.23 | 0.46 |
| tabacum | | | (SEQ ID NO: 115) | (SEQ ID NO: 138) | | | | |
| Ocimum | basil | FID: 3233400634 | 381471.97 | 381471.g7.t1 | 7.76E−171 | 513.46 | 49.19 | 1.62 |
| basilicum | | | (SEQ ID NO: 116) | (SEQ ID NO: 139) | | | | |
| var. | | | | | | | | |
| Genoveser | | | | | | | | |
| Phaseolus | common bean | 18634050 | PHAVU_003G061700g | XM_007153685.1 | 0 | 598.59 | 50.40 | 0.65 |
| vulgaris | | | (SEQ ID NO: 117) | (SEQ ID NO: 140) | | | | |
| Solanum | tomato | 109120124 | LOC109120124 | XM_019213519.2 | 3.35E−133 | 403.29 | 55.17 | 0 |
| lycopersicum | | | (SEQ ID NO: 118) | (SEQ ID NO: 141) | | | | |
| Solanum | potato | 102606213 | LOC102606213 | XM_006350212.2 | 0 | 621.70 | 51.95 | 0 |
| tuberosum | | | (SEQ ID NO: 119) | (SEQ ID NO: 142) | | | | |
| Vitis | grape | 109122670 | LOC109122670 | XM_019220132.1 | 0 | 690.65 | 57.37 | 0 |
| vinifera | | | (SEQ ID NO: 120) | (SEQ ID NO: 143) | | | | |
| Zea mays | maize | 100282858 | LOC100282858 | NM_001155764.1 | 8.88E−136 | 428.33 | 41.84 | 1.22 |
| | | | (SEQ ID NO: 121) | (SEQ ID NO: 144) | | | | |

TABLE 4

| Organism | Common name | Gene ID | Locus | Transcript ID | E-value | Bit score | % Identity | % Gaps |
|---|---|---|---|---|---|---|---|---|
| Spinacia | spinach | PAC: 45135072 | Spov3_chr4.04649 | (SEQ ID NO: 173) | | | 100.00 | |
| oleracea | | | (SEQ ID NO: 172) | | | | | |
| Beta | beet, swiss | 104908660 | LOC104908660 | XM_010697794.2 | 0 | 969.92 | 78.80 | 5 |
| vulgaris | chard | | (SEQ ID NO: 175) | (SEQ ID NO: 197) | | | | |
| subsp. | | | | | | | | |
| vulgaris | | | | | | | | |
| Brassica | rapeseed | 106409344 | LOC106409344 | XM_022706436.1 | 6.61E−58 | 222.25 | 47.2 | 11.71 |
| napus | | | (SEQ ID NO: 176) | (SEQ ID NO: 198) | | | | |
| Brassica | wild cabbage | 106306990 | LOC106306990 | XM_013743817.1 | 0 | 226.87 | 47.61 | 11.41 |
| oleracea | | | (SEQ ID NO: 177) | (SEQ ID NO: 199) | | | | |
| var. | | | | | | | | |
| oleracea | | | | | | | | |
| Capsicum | pepper | 107840907 | LOC107840907 | XM_016684857.1 | 5.94E−84 | 295.82 | 50.42 | 11.52 |
| annuum | | | (SEQ ID NO: 178) | (SEQ ID NO: 200) | | | | |
| Chenopodium | quinoa | 110726726 | LOC110726726 | XM_021906204.1 | 0 | 949.12 | 75.8 | 5.43 |
| quinoa | | | (SEQ ID NO: 179) | (SEQ ID NO: 201) | | | | |
| Citrullus | watermelon | na | Cla97C11G216720 | Cla97C11G216720.1 | 2.96E−31 | 132.88 | 25.4 | 13.74 |
| lanatus | | | (SEQ ID NO: 180) | (SEQ ID NO: 202) | | | | |
| Cucurbita | winter | 111492444 | LOC111492444 | XM_023141784.1 | 3.62E−47 | 189.5 | 47 | 14 |
| maxima | squash | | (SEQ ID NO: 183) | (SEQ ID NO: 205) | | | | |
| Cucumis | melon | na | MELO3C008693 | MELO3C008693.2.1 | 1.27E−95 | 144.05 | 26.81 | 13.47 |
| melo | | | (SEQ ID NO: 181) | (SEQ ID NO: 203) | | | | |
| Cucurbita | summer | Cp4.1LG16 | Cp4.1LG16g01740 | Cp4.1LG16g01740.1 | 1.30E−30 | 189.5 | 47 | 14 |
| pepo | squash, | | (SEQ ID NO: 184) | (SEQ ID NO: 206) | | | | |
| | zucchini | | | | | | | |
| Cucumis | cucumber | na | CsGy2G008360 | CsGy2G008360.1 | 1.71E−33 | 140.2 | 26.5 | 10.16 |
| sativus | | | (SEQ ID NO: 182) | (SEQ ID NO: 204) | | | | |
| Daucus | carrot | 108222647 | LOC108222647 | XM_017396535.1 | 0 | 202.22 | 46.30 | 13.55 |
| carota | | | (SEQ ID NO: 186) | (SEQ ID NO: 208) | | | | |
| subsp. | | | | | | | | |
| sativus | | | | | | | | |
| Eruca | rocket | PAC: 39900680 | Eruve.2647s0012 | Eruve.2647s0012.1 | 0.02 | 38.51 | 26.05 | 9.24 |
| vesicaria | | | (SEQ ID NO: 187) | (SEQ ID NO: 209) | | | | |
| Glycine | soybean | 114378323 | LOC114378323 | XM_028336895.1 | 1.98E−83 | 294.66 | 50.36 | 16.07 |
| max | | | (SEQ ID NO: 188) | (SEQ ID NO: 210) | | | | |
| Helianthus | sunflower | 110888041 | LOC110888041 | XM_022135586.1 | 4.14E−46 | 186.42 | 46.5 | 8.88 |
| annuus | | | (SEQ ID NO: 189) | (SEQ ID NO: 211) | | | | |
| Lactuca | lettuce | 111884527 | LOC111884527 | XM_023880841.1 | 2.41E−90 | 313.15 | 53.21 | 10.74 |
| sativa | | | (SEQ ID NO: 190) | (SEQ ID NO: 212) | | | | |
| Nicotiana | tobacco | 107783800 | LOC107783800 | XM_016604819.1 | 3.06E−82 | 289.66 | 52.15 | 12.17 |
| tabacum | | | (SEQ ID NO: 191) | (SEQ ID NO: 213) | | | | |
| Ocimum | basil | FID: 3233781621 | 544.g114 | 544.g114.t1 | 5.19E−95 | 317.77 | 31.68 | 8.78 |
| basilicum | | | (SEQ ID NO: 192) | (SEQ ID NO: 214) | | | | |
| var. | | | | | | | | |
| Genoveser | | | | | | | | |
| Phaseolus | common | 18630497 | PHAVU_005G087100g | XM_007149584.1 | 4.76E−83 | 293.89 | 48.99 | 16.61 |
| vulgaris | bean | | (SEQ ID NO: 193) | (SEQ ID NO: 215) | | | | |
| Solanum | tomato | 101244633 | LOC101244633 | XM_004249666.4 | 1.52E−85 | 300.83 | 50.12 | 14.85 |
| lycopersicum | | | (SEQ ID NO: 185) | (SEQ ID NO: 207) | | | | |
| Solanum | potato | 102583217 | LOC102583217 | XM_015310721.1 | 7.37E−73 | 264.62 | 48.9 | 9.81 |
| tuberosum | | | (SEQ ID NO: 194) | (SEQ ID NO: 216) | | | | |
| Vitis | grape | 100258875 | LOC100258875 | XM_002283378.3 | 1.17E−94 | 325.87 | 51.35 | 12.61 |
| vinifera | | | (SEQ ID NO: 195) | (SEQ ID NO: 217) | | | | |
| Zea mays | maize | 103651126 | LOC103651126 | XM_008676725.3 | 5.47E−47 | 188.73 | 46.88 | 14 |
| | | | (SEQ ID NO: 196) | (SEQ ID NO: 218) | | | | |

TABLE 5

| Candidate Gene | Gene model | Identity % | Position | Genomic | cDNA | Protein |
|---|---|---|---|---|---|---|
| | Sunflower (XRQ2) | | | | | |
| Sp75 Spo10764 | HanXRQr2_Chr17g0787021 | 52.7 | HanXRQChr17: 15291060 . . . 15293054 | SEQ ID | SEQ ID | SEQ ID |
| | | | | NO: 317 | NO: 322 | NO: 327 |
| Spov3_chr4.04752 | HanXRQr2_Chr12g0544841 | 93 | HanXRQChr12: 61292169 . . . 61300151 | SEQ ID | SEQ ID | SEQ ID |
| | | | | NO: 258 | NO: 278 | NO: 300 |
| Spov3_chr4.04752 | HanXRQr2_Chr12g0563761 | 90 | HanXRQChr12: 158512420 . . . 158519768 | SEQ ID | SEQ ID | SEQ ID |
| | | | | NO: 259 | NO: 279 | NO: 301 |
| Spov3_chr4.04752 | HanXRQr2_Chr02g0051381 | 77 | HanXRQChr02: 21014549 . . . 21018351 | SEQ ID | SEQ ID | SEQ ID |
| | | | | NO: 260 | NO: 280 | NO: 302 |
| | Sorghum (BTx623v3) | | | | | |
| Sp75 Spo10764 | Sobic.010G255800.2 | 41% | Chr10: 59424506 . . . 59427930 | SEQ ID | SEQ ID | SEQ ID |
| | | | | NO: 318 | NO: 323 | NO: 328 |
| Spov3_chr4.04752 | Sobic.003G270600.1 | 90.20% | Chr03: 60727143 . . . 60735273 | SEQ ID | SEQ ID | SEQ ID |
| | | | | NO: 261 | NO: 281 | NO: 303 |
| | Rapeseed (Darmorv10) | | | | | |
| Sp75 Spo10764 | A03p36200.1_BnaDAR-T1 | 33.10% | A03: 16562227 . . . 16565322 | SEQ ID | SEQ ID | SEQ ID |
| | | | | NO: 319 | NO: 324 | NO: 329 |
| Spov3_chr4.04752 | A09p64610.1_BnaDAR-T1 | 90.70% | A09: 51206504 . . . 51209206 | SEQ ID | SEQ ID | SEQ ID |
| | | | | NO: 262 | NO: 282 | NO: 304 |
| Spov3_chr4.04752 | C08p48590.1_BnaDAR-T1 | 90.40% | C08: 38839812 . . . 38842948 | SEQ ID | SEQ ID | SEQ ID |
| | | | | NO: 263 | NO: 283 | NO: 305 |
| Spov3_chr4.04752 | C05p09110.1_BnaDAR-T1 | 90.20% | C05: 4627469 . . . 4630182 | SEQ ID | SEQ ID | SEQ ID |
| | | | | NO: 264 | NO: 284 | NO: 306 |
| Spov3_chr4.04752 | A01p29530.1_BnaDAR-T1 | 90.40% | A01: 20492291 . . . 20495341 | SEQ ID | SEQ ID | SEQ ID |
| | | | | NO: 265 | NO: 285 | NO: 307 |
| Spov3_chr4.04752 | A06p07560.1_BnaDAR-T1 | 90% | A06: 4237812 . . . 4240747 | SEQ ID | SEQ ID | SEQ ID |
| | | | | NO: 266 | NO: 286 | NO: 308 |
| Spov3_chr4.04752 | C01p37360.1_BnaDAR-T1 | 89.70% | C01: 32908478 . . . 32911143 | SEQ ID | SEQ ID | SEQ ID |
| | | | | NO: 267 | NO: 287 | NO: 309 |
| | Maize (B73 AGPv4) | | | | | |
| Spov3_chr4.04752 | Zm00001d043726_T006 | 90.20% | chr3: 208445923 . . . 208450685 | SEQ ID | SEQ ID | SEQ ID |
| | | | | NO: 268 | NO: 288 | NO: 310 |
| Spov3_chr4.04752 | Zm00001d043726_T001 | 90.20% | chr3: 208445923 . . . 208450685 | SEQ ID | SEQ ID | SEQ ID |
| | | | | NO: 268 | NO: 289 | NO: 311 |
| Spov3_chr4.04752 | Zm00001d043726_T002 | 87.40% | chr3: 208445923 . . . 208450685 | SEQ ID | SEQ ID | SEQ ID |
| | | | | NO: 268 | NO: 290 | NO: 312 |
| Spov3_chr4.04752 | Zm00001d011676_T002 | 77.30% | chr8: 158309275 . . . 158313766 | SEQ ID | SEQ ID | SEQ ID |
| | | | | NO: 269 | NO: 291 | NO: 313 |
| Spov3_chr4.04752 | Zm00001d011676_T001 | 77.30% | chr8: 158309275 . . . 158313766 | SEQ ID | SEQ ID | SEQ ID |
| | | | | NO: 269 | NO: 292 | NO: 314 |

TABLE 6

| Candidate Gene | Organism | Gene Model | Genomic | cDNA | Protein |
|---|---|---|---|---|---|
| Spov3_chr4.04752 | Beet, Swiss chard | Bv6_148070_ijzy.t1 | SEQ ID NO: 270 | SEQ ID NO: 293 | SEQ ID NO: 315 |
| | (Beta vulgaris) | | | | |
| Sp75 Spo10764 | | Bv6_148650_nahx.t1 | SEQ ID NO: 320 | SEQ ID NO: 325 | SEQ ID NO: 330 |
| Spov3_chr4.04649 | | Bv7_177740_zcug.t1 | SEQ ID NO: 331 | SEQ ID NO: 332 | SEQ ID NO: 333 |

### Altering the Activity of a Protein Encoded by an S-Gene

In certain embodiments the invention may include modulation of the S-gene to alter, induce, reduce, or eliminate the activity of a protein encoded by an S-gene, perhaps during certain developmental stages or tissues etc., by transforming a plant cell with an expression cassette that expresses a polynucleotide that alters the expression of the protein. The polynucleotide may alter the expression of the protein directly, by influencing transcription or translation of the messenger RNA, or indirectly, by encoding a polypeptide that induces, reduces, or inhibits transcription or translation. Methods for altering, inducing, reducing, inhibiting or eliminating the expression of a gene in a plant are well known in the art, and any such method may be used in the present invention to alter the expression of a protein encoded by an S-gene. Many methods may be used to reduce or eliminate or otherwise alter the activity of a protein encoded by an S-gene. In addition, more than one method may be used to alter the activity of a protein encoded by an S-gene.

### 1. Polynucleotide-Based Methods:

In some embodiments of the present invention, a plant is transformed with an expression cassette that is capable of expressing a polynucleotide that inhibits the expression of a protein encoded by an S-gene of the invention. The term “expression” as used herein refers to the biosynthesis of a gene product, including the transcription and/or translation of said gene product. For example, for the purposes of the present invention, an expression cassette capable of expressing a polynucleotide that inhibits the expression of at least one protein encoded by an S-gene is an expression cassette capable of producing an RNA molecule that inhibits the transcription and/or translation of at least one protein encoded by an S-gene of the invention. The “expression” or “production” of a protein or polypeptide from a DNA molecule refers to the transcription and translation of the coding sequence to produce the protein or polypeptide, while the “expression” or “production” of a protein or polypeptide from an RNA molecule refers to the translation of the RNA coding sequence to produce the protein or polypeptide.

Examples of polynucleotides that inhibit the expression of a protein encoded by an S-gene are given below.

i. Sense Suppression/Cosuppression

In some embodiments of the invention, inhibition of the expression of a protein encoded by an S-gene may be obtained by sense suppression or cosuppression. For cosuppression, an expression cassette is designed to express an RNA molecule corresponding to all or part of a messenger RNA encoding a protein in the “sense” orientation. Over expression of the RNA molecule can result in reduced expression of the native gene. Accordingly, multiple plant lines transformed with the cosuppression expression cassette are screened to identify those that show the greatest inhibition of protein expression.

The polynucleotide used for cosuppression may correspond to all or part of the sequence encoding the protein, all or part of the 5′ and/or 3′ untranslated region of a transcript, or all or part of both the coding sequence and the untranslated regions of a transcript encoding a protein. In some embodiments where the polynucleotide comprises all or part of the coding region for the protein, the expression cassette is designed to eliminate the start codon of the polynucleotide so that no protein product will be translated.

Cosuppression may be used to inhibit the expression of plant genes to produce plants having undetectable protein levels for the proteins encoded by these genes. See, for example, Broin, et al., (2002) Plant Cell 14:1417-1432. Cosuppression may also be used to inhibit the expression of multiple proteins in the same plant. See, for example, U.S. Pat. No. 5,942,657. Methods for using cosuppression to inhibit the expression of endogenous genes in plants are described in Flavell, et al., (1994) Proc. Natl. Acad. Sci. USA 91:3490-3496; Jorgensen, et al., (1996) Plant Mol. Biol. 31:957-973; Johansen and Carrington, (2001) Plant Physiol. 126:930-938; Broin, et al., (2002) Plant Cell 14:1417-1432; Stoutjesdijk, et al., (2002) Plant Physiol. 129:1723-1731; Yu, et al., (2003) Phytochemistry 63:753-763; and U.S. Pat. Nos. 5,034,323, 5,283,184, and 5,942,657. The efficiency of cosuppression may be increased by including a poly-dT region in the expression cassette at a position 3′ to the sense sequence and 5′ of the polyadenylation signal. See, U.S. Patent Publication No. 20020048814. Typically, such a nucleotide sequence has substantial sequence identity to the sequence of the transcript of the endogenous gene, optimally greater than about 65% sequence identity, more optimally greater than about 85% sequence identity, most optimally greater than about 95% sequence identity. See U.S. Pat. Nos. 5,283,184 and 5,034,323.

ii. Antisense Suppression

In some embodiments of the invention, inhibition of the expression of a protein encoded by an S-gene may be obtained by antisense suppression. For antisense suppression, the expression cassette is designed to express an RNA molecule complementary to all or part of a messenger RNA encoding the protein. Over expression of the antisense RNA molecule can result in reduced expression of the native gene. Accordingly, multiple plant lines transformed with the antisense suppression expression cassette are screened to identify those that show the greatest inhibition of protein expression.

The polynucleotide for use in antisense suppression may correspond to all or part of the complement of the sequence encoding the protein, all or part of the complement of the 5′ and/or 3′ untranslated region of the transcript, or all or part of the complement of both the coding sequence and the untranslated regions of a transcript encoding the protein. In addition, the antisense polynucleotide may be fully complementary (i.e., 100% identical to the complement of the target sequence) or partially complementary (i.e., less than 100% identical to the complement of the target sequence) to the target sequence. Antisense suppression may be used to inhibit the expression of multiple proteins in the same plant. See, for example, U.S. Pat. No. 5,942,657. Furthermore, portions of the antisense nucleotides may be used to disrupt the expression of the target gene. Generally, sequences of at least 50 nucleotides, 100 nucleotides, 200 nucleotides, 300, 400, 450, 500, 550, or greater may be used. Methods for using antisense suppression to inhibit the expression of endogenous genes in plants are described, for example, in Liu, et al., (2002) Plant Physiol. 129:1732-1743 and U.S. Pat. Nos. 5,759,829 and 5,942,657. Efficiency of antisense suppression may be increased by including a poly-dT region in the expression cassette at a position 3′ to the antisense sequence and 5′ of the polyadenylation signal. See, U.S. Patent Publication No. 20020048814.

iii. Double-Stranded RNA Interference

In some embodiments of the invention, inhibition of the expression of a protein encoded by an S-gene may be obtained by double-stranded RNA (dsRNA) interference. For dsRNA interference, a sense RNA molecule like that described above for cosuppression and an antisense RNA molecule that is fully or partially complementary to the sense RNA molecule are expressed in the same cell, resulting in inhibition of the expression of the corresponding endogenous messenger RNA.

Expression of the sense and antisense molecules can be accomplished by designing the expression cassette to comprise both a sense sequence and an antisense sequence. Alternatively, separate expression cassettes may be used for the sense and antisense sequences. Multiple plant lines transformed with the dsRNA interference expression cassette or expression cassettes are then screened to identify plant lines that show the greatest inhibition of protein expression. Methods for using dsRNA interference to inhibit the expression of endogenous plant genes are described in Waterhouse, et al., (1998) Proc. Natl. Acad. Sci. USA 95:13959-13964, Liu, et al., (2002) Plant Physiol. 129:1732-1743, and WO 99/49029, WO 99/53050, WO 99/61631, and WO 00/49035.

iv. Hairpin RNA Interference and Intron-Containing Hairpin RNA Interference

In some embodiments of the invention, inhibition of the expression of a protein encoded by an S-gene may be obtained by hairpin RNA (hpRNA) interference or intron-containing hairpin RNA (ihpRNA) interference. These methods are highly efficient at inhibiting the expression of endogenous genes. See, Waterhouse and Helliwell, (2003) Nat. Rev. Genet. 4:29-38 and the references cited therein.

For hpRNA interference, the expression cassette is designed to express an RNA molecule that hybridizes with itself to form a hairpin structure that comprises a single-stranded loop region and a base-paired stem. The base-paired stem region comprises a sense sequence corresponding to all or part of the endogenous messenger RNA encoding the gene whose expression is to be inhibited, and an antisense sequence that is fully or partially complementary to the sense sequence. Alternatively, the base-paired stem region may correspond to a portion of a promoter sequence controlling expression of the gene to be inhibited. Thus, the base-paired stem region of the molecule generally determines the specificity of the RNA interference. hpRNA molecules are highly efficient at inhibiting the expression of endogenous genes, and the RNA interference they induce is inherited by subsequent generations of plants. See, for example, Chuang and Meyerowitz, (2000) Proc. Natl. Acad. Sci. USA 97:4985-4990; Stoutjesdijk, et al., (2002) Plant Physiol. 129:1723-1731; and Waterhouse and Helliwell, (2003) Nat. Rev. Genet. 4:29-38. Methods for using hpRNA interference to inhibit or silence the expression of genes are described, for example, in Chuang and Meyerowitz, (2000) Proc. Natl. Acad. Sci. USA 97:4985-4990; Stoutjesdijk, et al., (2002) Plant Physiol. 129:1723-1731; Waterhouse and Helliwell, (2003) Nat. Rev. Genet. 4:29-38; Pandolfini et al., BMC Biotechnology 3:7, and U.S. Patent Publication No. 2003/0175965. A transient assay for the efficiency of hpRNA constructs to silence gene expression in vivo has been described by Panstruga, et al., (2003) Mol. Biol. Rep. 30:135-140.

For ihpRNA, the interfering molecules have the same general structure as for hpRNA, but the RNA molecule additionally comprises an intron that is capable of being spliced in the cell in which the ihpRNA is expressed. The use of an intron minimizes the size of the loop in the hairpin RNA molecule following splicing, and this increases the efficiency of interference. See, for example, Smith, et al., (2000) Nature 407:319-320. In fact, Smith, et al., show 100% suppression of endogenous gene expression using ihpRNA-mediated interference. Methods for using ihpRNA interference to inhibit the expression of endogenous plant genes are described, for example, in Smith, et al., (2000) Nature 407:319-320; Wesley, et al., (2001) Plant J. 27:581-590; Wang and Waterhouse, (2001) Curr. Opin. Plant Biol. 5:146-150; Waterhouse and Helliwell, (2003) Nat. Rev. Genet. 4:29-38; Helliwell and Waterhouse, (2003) Methods 30:289-295, and U.S. Patent Publication No. 2003/0180945.

The expression cassette for hpRNA interference may also be designed such that the sense sequence and the antisense sequence do not correspond to an endogenous RNA. In this embodiment, the sense and antisense sequence flank a loop sequence that comprises a nucleotide sequence corresponding to all or part of the endogenous messenger RNA of the target gene. Thus, it is the loop region that determines the specificity of the RNA interference. See, for example, WO 02/00904; Mette, et al., (2000) EMBO J 19:5194-5201; Matzke, et al., (2001) Curr. Opin. Genet. Devel. 11:221-227; Scheid, et al., (2002) Proc. Natl. Acad. Sci., USA 99:13659-13662; Aufsaftz, et al., (2002) Proc. Nat'l. Acad. Sci. 99 (4): 16499-16506; Sijen, et al., Curr. Biol. (2001) 11:436-440).

v. Amplicon-Mediated Interference

Amplicon expression cassettes comprise a plant virus-derived sequence that contains all or part of the target gene but generally not all of the genes of the native virus. The viral sequences present in the transcription product of the expression cassette allow the transcription product to direct its own replication. The transcripts produced by the amplicon may be either sense or antisense relative to the target sequence (i.e., the messenger RNA). Methods of using amplicons to inhibit the expression of endogenous plant genes are described, for example, in Angell and Baulcombe, (1997) EMBO J. 16:3675-3684, Angell and Baulcombe, (1999) Plant J. 20:357-362, and U.S. Pat. No. 6,635,805.

vi. Ribozymes

In some embodiments, the polynucleotide expressed by the expression cassette of the invention is catalytic RNA or has ribozyme activity specific for the messenger RNA of the protein. Thus, the polynucleotide causes the degradation of the endogenous messenger RNA, resulting in reduced expression of a protein encoded by an S-gene. This method is described, for example, in U.S. Pat. No. 4,987,071.

vii. Small Interfering RNA or Micro RNA

In some embodiments of the invention, inhibition of the expression a protein encoded by an S-gene may be obtained by RNA interference by expression of a gene encoding a micro RNA (miRNA). miRNAs are regulatory agents consisting of about 22 ribonucleotides. miRNA are highly efficient at inhibiting the expression of endogenous genes. See, for example Javier, et al., (2003) Nature 425:257-263.

For miRNA interference, the expression cassette is designed to express an RNA molecule that is modeled on an endogenous miRNA gene. The miRNA gene encodes an RNA that forms a hairpin structure containing a 22-nucleotide sequence that is complementary to another endogenous gene (target sequence). For suppression of S-gene expression, the 22-nucleotide sequence is selected from a transcript sequence and contains 22 nucleotides of said sequence in sense orientation and 21 nucleotides of a corresponding antisense sequence that is complementary to the sense sequence. miRNA molecules are highly efficient at inhibiting the expression of endogenous genes, and the RNA interference they induce is inherited by subsequent generations of plants.

### 2. Polypeptide-Based Inhibition of Gene Expression

In one embodiment, the polynucleotide encodes a zinc finger protein that binds to an S-gene encoding a polypeptide, resulting in reduced expression of the S-gene. In particular embodiments, the zinc finger protein binds to a regulatory region of an S-gene. In other embodiments, the zinc finger protein binds to a messenger RNA encoding a polypeptide and prevents its translation. Methods of selecting sites for targeting by zinc finger proteins have been described, for example, in U.S. Pat. No. 6,453,242, and methods for using zinc finger proteins to inhibit the expression of genes in plants are described, for example, in U.S. Patent Publication No. 2003/0037355.

### 3. Polypeptide-Based Inhibition of Protein Activity

In some embodiments of the invention, the polynucleotide encodes an antibody that binds to at least one polypeptide encoded by an S-gene, and reduces the activity of the polypeptide. In another embodiment, the binding of the antibody results in increased turnover of the antibody-polypeptide complex by cellular quality control mechanisms. The expression of antibodies in plant cells and the inhibition of molecular pathways by expression and binding of antibodies to proteins in plant cells are well known in the art. See, for example, Conrad and Sonnewald, (2003) Nature Biotech. 21:35-36.

### 4. Gene Disruption

In some embodiments of the present invention, the activity of a polypeptide encoded by an S-gene may be reduced or eliminated by disrupting the S-gene encoding the polypeptide. The S-gene encoding the polypeptide may be disrupted by any method known in the art. For example, in one embodiment, the gene is disrupted by transposon tagging. In another embodiment, the gene is disrupted by mutagenizing plants using random or targeted mutagenesis, and selecting for plants that have desired traits.

i. Transposon Tagging

In one embodiment of the invention, transposon tagging is used to reduce or eliminate the activity of one or more polypeptides encoded by an S-gene. Transposon tagging comprises inserting a transposon within an endogenous S-gene to reduce or eliminate expression of the polypeptide.

In this embodiment, the expression of one or more polypeptides encoded by an S-gene is reduced or eliminated by inserting a transposon within a regulatory region or coding region of the S-gene encoding the polypeptide. A transposon that is within an exon, intron, 5′ or 3′ untranslated sequence, a promoter, or any other regulatory sequence of an S-gene may be used to reduce or eliminate the expression and/or activity of the encoded polypeptide.

Methods for the transposon tagging of specific genes in plants are well known in the art. See, for example, Maes, et al., (1999) Trends Plant Sci. 4:90-96; Dharmapuri and Sonti, (1999) FEMS Microbiol. Lett. 179:53-59; Meissner, et al., (2000) Plant J. 22:265-274; Phogat, et al., (2000) J. Biosci. 25:57-63; Walbot, (2000) Curr. Opin. Plant Biol. 2:103-107; Gai, et al., (2000) Nucleic Acids Res. 28:94-96; Fitzmaurice, et al., (1999) Genetics 153:1919-1928). In addition, the TUSC process for selecting Mu insertions in selected genes has been described in Bensen, et al., (1995) Plant Cell 7:75-84; Mena, et al., (1996) Science 274:1537-1540; and U.S. Pat. No. 5,962,764.

ii. Mutant Plants with Reduced Activity

Additional methods for decreasing or eliminating the expression of endogenous genes in plants are also known in the art and can be similarly applied to the instant invention. These methods include other forms of mutagenesis, such as ethyl methanesulfonate-induced mutagenesis, deletion mutagenesis, and fast neutron deletion mutagenesis used in a reverse genetics sense (with PCR) to identify plant lines in which the endogenous gene has been deleted. For examples of these methods see, Ohshima, et al., (1998) Virology 243:472-481; Okubara, et al., (1994) Genetics 137:867-874; and Quesada, et al., (2000) Genetics 154:421-436. In addition, a fast and automatable method for screening for chemically induced mutations, TILLING (Targeting Induced Local Lesions In Genomes), using denaturing HPLC or selective endonuclease digestion of selected PCR products is also applicable to the instant invention. See, McCallum, et al., (2000) Nat. Biotechnol. 18:455-457.

Mutations that impact gene expression or that interfere with the function of the encoded protein are well known in the art. Insertional mutations in gene exons usually result in null-mutants. Mutations in conserved residues are particularly effective in inhibiting the activity of the encoded protein. Conserved residues of polypeptides suitable for mutagenesis with the goal to eliminate activity have been described. Such mutants can be isolated according to well-known procedures, and mutations in different loci can be stacked by genetic crossing. See, for example, Gruis, et al., (2002) Plant Cell 14:2863-2882.

In another embodiment of this invention, dominant mutants can be used to trigger RNA silencing due to gene inversion and recombination of a duplicated gene locus. See, for example, Kusaba, et al., (2003) Plant Cell 15:1455-1467.

The invention encompasses additional methods for altering (including reducing, inducing or eliminating) the activity of one or more polypeptides encoded by an S-gene. Examples of other methods for altering or mutating a genomic nucleotide sequence in a plant are known in the art and include, but are not limited to, the use of RNA: DNA vectors, RNA: DNA mutational vectors, RNA: DNA repair vectors, mixed-duplex oligonucleotides, self-complementary RNA: DNA oligonucleotides, and recombinogenic oligonucleobases. Such vectors and methods of use are known in the art. See, for example, U.S. Pat. Nos. 5,565,350; 5,731,181; 5,756,325; 5,760,012; 5,795,972; and 5,871,984. See also, WO 98/49350, WO 99/07865, WO 99/25821, and Beetham, et al., (1999) Proc. Natl. Acad. Sci. USA 96:8774-8778.

### Genome Editing and Induced Mutagenesis

In general, methods to modify or alter the host endogenous genomic DNA are available. This includes altering the host native DNA sequence or a pre-existing transgenic sequence including regulatory elements, coding and non-coding sequences. These methods are also useful in targeting nucleic acids to pre-engineered target recognition sequences in the genome. As an example, the genetically modified cell or plant described herein is generated using “custom” meganucleases produced to modify plant genomes (see, e.g., WO 2009/114321; Gao, et al., (2010) Plant Journal 1:176-187). Other site-directed engineering is through the use of zinc finger domain recognition coupled with the restriction properties of restriction enzyme. See, e.g., Urnov, et al., (2010) Nat Rev Genet. 11 (9): 636-46; Shukla, et al., (2009) Nature 459 (7245): 437-41.

“TILLING” or “Targeting Induced Local Lesions IN Genomics” refers to a mutagenesis technology useful to generate and/or identify and to eventually isolate mutagenised variants of a particular nucleic acid with modulated expression and/or activity (McCallum, et al., (2000), Plant Physiology 123:439-442; McCallum, et al., (2000) Nature Biotechnology 18:455-457 and Colbert, et al., (2001) Plant Physiology 126:480-484).

TILLING combines high density point mutations with rapid sensitive detection of the mutations. Typically, ethylmethanesulfonate (EMS) is used to mutagenize plant seed. EMS alkylates guanine, which typically leads to mispairing. For example, seeds are soaked in an about 10-20 mM solution of EMS for about 10 to 20 hours; the seeds are washed and then sown. The plants of this generation are known as M1. M1 plants are then self-fertilized. Mutations that are present in cells that form the reproductive tissues are inherited by the next generation (M2). Typically, M2 plants are screened for mutation in the desired gene and/or for specific phenotypes.

TILLING also allows selection of plants carrying mutant variants. These mutant variants may exhibit modified expression, either in strength or in location or in timing (if the mutations affect the promoter, for example). These mutant variants may exhibit higher or lower activity than that exhibited by the gene in its natural form. TILLING combines high-density mutagenesis with high-throughput screening methods. The steps typically followed in TILLING are: (a) EMS mutagenesis (Redei and Koncz, (1992) In Methods in Arabidopsis Research, Koncz, et al., eds. Singapore, World Scientific Publishing Co, pp. 16-82; Feldmann, et al., (1994) In Arabidopsis. Meyerowitz and Somerville, eds, Cold Spring Harbor Laboratory Press, Cold Spring Harbor, N.Y., pp 137-172; Lightner and Caspar, (1998) In Methods on Molecular Biology 82:91-104; Martinez-Zapater and Salinas, eds, Humana Press, Totowa, N.J.); (b) DNA preparation and pooling of individuals; (c) PCR amplification of a region of interest; (d) denaturation and annealing to allow formation of heteroduplexes; (e) DHPLC, where the presence of a heteroduplex in a pool is detected as an extra peak in the chromatogram; (f) identification of the mutant individual; and (g) sequencing of the mutant PCR product. Methods for TILLING are well known in the art (U.S. Pat. No. 8,071,840).

Other mutagenic methods can also be employed to introduce mutations in a disclosed gene. Methods for introducing genetic mutations into plant genes and selecting plants with desired traits are well known. For instance, seeds or other plant material can be treated with a mutagenic chemical substance, according to standard techniques. Such chemical substances include, but are not limited to, the following: diethyl sulfate, ethylene imine, and N-nitroso-N-ethylurea. Alternatively, ionizing radiation from sources such as X-rays or gamma rays can be used.

Another genome editing method that can be used according to the various aspects of the invention is CRISPR. The use of this technology in genome editing is well described in the art, for example in U.S. Pat. No. 8,697,359 and references cited herein. In short, CRISPR is a microbial nuclease system involved in defense against invading phages and plasmids. CRISPR loci in microbial hosts contain a combination of CRISPR-associated (Cas) genes as well as non-coding RNA elements capable of programming the specificity of the CRISPR-mediated nucleic acid cleavage (sgRNA). Three types (1-111) of CRISPR systems have been identified across a wide range of bacterial hosts. One key feature of each CRISPR locus is the presence of an array of repetitive sequences (direct repeats) interspaced by short stretches of non-repetitive sequences (spacers). The non-coding CRISPR array is transcribed and cleaved within direct repeats into short crRNAs containing individual spacer sequences, which direct Cas nucleases to the target site (protospacer). The Type II CRISPR is one of the most well characterized systems and carries out targeted DNA double-strand break in four sequential steps. First, two non-coding RNA, the pre-crRNA array and tracrRNA, are transcribed from the CRISPR locus. Second, tracrRNA hybridizes to the repeat regions of the pre-crRNA and mediates the processing of pre-crRNA into mature crRNAs containing individual spacer sequences. Third, the mature crRNA: tracrRNA complex directs Cas9 to the target DNA via Watson-Crick base-pairing between the spacer on the crRNA and the protospacer on the target DNA next to the protospacer adjacent motif (PAM), an additional requirement for target recognition. Finally, Cas9 mediates cleavage of target DNA to create a double-stranded break within the protospacer.

Cas9 is thus the hallmark protein of the type II CRISPR-Cas system, and a large monomeric DNA nuclease guided to a DNA target sequence adjacent to the PAM (protospacer adjacent motif) sequence motif by a complex of two noncoding RNAS: CRIPSR RNA (crRNA) and trans-activating crRNA (tracrRNA). The Cas9 protein contains two nuclease domains homologous to RuvC and HNH nucleases. The HNH nuclease domain cleaves the complementary DNA strand whereas the RuvC-like domain cleaves the non-complementary strand and, as a result, a blunt cut is introduced in the target DNA. Heterologous expression of Cas9 together with an sgRNA can introduce site-specific double strand breaks (DSBs) into genomic DNA of live cells from various organisms. For applications in eukaryotic organisms, codon optimized versions of Cas9, which is originally from the bacterium Streptococcus pyogenes, have been used.

The single guide RNA (sgRNA) is the second component of the CRISPR/Cas system that forms a complex with the Cas9 nuclease. sgRNA is a synthetic RNA chimera created by fusing crRNA with tracrRNA. The sgRNA guide sequence located at its 5′ end confers DNA target specificity. Therefore, by modifying the guide sequence, it is possible to create sgRNAs with different target specificities. The canonical length of the guide sequence is 20 bp. In plants, sgRNAs have been expressed using plant RNA polymerase III promoters, such as U6 and U3. Cas9 expression plasmids for use in the methods of the invention can be constructed as described in the art. Alternatively, Cas9 (or Cas12A) can be transferred to plant tissues as active proteins.

### Nucleotide Constructs, Expression Cassettes, and Vectors

The use of the term “nucleotide constructs” herein is not intended to limit the embodiments to nucleotide constructs comprising DNA. Those of ordinary skill in the art that nucleotide constructs, particularly polynucleotides and will recognize oligonucleotides composed of ribonucleotides and combinations of ribonucleotides and deoxyribonucleotides, may also be employed in the methods disclosed herein. The nucleotide constructs, nucleic acids, and nucleotide sequences of the embodiments additionally encompass all complementary forms of such constructs, molecules, and sequences. Further, the nucleotide constructs, nucleotide molecules, and nucleotide sequences of the embodiments encompass all nucleotide constructs, molecules, and sequences which can be employed in the methods of the embodiments for transforming plants including, but not limited to, those comprised of deoxyribonucleotides, ribonucleotides, and combinations thereof. Such deoxyribonucleotides and ribonucleotides include both naturally occurring molecules and synthetic analogues. The nucleotide constructs, nucleic acids, and nucleotide sequences of the embodiments also encompass all forms of nucleotide constructs including, but not limited to, single-stranded forms, double-stranded forms, hairpins, stem-and-loop structures and the like.

A further embodiment relates to a transformed organism such as an organism selected from plant cells, bacteria, yeast, baculovirus, protozoa, nematodes and algae. The transformed organism comprises a DNA molecule of the embodiments, an expression cassette comprising the DNA molecule or a vector comprising the expression cassette, which may be stably incorporated into the genome of the transformed organism.

The sequences of the embodiments are provided in DNA constructs for expression in the organism of interest. The construct will include 5′ and 3′ regulatory sequences operably linked to a sequence of the embodiments. The term “operably linked” as used herein refers to a functional linkage between a promoter and/or a regulatory sequence and a second sequence, wherein the promoter and/or regulatory sequence initiates, mediates, and/or affects transcription of the DNA sequence corresponding to the second sequence. Generally, operably linked means that the nucleic acid sequences being linked are contiguous and where necessary to join two protein coding regions in the same reading frame. The construct may additionally contain at least one additional gene to be cotransformed into the organism. Alternatively, the additional gene(s) can be provided on multiple DNA constructs.

Such a DNA construct is provided with a plurality of restriction sites for insertion of the polypeptide gene sequence of the disclosure to be under the transcriptional regulation of the regulatory regions. The DNA construct may additionally contain selectable marker genes.

The DNA construct will generally include in the 5′ to 3′ direction of transcription: a transcriptional and translational initiation region (i.e., a promoter), a DNA sequence of the embodiments, and a transcriptional and translational termination region (i.e., termination region) functional in the organism serving as a host. The transcriptional initiation region (i.e., the promoter) may be native, analogous, foreign or heterologous to the host organism and/or to the sequence of the embodiments. Additionally, the promoter or regulatory sequence may be the natural sequence or alternatively a synthetic sequence. The term “foreign” as used herein indicates that the promoter is not found in the native organism into which the promoter is introduced. As used herein, the term “heterologous” in reference to a sequence means a sequence that originates from a foreign species or, if from the same species, is substantially modified from its native form in composition and/or genomic locus by deliberate human intervention. As used herein, a chimeric gene comprises a coding sequence operably linked to a transcription initiation region that is heterologous to the coding sequence. Where the promoter is a native or natural sequence, the expression of the operably linked sequence is altered from the wild-type expression, which results in an alteration in phenotype.

In some embodiments the DNA construct comprises an S-gene polynucleotide encoding a clathrin adaptor medium subunit polypeptide, a pentatricopeptide repeat-containing polypeptide, or an RPS2-like resistance polypeptide of the embodiments. In some embodiments the DNA construct comprises an S-gene polynucleotide encoding a fusion protein comprising a polypeptide of the embodiments.

In some embodiments the DNA construct may also include a transcriptional enhancer sequence. As used herein, the term an “enhancer” refers to a DNA sequence which can stimulate promoter activity, and may be an innate element of the promoter or a heterologous element inserted to enhance the level or tissue-specificity of a promoter. Various enhancers include, for example, introns with gene expression enhancing properties in plants (US Patent Application Publication Number 2009/0144863, the ubiquitin intron (i.e., the maize ubiquitin intron 1 (see, for example, NCBI sequence S94464)), the omega enhancer or the omega prime enhancer (Gallie, et al, (1989) Molecular Biology of RNA ed. Cech (Liss, New York) 237-256 and Gallie, el al, (1987) Gene 60:217-25), the CaMV 35S enhancer (see, e.g., Benfey, et al, (1990) EMBO J. 9:1685-96) and the enhancers of U.S. Pat. No. 7,803,992 may also be used. The above list of transcriptional enhancers is not meant to be limiting. Any appropriate transcriptional enhancer can be used in the embodiments.

The termination region may be native with the transcriptional initiation region, may be native with the operably linked DNA sequence of interest, may be native with the plant host or may be derived from another source (i.e., foreign or heterologous to the promoter, the sequence of interest, the plant host or any combination thereof).

Convenient termination regions are available from the Ti-plasmid of A. tumefaciens, such as the octopine synthase and nopaline synthase termination regions. See also, Guerineau, et al, (1991) Mol Gen. Genet. 262:141-144; Proudfoot, (1991) Cell 64:671-674; Sanfacon, et al, (1991) Genes Dev. 5:141-149; Mogen, et al, (1990) Plant Cell 2:1261-1272; Munroe, et al, (1990) Gene 91:151-158; Balias, et al, (1989) Nucleic Acids Res. 17:7891-7903 and Joshi, et al, (1987) Nucleic Acid Res. 15:9627-9639.

Where appropriate, a nucleic acid may be optimized for increased expression in the host organism. Thus, where the host organism is a plant, the synthetic nucleic acids can be synthesized using plant-preferred codons for improved expression. See, for example, Campbell and Gowri, (1990) Plant Physiol. 92:1-11 for a discussion of host-preferred usage. For example, although nucleic acid sequences of the embodiments may be expressed in both monocotyledonous and dicotyledonous plant species, sequences can be modified to account for the specific preferences and GC content preferences of monocotyledons or dicotyledons as these preferences have been shown to differ (Murray et al. (1989) Nucleic Acids Res. 17:477-498). Thus, the plant-preferred for a particular amino acid may be derived from known gene sequences from plants.

Additional sequence modifications are known to enhance gene expression in a cellular host. These include elimination of sequences encoding spurious polyadenylation signals, exon-intron splice site signals, transposon-like repeats, and other well-characterized sequences that may be deleterious to gene expression. The GC content of the sequence may be adjusted to levels average for a given cellular host, as calculated by reference to known genes expressed in the host cell. The term “host cell” as used herein refers to a cell which contains a vector and supports the replication and/or expression of the expression vector is intended. Host cells may be prokaryotic cells such as E. coli or eukaryotic cells such as yeast, insect, amphibian or mammalian cells or monocotyledonous or dicotyledonous plant cells. An example of a dicotyledonous host cell is a spinach host cell. When possible, the sequence is modified to avoid predicted hairpin secondary mRNA structures.

In preparing the expression cassette, the various DNA fragments may be manipulated so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites or the like. For this purpose, in vitro mutagenesis, primer repair, restriction, annealing, resubstitutions, e.g., transitions and transversions, may be involved.

A number of promoters can be used in the practice of the embodiments. The promoters can be selected based on the desired outcome. The nucleic acids can be combined with constitutive, tissue-preferred, inducible or other promoters for expression in the host organism.

### Plant Transformation

The methods of the embodiments involve introducing a polypeptide or polynucleotide into a plant. “Introducing” is as used herein means presenting to the plant the polynucleotide or polypeptide in such a manner that the sequence gains access to the interior of a cell of the plant. The methods of the embodiments do not depend on a particular method for introducing a polynucleotide or polypeptide into a plant, only that the polynucleotide(s) or polypeptide(s) gains access to the interior of at least one cell of the plant. Methods for introducing polynucleotide(s) or polypeptide(s) into plants include, but are not limited to, stable transformation methods, transient transformation methods, and virus-mediated methods.

“Stable transformation” as used herein means that the nucleotide construct introduced into a plant integrates into the genome of the plant and is capable of being inherited by the progeny thereof. “Transient transformation” as used herein means that a polynucleotide is introduced into the plant and does not integrate into the genome of the plant or a polypeptide is introduced into a plant.

Transformation protocols as well as protocols for introducing nucleotide sequences into plants may vary depending on the type of plant or plant cell, i.e., monocot or dicot, targeted for transformation. Suitable methods of introducing nucleotide sequences into plant cells and subsequent insertion into the plant genome include microinjection (Crossway, et al., (1986) Biotechniques 4:320-334), electroporation (Riggs, et al., (1986) Proc. Natl. Acad. Sci. USA 83:5602-5606), Agrobacterium-mediated transformation (U.S. Pat. Nos. 5,563,055 and 5,981,840), direct gene transfer (Paszkowski, et al., (1984) EMBO J. 3:2717-2722) and ballistic particle acceleration (see, for example, U.S. Pat. Nos. 4,945,050; 5,879,918; 5,886,244 and 5,932,782; Tomes, et al., (1995) in Plant Cell, Tissue, and Organ Culture: Fundamental Methods, ed. Gamborg and Phillips, (Springer-Verlag, Berlin) and McCabe, et al., (1988) Biotechnology 6:923-926) and Led transformation (WO 00/28058). For potato transformation see, Tu, et al., (1998) Plant Molecular Biology 37:829-838 and Chong, et al., (2000) Transgenic Research 9:71-78. Additional transformation procedures can be found in Weissinger, et al., (1988) Ann. Rev. Genet. 22:421-477; Sanford, et al., (1987) Particulate Science and Technology 5:27-37 (onion); Christou, et al., (1988) Plant Physiol. 87:671-674 (soybean); McCabe, et al., (1988) Bio/Technology 6:923-926 (soybean); Finer and McMullen, (1991) In Vitro Cell Dev. Biol. 27P: 175-182 (soybean); Singh, et al., (1998) Theor. Appl. Genet. 96:319-324 (soybean); Datta, et al., (1990) Biotechnology 8:736-740 (rice); Klein, et al., (1988) Proc. Natl. Acad. Sci. USA 85:4305-4309 (maize); Klein, et al., (1988) Biotechnology 6:559-563 (maize); U.S. Pat. Nos. 5,240,855; 5,322,783 and 5,324,646; Klein, et al., (1988) Plant Physiol. 91:440-444 (maize); Fromm, et al., (1990) Biotechnology 8:833-839 (maize); Hooykaas-Van Slogteren, et al., (1984) Nature (London) 311:763-764; U.S. Pat. No. 5,736,369 (cereals); Bytebier, et al., (1987) Proc. Natl. Acad. Sci. USA 84:5345-5349 (Liliaceae); De Wet, et al., (1985) in The Experimental Manipulation of Ovule Tissues, ed. Chapman, et al., (Longman, New York), pp. 197-209 (pollen); Kaeppler, et al., (1990) Plant Cell Reports 9:415-418 and Kaeppler, et al., (1992) Theor. Appl. Genet. 84:560-566 (whisker-mediated transformation); D'Halluin, et al., (1992) Plant Cell 4:1495-1505 (electroporation); Li, et al., (1993) Plant Cell Reports 12:250-255 and Christou and Ford, (1995) Annals of Botany 75:407-413 (rice); Osjoda, et al., (1996) Nature Biotechnology 14:745-750 (maize via Agrobacterium tumefaciens).

Methods to Introduce Genome Editing Technologies into Plants

In some embodiments, polynucleotide compositions can be introduced into the genome of a plant using genome editing technologies, or endogenous polynucleotides in the genome of a plant may be edited using genome editing technologies. For example, the identified polynucleotides can be introduced into a desired location in the genome of a plant through the use of double-stranded break technologies such as TALENS, meganucleases, zinc finger nucleases, CRISPR-Cas, and the like.

In some embodiments, where an S-gene has been identified in a genome, genome editing technologies may be used to alter or modify the polynucleotide sequence. Site specific modifications that can be introduced into the desired gene allele polynucleotide include those produced using any method for introducing site specific modification, including, but not limited to, through the use of gene repair oligonucleotides (e.g. US Publication 2013/0019349), or through the use of double-stranded break technologies such as TALENs, meganucleases, zinc finger nucleases, CRISPR-Cas, and the like. Such technologies can be used to modify the polynucleotide through the insertion, deletion or substitution of nucleotides within the polynucleotide. Alternatively, double-stranded break technologies can be used to add additional nucleotide sequences to the polynucleotide. Additional sequences that may be added include, additional expression elements, such as enhancer and promoter sequences. In another embodiment, genome editing technologies may be used to position additional disease resistant proteins in close proximity to the S-gene polynucleotide within the genome of a plant, in order to generate molecular stacks of disease resistant proteins. An “altered target site,” “altered target sequence,” “modified target site,” and “modified target sequence” are used interchangeably herein and refer to a target sequence as disclosed herein that comprises at least one alteration when compared to non-altered target sequence. Such “alterations” include, for example: (i) replacement of at least one nucleotide (substitution, such as an GC to AT transition or such as a G to C or A to T transversion), (ii) a deletion of at least one nucleotide, (iii) an insertion of at least one nucleotide, or (iv) any combination of (i)-(iii).

### Embodiments

The following numbered embodiments also form part of the present disclosure:

1. A method of reducing susceptibility to a pathogen in a plant, the method comprising: introducing one or more nucleotide modifications at a genomic locus of the plant, wherein the genomic locus comprises a susceptibility gene (S-gene) encoding a clathrin adaptor medium subunit protein, a pentatricopeptide repeat-containing family protein, or an RPS2-like resistance protein.

2. The method of embodiment 1, wherein the one or more nucleotide modifications comprise a plurality of transitions such as GC to AT, transversions such as G to C or A to T, insertions, deletions, or a combination thereof in the S-gene.

3. The method of embodiment 1 or embodiment 2, wherein the one or more nucleotide modifications are introduced by a mutagen.

4. The method of embodiment 3, wherein the mutagen is ethyl methyl sulfonate (EMS).

5. The method of embodiment 1 or embodiment 2, wherein the one or more nucleotide modifications are introduced through a targeted DNA modification.

6. The method of embodiment 5, wherein the targeted DNA modification is introduced by an RNA-guided endonuclease.

7. The method of embodiment 1 or embodiment 2, wherein the one or more nucleotide modifications are introduced by introgression.

8. The method of any one of embodiments 1-7, wherein the one or more nucleotide modifications result in altered expression or activity of a protein encoded by the S-gene.

9. The method of any one of embodiments 1-8, wherein the one or more nucleotide modifications comprises an insertion, a deletion, or a single nucleotide polymorphism (SNP).

10. The method of any one of embodiments 1-9, wherein the one or more nucleotide modifications comprise at least one non-synonymous single nucleotide polymorphism (nsSNP) in the S-gene coding sequence.

11. The method of any one of embodiments 1-10, wherein the one or more nucleotide modifications are present within (a) the coding region, (b) non-coding region, (c) regulatory sequence, or (d) untranslated region of the S-gene.

12. The method of any one of embodiments 1-11, wherein the one or more nucleotide modifications results in one or more of the following: (a) altered expression of the S-gene; (b) generation of one or more alternative spliced transcripts of the S-gene; (c) deletion or alteration of one or more DNA binding domains; (d) frameshift mutation in one or more exons of the S-gene; (e) deletion or alteration of a substantial portion of the S-gene, or deletion of the full-length open reading frame of the S-gene; (f) repression or induction of an enhancer motif present within a regulatory region encoding the S-gene; (g) modification or deletion of one or more nucleotides of a regulatory element operably linked to the expression of the S-gene, wherein the regulatory element is present within a promoter, intron, 3′UTR, terminator, or a combination thereof.

13. The method of any one of embodiments 1-12, wherein the one or more nucleotide modifications result in a sequence selected from SEQ ID NOs: 1, 2, 93, 94, 169, or 170.

14. A method of reducing susceptibility to a pathogen in a plant, the method comprising: altering expression or activity of a protein encoded by a susceptibility gene (S-gene), wherein the protein is a clathrin adaptor medium subunit protein, a pentatricopeptide repeat-containing family protein, or an RPS2-like resistance protein.

15. The method of embodiment 14, wherein the altering comprises one or more mechanisms selected from RNA interference, genome editing including gene replacement or allele replacement, gene knockout, and gene knockdown.

16. The method of any one of embodiments 1-15, wherein the S-gene encodes a clathrin adaptor medium subunit protein comprising an amino acid sequence that is at least 80% identical to a sequence selected from SEQ ID NOs: 6, 64-92, 294-315.

17. The method of embodiment 16, wherein the S-gene encodes a clathrin adaptor medium subunit protein comprising an amino acid sequence that is at least 80% identical to the sequence of SEQ ID NO: 6.

18. The method of any one of embodiments 1-15, wherein the S-gene encodes a pentatricopeptide repeat-containing protein comprising an amino acid sequence that is at least 80% identical to a sequence selected from SEQ ID NOs: 98, 145-168, 326-330.

19. The method of embodiment 18, wherein the S-gene encodes a pentatricopeptide repeat-containing protein comprising an amino acid sequence that is at least 80% identical to the sequence of SEQ ID NO: 98.

20. The method of any one of embodiments 1-15, wherein the S-gene encodes an RPS2-like resistance protein comprising an amino acid sequence that is at least 80% identical to a sequence selected from SEQ ID NOs: 174, 219-240, 333.

21. The method of embodiment 20, wherein the S-gene encodes an RPS2-like resistance protein comprising an amino acid sequence that is at least 80% identical to the sequence of SEQ ID NO: 174.

22. The method of any one of embodiments 1-21, wherein the genomic locus comprises and is flanked by SEQ ID NO: 241 and SEQ ID NO: 251.

23. The method of any one of embodiments 1-22, wherein the plant is of the species Spinacia oleracea, and the pathogen is of the phylum Oomycota.

24. The method of embodiment 23, wherein the pathogen is at least one of the species selected from Peronospora farinosa (downy mildew); Peronospora effusa (downy mildew); Stemphylium botryosum f. sp. spinacia (Stemphylium leaf spot); Cladosporium variabile (leaf spot); Albugo occidentalis (white rust); Colletotrichum dematium f. sp. spinaciae (anthracnose); Phythium spp. (damping off/seedling blight); Fusarium oxysporum f. sp. spinaciae (fusarium wilt); Aphanomyces cochlioides (black root rot); and Verticillium dahlia (verticillium wilt).

25. The method of any one of embodiments 1-22, wherein the plant is of the genus Helianthus, and the pathogen is of the species Plasmopara halstedii (downy mildew).

26. The method of any one of embodiments 1-22, wherein the plant is of the species Zea mays, and the pathogen is of the species Peronosclerospora maydis (downy mildew).

27. The method of any one of embodiments 1-22, wherein the plant is of the species Beta vulgaris, and the pathogen is of the species Peronospora farinosa (downy mildew).

28. The method of any one of embodiments 1-22, wherein the plant is of the species Lactuca sativa, and the pathogen is of the species Bremia lactucae (downy mildew).

29. The method of any one of embodiments 1-22, wherein the plant is of the family Cucurbitaceae, and the pathogen is of the species Pseudoperonospora cubensis (downy mildew).

30. The method of any one of embodiments 1-22, wherein the plant is of the family Brassicaceae, and the pathogen is of the species Peronospora parasitica (downy mildew).

31. The method of any one of embodiments 1-22, wherein the plant is of the family Solanaceae, and the pathogen is of the species Peronospora hyoscyami (downy mildew).

32. A plant with reduced susceptibility to a pathogen comprising one or more nucleotide modifications at a genomic locus of the plant, wherein the genomic locus comprises a susceptibility gene (S-gene) encoding a clathrin adaptor medium subunit protein, a pentatricopeptide repeat-containing family protein, or an RPS2-like resistance protein.

33. The plant of embodiment 32, wherein the one or more nucleotide modifications comprise a plurality of transitions such as GC to AT, transversions such as G to C or A to T, insertions, deletions, or a combination thereof in the S-gene.

34. The plant of embodiment 32 or embodiment 33, wherein the one or more nucleotide modifications result in altered expression or activity of a protein encoded by the S-gene.

35. The plant of any one of embodiments 32-34, wherein the one or more nucleotide modifications comprise an insertion, a deletion, or a single nucleotide polymorphism (SNP).

36. The plant of any one of embodiments 32-35, wherein the one or more nucleotide modifications are present within (a) the coding region, (b) non-coding region, (c) regulatory sequence, or (d) untranslated region of the S-gene.

37. The plant of any one of embodiments 32-36, wherein the one or more nucleotide modifications comprise at least one non-synonymous single nucleotide polymorphism (nsSNP) in the S-gene coding sequence.

38. The plant of any one of embodiments 32-37, wherein the one or more nucleotide modifications results in one or more of the following: (a) altered expression of the S-gene; (b) generation of one or more alternative spliced transcripts of the S-gene; (c) deletion or alteration of one or more DNA binding domains; (d) frameshift mutation in one or more exons of the S-gene; (e) alteration or deletion of a substantial portion of the S-gene or deletion of the full-length open reading frame of the S-gene; (f) repression or induction of an enhancer motif present within a regulatory region encoding the S-gene; (g) modification or deletion of one or more nucleotides of a regulatory element operably linked to the expression of the S-gene, wherein the regulatory element is present within a promoter, intron, 3′UTR, terminator, or a combination thereof.

39. The plant of any one of embodiments 32-38, wherein the one or more nucleotide modifications result in a sequence selected from SEQ ID NOs: 1, 2, 93, 94, 169, or 170.

40. A plant with reduced susceptibility to a pathogen comprising altered expression or activity of a protein encoded by a susceptibility gene (S-gene), wherein the protein is a clathrin adaptor medium subunit protein, a pentatricopeptide repeat-containing family protein, or an RPS2-like resistance protein.

41. The plant of any one of embodiments 32-40, wherein the S-gene encodes a clathrin adaptor medium subunit protein comprising an amino acid sequence that is at least 80% identical to a sequence selected from SEQ ID NOs: 6, 64-92, 294-315.

42. The plant of any one of embodiments 32-40, wherein the S-gene encodes a pentatricopeptide repeat-containing protein comprising an amino acid sequence that is at least 80% identical to a sequence selected from SEQ ID NOs: 98, 145-168, 326-330.

43. The plant of any one of embodiments 32-40, wherein the S-gene encodes an RPS2-like resistance protein comprising an amino acid sequence that is at least 80% identical to a sequence selected from SEQ ID NOs: 174, 219-240, 333.

44. The plant of any one of embodiments 32-43, wherein the genomic locus comprises and is flanked by SEQ ID NO: 241 and SEQ ID NO: 251.

45. The plant of any one of embodiments 32-44, wherein the plant is of the species Spinacia oleracea, and the pathogen is of the phylum Oomycota.

46. The plant of embodiment 45, wherein the pathogen is at least one of the species selected from Peronospora farinosa (downy mildew); Peronospora effusa (downy mildew); Stemphylium botryosum f. sp. spinacia (Stemphylium leaf spot); Cladosporium variabile (leaf spot); Albugo occidentalis (white rust); Colletotrichum dematium f. sp. spinaciae (anthracnose); Phythium spp. (damping off/seedling blight); Fusarium oxysporum f. sp. spinaciae (fusarium wilt); Aphanomyces cochlioides (black root rot); and Verticillium dahlia (verticillium wilt).

47. The plant of any one of embodiments 32-44, wherein the plant is of the genus Helianthus, and the pathogen is of the species Plasmopara halstedii (downy mildew).

48. The plant of any one of embodiments 32-44, wherein the plant is of the species Zea mays, and the pathogen is of the species Peronosclerospora maydis (downy mildew).

49. The plant of any one of embodiments 32-44, wherein the plant is of the species Beta vulgaris, and the pathogen is of the species Peronospora farinosa (downy mildew).

50. The plant of any one of embodiments 32-44, wherein the plant is of the species Lactuca sativa, and the pathogen is of the species Bremia lactucae (downy mildew).

51. The plant of any one of embodiments 32-44, wherein the plant is of the family Cucurbitaceae, and the pathogen is of the species Pseudoperonospora cubensis (downy mildew).

52. The plant of any one of embodiments 32-44, wherein the plant is of the family Brassicaceae, and the pathogen is of the species Peronospora parasitica (downy mildew).

53. The plant of any one of embodiments 32-44, wherein the plant is of the family Solanaceae, and the pathogen is of the species Peronospora hyoscyami (downy mildew).

54. A method of identifying a spinach plant with increased resistance to downy mildew, the method comprising: a) detecting in a spinach plant an allele associated with increased resistance to downy mildew, wherein the resistant allele comprises a “T” at Spov3_chr4_93275081, an “A” at Spov3_chr4_105049870, an “A” at Spov3_chr4_107241402, an “A” at Spov3_chr4_109546568, an “A” at Spov3_chr4_109698808, an “A” at Spov3_chr4_112008390, a “T” at Spov3_chr4_112574123, a “T” at Spov3_chr4_117783935, an “A” at Spov3_chr4_118191085, an “A” at Spov3_chr4_118788268, or an “A” at Spov3_chr4_121142541; and b) identifying a spinach plant comprising the resistant gene allele as having increased resistance to downy mildew.

55. A method of identifying and/or selecting a spinach plant having increased resistance to downy mildew, the method comprising: a) screening a population with a marker located within an interval on chromosome 4 comprising and flanked by SEQ ID NO: 241 and SEQ ID NO: 251 to determine if one or more plants from the population comprise a resistant allele; and b) selecting from said population at least one plant comprising the resistant allele.

56. The method of embodiment 55, further comprising: c) crossing the plant of b) to a second plant; and d) obtaining a progeny plant that has the resistant allele.

57. The method of embodiment 55 or embodiment 56, wherein the resistant allele comprises a “T” at Spov3_chr4_93275081, an “A” at Spov3_chr4_105049870, an “A” at Spov3_chr4_107241402, an “A” at Spov3_chr4_109546568, an “A” at Spov3_chr4_109698808, an “A” at Spov3_chr4_112008390, a “T” at Spov3_chr4_112574123, a “T” at Spov3_chr4_117783935, an “A” at Spov3_chr4_118191085, an “A” at Spov3_chr4_118788268, or an “A” at Spov3_chr4_121142541.

58. A spinach plant comprising broad-spectrum resistance against Peronospora effusa, wherein the resistance is conferred by an introgression fragment comprising a susceptibility gene (S-gene).

59. The spinach plant of embodiment 58, wherein the S-gene is present in plants grown from seeds of Nr.22-18, a representative sample of seeds having been deposited under accession number NCIMB 43773.

60. The spinach plant of embodiment 58 or embodiment 59, wherein the introgression fragment comprises one or more of SEQ ID NOs: 1, 93, 169.

61. The spinach plant of any one of embodiments 58-60, wherein the spinach plant comprises at least one allele selected from a “T” at Spov3_chr4_93275081, an “A” at Spov3_chr4_105049870, an “A” at Spov3_chr4_107241402, an “A” at Spov3_chr4_109546568, an “A” at Spov3_chr4_109698808, an “A” at Spov3_chr4_112008390, a “T” at Spov3_chr4_112574123, a “T” at Spov3_chr4_117783935, an “A” at Spov3_chr4_118191085, an “A” at Spov3_chr4_118788268, and an “A” at Spov3_chr4_121142541.

62. The spinach plant of any one of embodiments 58-61, wherein the spinach plant comprises resistance at least against Peronospora effusa races 1-20 including but not limited to Pe:4+, Pe:5, Pe:12, Pe:14, Pe:16, Pe:17 and Pe:19, or at least against Peronospora effusa races 1-19 including but not limited to Pe:4+, Pe:5, Pe:12, Pe:14, Pe:16, Pe:17 and Pe:19 and uncategorized Pe-isolates 4US and UA2016-21A (Pe:21A).

63. The spinach plant of any one of embodiments 58-62, wherein the spinach plant is a hybrid plant.

64. The spinach plant of any one of embodiments 58-62, wherein spinach plant is an inbred plant.

65. A seed from which a spinach plant of any one of embodiments 58-64 can be grown.

66. A leaf of a spinach plant of any one of embodiments 58-64.

67. A progeny plant of a spinach plant of any one of embodiments 58-64.

68. A part of the spinach plant of any one of embodiments 58-64, wherein the part is selected from a stem, a cutting, a petiole, a cotyledon, a flower, an anther, a pollen, an ovary, a root, a root tip, a protoplast, a callus, a microspore, a stalk, an ovule, a shoot, a seed, an embryo, an embryo sac, a cell, a meristem, a bud, or a leaf.

69. A cell culture or tissue culture comprising a cell or a tissue derived from the part of embodiment 68.

70. A food product comprising harvested leaves of the spinach plant of any one of embodiments 58-64.

71. A container comprising the spinach plant of any one of embodiments 58-64, in a growth substrate for harvest of leaves from the plant.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

The following examples are offered by way of illustration and not by way of limitation.

### EXAMPLES

Note: Although the following examples 1 to 7 mainly focus on and show the results of experiments performed with P. effusa race Pe:16, very similar results were obtained with P. effusa races Pe:4+, Pe:5, Pe:12, Pe:14, Pe:17, Pe:19 and Pe:21A.

### Example 1: Identification of EMS Induced Variation Conferring Resistance to P. effusa

Spinach inbred line B11-5Sep. 7, 1941-2sel. (hereafter referred to as B11-509 line) is resistant to races Pe:1 to Pe:15, Pe:17, and Pe:18 but is susceptible to Pe:16, Pe:19 and some other, uncategorized, Pe-isolates such as 4US and UA2016-21A. Seeds of the B11-509 line were treated with 0.4% of the mutagen EthylMethaneSulphonate (EMS) to induce genome-wide GC>AT transitions. EMS can also induce other changes at a low rate. Individual M1 plants were selfed and seedlings of M2 families were used for screening for DM resistance by inoculation with isolate Pe:16. An M2 family, EMS22, was found to segregate for DM-resistant and susceptible plants. M3 families were obtained by selfing three resistant and seven susceptible M2 plants. Retesting with isolate Pe:16 confirmed the resistance and susceptibility pattern in these ten M3 families. Testing with uncategorized isolate Pe:21A showed that the resistance is working more broadly, and is likely effective against all other current and future races of Peronospora effusa due to the mechanism of action of this resistance.

Two approaches were taken to investigate which chromosomal region, and ultimately which specific mutation is causal for the observed DM resistance: Bulk Segregant Analysis (BSA) of selfed M3 families and genetic mapping in Viroflay backcross F2 populations.

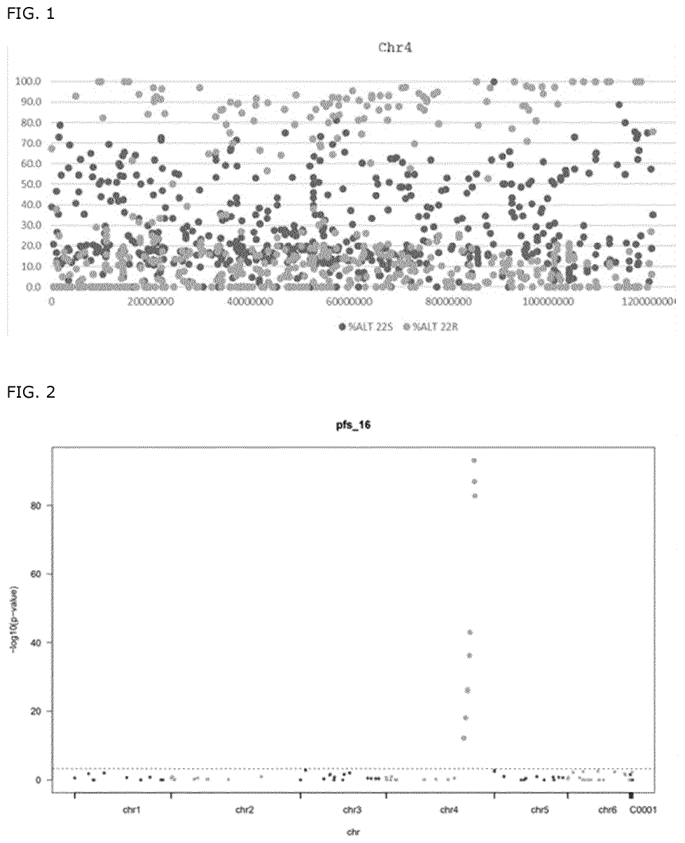

The BSA was performed on pooled gDNA of three resistant and seven susceptible M3 families (R and S-pool). Resequencing of the two pools and mapping to the Viroflay reference genome Phytozome: Soleracea_575_Spov3 yielded variants of the (1) B11-509 line used for EMS treatment (2) the R-pool and (3) S-pool. Scanner plots of SNP coverage of the two pools for six chromosomes showed that the highest percentage of near homozygous R-pool SNPs combined with very low percentage S-pool SNPs and absolute absence in the B11-509 data, was detected in a 20 Mb proximal and a 6.2 Mb distal region of Chr4 (FIG. 1). SNPs in these regions were validated for their effect on gene models of the reference genome: several SNPs were in exons of predicted genes and three SNPs cause non-synonymous amino acid changes. Said three non-synonymous changes are predicted to have an effect on the protein of gene Spov3_chr4.04649, a putative resistance RPS2-like protein, on gene Spov3_chr4.04219, relating to a putative Pentatricopeptide repeat-containing family protein and on Spov3_chr4.04752, relating to a putative Clathrin adaptor medium subunit protein.

Note: gene model Spov3_chr4.04129 is not correct and was replaced by Sp75 gene model Spo10764 (SEQ ID NO: 96, see Table 3).

For genetic mapping, backcrosses of three resistant M3 plants to Viroflay (susceptible to all races of Pe) were made and segregating F2-plants for each cross were phenotyped with Pe:16 and genotyped in KASP assays with 142 R-pool specific SNPs from the BSA, covering all six chromosomes. Some two thousand F2 plants of eleven F2 offspring were phenotyped and showed a significantly 1R: 3S ratio as expected for recessive impaired S-gene inheritance. The KASP genotyping yielded 91 informative, segregating SNPs and all positive and negative control scores were consistent. GWAS analysis of 91 SNPs informative for EMS transitions and susceptibility/resistance scores to Pe:16 showed that as in the BSA, a clear correlation of 11 SNPs at the distal end of Chr4 was detected (FIG. 2). This includes the three BSA-derived SNPs in the three candidate S-genes.

### Example 2: Identification of the Causal Gene for Resistance (S-Gene)

Note: in the present context, the S-gene or susceptibility gene, may also be denoted as the causal gene for resistance. ‘Resistance’ in the present context is the loss of susceptibility.

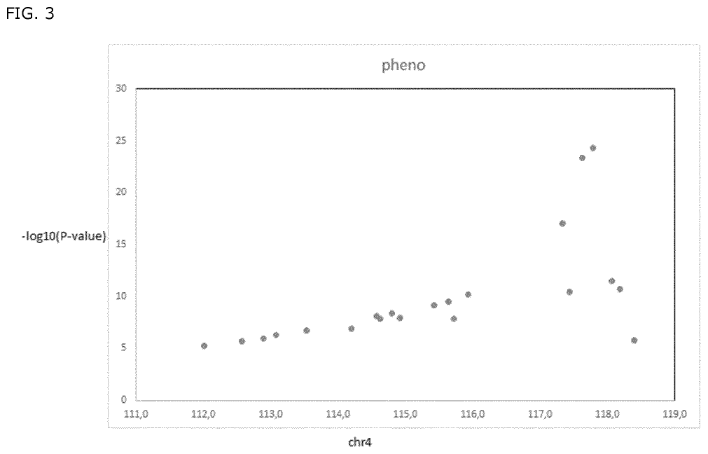

A clear correlation of the susceptibility-locus (S-locus) with the mentioned 11 SNPs at the distal end of Chr4 was detected (see Example 1, and FIG. 2). This region spans 6.2 Mb. For further fine-mapping of the S-locus and to address the stability of the S-locus in other spinach backgrounds, two sets of new crosses were made; crossings of a specific EMS22 inbred line (X20-10-1) with Viroflay and crossings of EMS22 inbred lines to three PV-elite lines containing the DM-resistance locus referred to as RPF13 (X17-003). Segregating F2-plants for each cross were phenotyped with Pe:16 and genotyped in KASP assays with the 11 SNPs. Some thousand F3 plants of both backgrounds were phenotyped and showed a significantly 1R:3S ratio as expected for recessive impaired S-gene inheritance. No notable phenotypic differences were detected within the two backgrounds. The genotypic results showed that the underlying mutation leading to resistance to Pe:16 was mapped proximal of SNP 117783935_GA (SEQ ID NO: 337) in the candidate gene Spov3_chr4.04649, a putative resistance RPS2-like protein. For additional fine-mapping, 70 SNPs specific for B11-509/Viroflay were selected in the genomic region 112-118 Mb and used to detect recombination in the two backgrounds. The highest correlation was found for three SNPs in the region of the RPS2-like candidate gene (FIG. 3). In one population of the two backgrounds, recombination events closest to the RPS2-like gene were detected resulting in a region of 0.62 Mb between two flanking SNPs, SNP 117443435_AG (SEQ ID NO: 336) and >118064634_TC (SEQ ID NO: 338) (sequences listed below). The subsequent research focused on said RPS2-like gene.

SNP Listing.

>117443435_AG-SEQ ID NO: 336
AAGGAACTCTTGAAAGATACAATGGTGAAGAAGCAGAGCTTAATAAAGACTAAAATATGATTA
AGTTAGTGAAAGAAAAGGATTCAATGGCCCTGCATGTAGCAAACAAATAGTGTCACAAAAGTG
AAAAAGATGTACACCGCATAATCCACAACTAATGATGCAAGCTTTAGTTGAGGTTCAAGGCTA
AAACAATCACCAT
>117783935_GA-SEQ ID NO: 337
TGTAAGGTCTCAGCATTGGTGCATATTAGAGAGCTATCATCTACAGGTTGGTTTTGAGGTGAG
ATCTGAGCAACCATACAACAGAAGAGTATCACTCTCCGAGATGTTCTTTGACTGGTCAAGAGG
GAGACTACGTTGTGCTTCCAAGTAACATCCAGGAGTTGTATGTTGATAATTGCAGAGACTTTC
GTTGCCTATCTGA
>118064634_TC-SEQ ID NO: 338
TTTTTGGTCCCTACAAGATTATTTTCTGCCAAACTCGACTCTACCCATTCAATACTTCGTATGAA
AAGAGGCTTGAACATAGTCTCTGTCATCTTCATCGTCCAAAGCAACCACCGCACCAATGACAT
GTTTCTCCACGAAAGTAATATTCTCAATTGATTCAAGGTGCTGACTGCGGAGATCCAGAGCCA
GCATACAGAAG

### Example 3: Identification of Causal Mutations for Plant Resistance Against P. effusa

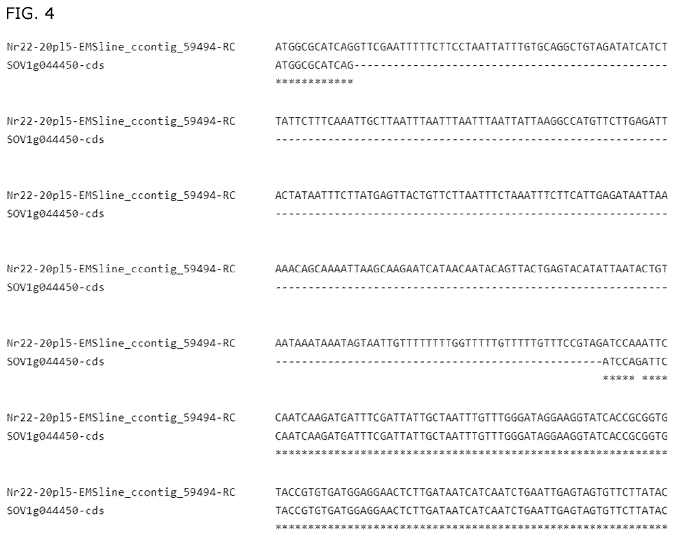

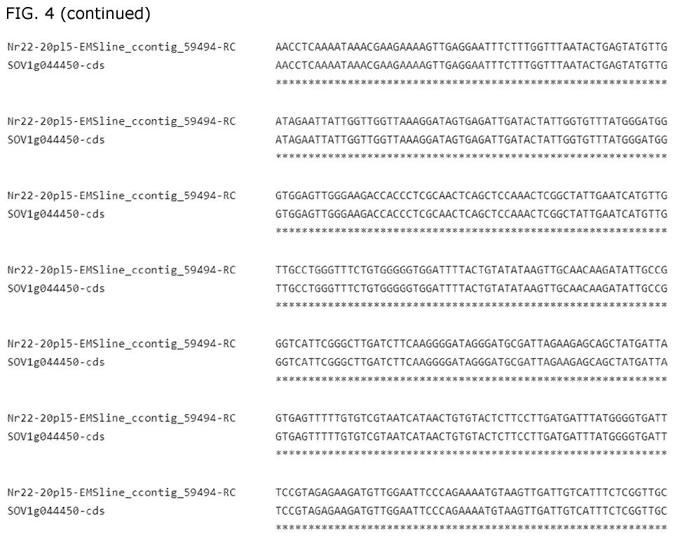

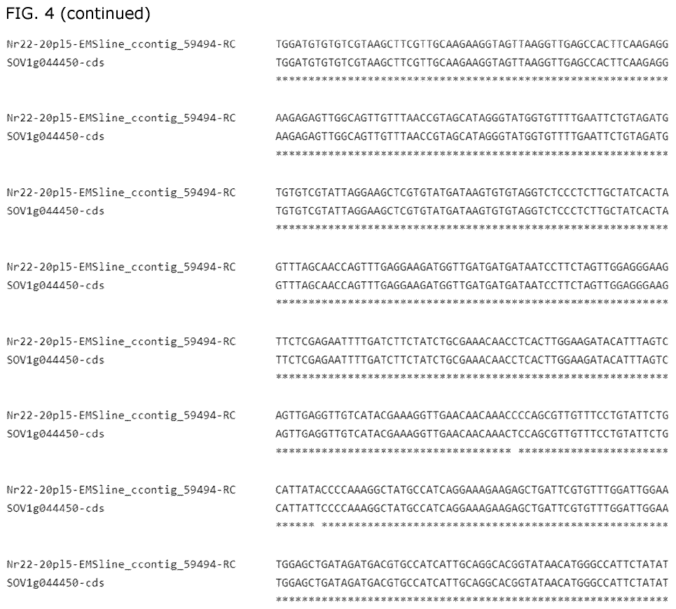

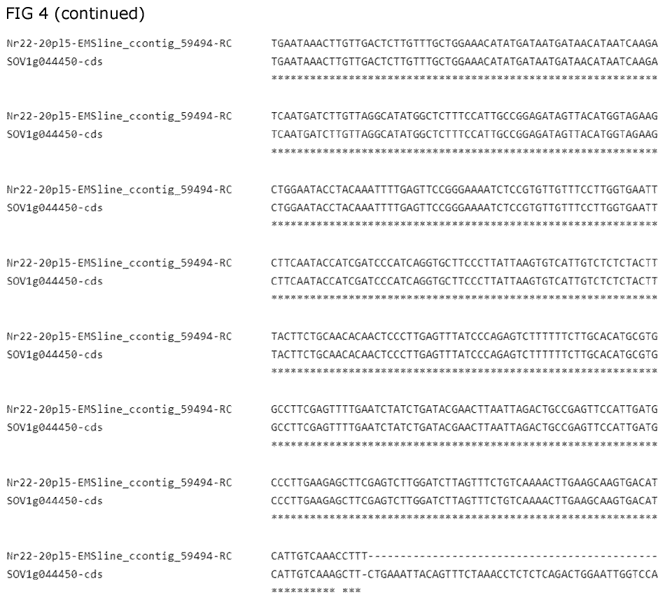

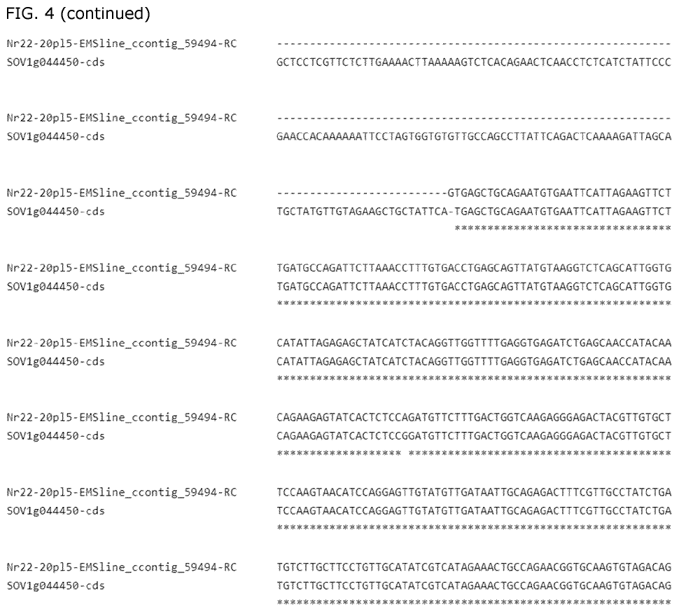

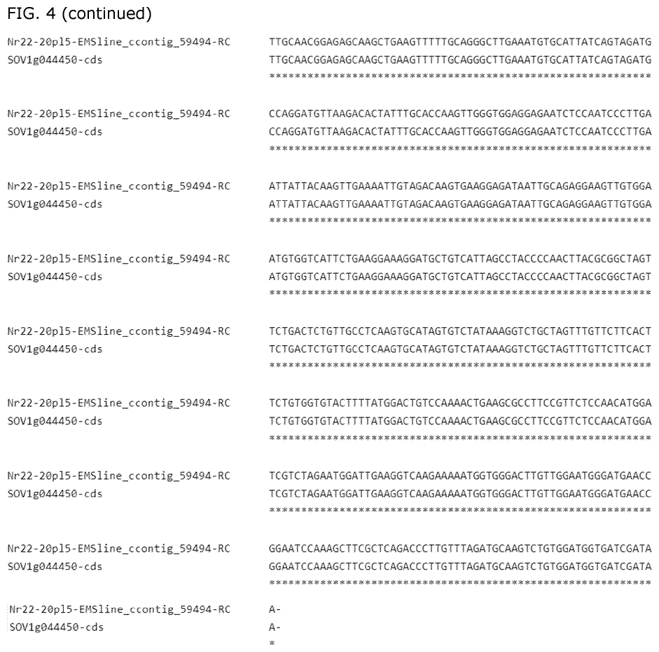

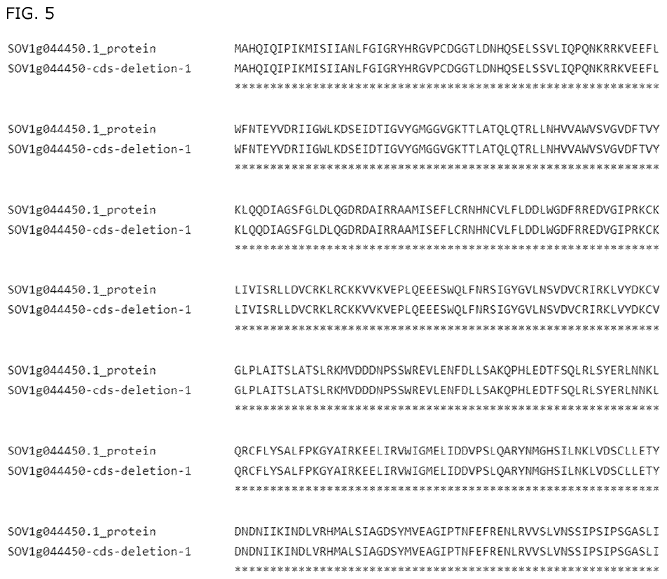

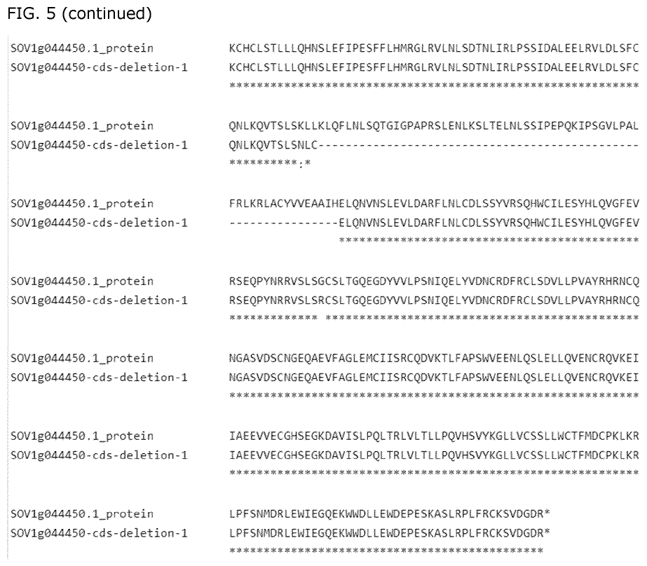

In addition to the reference genome Viroflay SpoV3 used in the aforementioned BSA, new high-quality references were made of both Viroflay and inbred line B11-509. The Chr4 distal region of 0.62 Mb containing the S-locus was compared by Whole Genome Alignment analysis. Short read sequences of one individual mutant M3-plant (EMS22-20) were obtained and used for mapping to the new references. It is known that next to genome-wide GC>AT transitions, EMS can also induce other changes at a low rate. Therefore, the new read mappings were used in both Small Nucleotide Variation calling as well visual inspection of larger deletions and duplications. One larger deletion was detected in Gene Model SOV1g044450, ortholog of candidate gene Spov3_chr4.04649 (RPS2-like) in the new high-quality Viroflay reference. The deletion is close to the previously detected G>A transition causing the neutral non-synonymous amino acid change. The deletion is 191 bp and was confirmed in read mappings of a pool of resistant plants used in the BSA. The deletion was absent in read mappings of both B11-509 and a pool of susceptible plants used in the BSA. The deletion could be reconstructed by re-assembling unmapped reads of EMS22-20 in contig_59494. Three additional changes were detected in an alignment of contig_59494 and SOV1g044450: one rare G>C transition 4 bp of the left border of the deletion, one T insertion at the left border, and one G insertion at the right border (FIG. 4, and sequences listed below). All changes to SOV19044450 were confirmed by sequencing of individual PCR products of resistant EMS-induced plants and were absent in PCR products of susceptible families and controls. Furthermore, a full-length cDNA of EMS22-20 and B11-509 was cloned and sequenced confirming the changes. Due to the insertions the deletion is 189 bp and causes one amino acid change, K491N. The G>C transition also causes an amino acid change L493C. The third amino acid change G416R is caused by the G>A transition detected in the BSA (FIG. 5, and sequences listed below). The RPS2-like gene encodes for a putative resistance gene of the NLR class containing an N-terminal Nucleotide Binding motif and five Leucine Rich Repeat (LRR) motifs. The 189 bp deletion causes the removal of 2 C-terminal LRR motifs in the protein. In summary, EMS-treated mutants of family EMS22 that are resistant to Peronospora effusa have causal mutations in gene SOV19044450: two nucleotide transitions, two nucleotide insertions and one 189 bp deletion causing three amino acid changes and deleting 63 amino acids comprising two C-terminal LRRs.

Nr22-20pI5-EMSline_S3_L001_(paired)_contig_59494-RC SOV1g044450.1 genomic
sequence/locus-
SEQ ID NO: 339
TATATATAACAATGACAATACTTATATAACCGATGACAATATATAACAAGTTAGCAACACTATTT
AAGTGTGGGCCATCTTTTCAACTTTTTTTTTTTTTTTAAGGGTGGGTATGGATTTTTTTTGTCGC
TGGTGGACAGCGATGTAGCCAACCTCTAATACGGAGTATGATGAAAGATATAAGTTTGAGCAA
AAAAAGTCTTACACGCATATAATAAATTTATTGCATGTCAGGAAAACTACTTCCTAAATTTTTGT
AACTTTTAACTAATTTTTTGTAACTTTTATATTATAAAAAAAATTGATAGATAAACATGTGTTAAA
TGATTAATTTTTTACATTATTAACAAATTTGAAAAAATATTTTTTATTAAAATGAAAATTTTTATC
ACTAAAAAAAAAATACTGGAGTAGATGTCATTTACAAAGAGTAACTTTTAAGCATTTTAAAACA
ACTTTTACTCCAACTTATAATATTAAGTCTGAAATGGGTTGTATAATTATTAACATAGATTCCAT
AATTTTACTCTTTTTACCAAAAATTGCACTTAAGTTCATTTTCATTTCAATAGTTTTGTTACCGGC
TATCTTACGGATGTTTGTGTGACAAAGACTTTATTCTCATCACTTAATTTGGTCTGATATGATAA
GATCTGATCTGAAAGATGTATATGACTGCTTTTTGATTTTTTGTCCTTACGGATGTTTGTAAAAC
CAAGACTTTGTTCTCCTCATTTTTTGTCTAATATGATCTGATCTGAAAGTTATATAAGTGCTTTT
TTTATTTTTGTCTTTTCTAATTTGATATAATTTTTTTTCTGATCTGATCTAATTTTTCTGAACAATT
TCGCATTCATACAGACGTACTCCCTCCGTATTTATTTAAAGGATGCACTTGACTTTTCCGGCCG
TATTTATTTAAGAGATACACTTGCCATTTTTAGTAACTTATCAACCCCACCATCTAATTAAATAA
TACTCCCTCCATTCCTAAATAAGTGTCCAGTTCCCATAAATAGAGTTCCCTTATAAGTGTCCAG
TTTTCATTTTTGGACATACATCTTTCTCACTTTTCTATACACTTTTCTCACTTTTTCACACACTTTT
CCCATTTTTCTATAAATCATTATTACTTTTTATTACACATTCTACACTCTTCATACTTTTCAATCA
CCACATCCACTTTTATTTATACTTTATCAATAAACAATTATCTACTTTTTCCTTTCCCAAAAAAAA
AATATTCTCAATCATTACCTCTTACACACAATTCAGTTTTATTAATCACGCGTAAATGTCCAAAG
TGGACACTTATTTAGGAACGAAGGGAGTACATCTAATTTACCCTATGACCTACCATCCTATTAA
ACAAATAATTTCATAAACCCACCCCACCTCCCACCCCACCAAAATGACATGGTCCCACTTGTTT
ACTTATTAAAATATCACCCAATCCCACTTATTTTATTATTATATTTCATTTAATTTTTTTCTTAATA
CCCGTGCTCGACTAAGTGTATCCGTTAAATAAATACAAAGAGAGTAGTTAACAGGTTCACATTT
TTTAAAGTCTAGTCCGTTGGGCGATAGTAGACTTTTTTTGGGTTTAAATTAGTACCCAGTTTTC
TTAAATATGAGGAGCCTAGACATTTTAAGGAGTTAACAAAAATGGAGAGCACTTTTCTTCACTG
ATACAGAGTAGCTCATATTCGTCAGTTATGGCGCATCAGGTTCGAATTTTTCTTCCTAATTATTT
GTGCAGGCTGTAGATATCATCTTATTCTTTCAAATTGCTTAATTTAATTTAATTTAATTATTAAG
GCCATGTTCTTGAGATTACTATAATTTCTTATGAGTTACTGTTCTTAATTTCTAAATTTCTTCATT
GAGATAATTAAAAACAGCAAAATTAAGCAAGAATCATAACAATACAGTTACTGAGTACATATTA
ATACTGTAATAAATAAATAGTAATTGTTTTTTTTGGTTTTTGTTTTTGTTTCCGTAGATCCAAATT
CCAATCAAGATGATTTCGATTATTGCTAATTTGTTTGGGATAGGAAGGTATCACCGCGGTGTA
CCGTGTGATGGAGGAACTCTTGATAATCATCAATCTGAATTGAGTAGTGTTCTTATACAACCTC
AAAATAAACGAAGAAAAGTTGAGGAATTTCTTTGGTTTAATACTGAGTATGTTGATAGAATTAT
TGGTTGGTTAAAGGATAGTGAGATTGATACTATTGGTGTTTATGGGATGGGTGGAGTTGGGAA
GACCACCCTCGCAACTCAGCTCCAAACTCGGCTATTGAATCATGTTGTTGCCTGGGTTTCTGT
GGGGGTGGATTTTACTGTATATAAGTTGCAACAAGATATTGCCGGGTCATTCGGGCTTGATCT
TCAAGGGGATAGGGATGCGATTAGAAGAGCAGCTATGATTAGTGAGTTTTTGTGTCGTAATCA
TAACTGTGTACTCTTCCTTGATGATTTATGGGGTGATTTCCGTAGAGAAGATGTTGGAATTCCC
AGAAAATGTAAGTTGATTGTCATTTCTCGGTTGCTGGATGTGTGTCGTAAGCTTCGTTGCAAG
AAGGTAGTTAAGGTTGAGCCACTTCAAGAGGAAGAGAGTTGGCAGTTGTTTAACCGTAGCATA
GGGTATGGTGTTTTGAATTCTGTAGATGTGTGTCGTATTAGGAAGCTCGTGTATGATAAGTGT
GTAGGTCTCCCTCTTGCTATCACTAGTTTAGCAACCAGTTTGAGGAAGATGGTTGATGATGAT
AATCCTTCTAGTTGGAGGGAAGTTCTCGAGAATTTTGATCTTCTATCTGCGAAACAACCTCACT
TGGAAGATACATTTAGTCAGTTGAGGTTGTCATACGAAAGGTTGAACAACAAACCCCAGCGTT
GTTTCCTGTATTCTGCATTATACCCCAAAGGCTATGCCATCAGGAAAGAAGAGCTGATTCGTG
TTTGGATTGGAATGGAGCTGATAGATGACGTGCCATCATTGCAGGCACGGTATAACATGGGC
CATTCTATATTGAATAAACTTGTTGACTCTTGTTTGCTGGAAACATATGATAATGATAACATAAT
CAAGATCAATGATCTTGTTAGGCATATGGCTCTTTCCATTGCCGGAGATAGTTACATGGTAGA
AGCTGGAATACCTACAAATTTTGAGTTCCGGGAAAATCTCCGTGTTGTTTCCTTGGTGAATTCT
TCAATACCATCGATCCCATCAGGTGCTTCCCTTATTAAGTGTCATTGTCTCTCTACTTTACTTCT
GCAACACAACTCCCTTGAGTTTATCCCAGAGTCTTTTTTCTTGCACATGCGTGGCCTTCGAGTT
TTGAATCTATCTGATACGAACTTAATTAGACTGCCGAGTTCCATTGATGCCCTTGAAGAGCTTC
GAGTCTTGGATCTTAGTTTCTGTCAAAACTTGAAGCAAGTGACATCATTGTCAAACCTTT┌ ┐GT
GAGCTGCAGAATGTGAATTCATTAGAAGTTCTTGATGCCAGATTCTTAAACCTTTGTGACCTGA
GCAGTTATGTAAGGTCTCAGCATTGGTGCATATTAGAGAGCTATCATCTACAGGTTGGTTTTG
AGGTGAGATCTGAGCAACCATACAACAGAAGAGTATCACTCTCCAGATGTTCTTTGACTGGTC
AAGAGGGAGACTACGTTGTGCTTCCAAGTAACATCCAGGAGTTGTATGTTGATAATTGCAGAG
ACTTTCGTTGCCTATCTGATGTCTTGCTTCCTGTTGCATATCGTCATAGAAACTGCCAGAACGG
TGCAAGTGTAGACAGTTGCAACGGAGAGCAAGCTGAAGTTTTTGCAGGGCTTGAAATGTGCA
TTATCAGTAGATGCCAGGATGTTAAGACACTATTTGCACCAAGTTGGGTGGAGGAGAATCTCC
AATCCCTTGAATTATTACAAGTTGAAAATTGTAGACAAGTGAAGGAGATAATTGCAGAGGAAG
TTGTGGAATGTGGTCATTCTGAAGGAAAGGATGCTGTCATTAGCCTACCCCAACTTACGCGGC
TAGTTCTGACTCTGTTGCCTCAAGTGCATAGTGTCTATAAAGGTCTGCTAGTTTGTTCTTCACT
TCTGTGGTGTACTITTATGGACTGTCCAAAACTGAAGCGCCTTCCGTTCTCCAACATGGATCGT
CTAGAATGGATTGAAGGTCAAGAAAAATGGTGGGACTTGTTGGAATGGGATGAACCGGAATC
CAAAGCTTCGCTCAGACCCTTGTTTAGATGCAAGTCTGTGGATGGTGATCGATAACTGACTTG
ACCATCTTGCTGTGCTGGAGCTTGCTAGCTAGCTGTTTCAGAGTCCGAGATAAAACTTGTGTT
AAATGGTTCGCCTGCTTCCCTTCTCTTCCCCCAACCCCAACCCCAACCCCAACCCCAACCTCAC
CCTCACCCTTGCATTTCTTTTTTCAAATATACTTAGAAAAATAAAGAACAGGTAGAAGCCCATG
GTTGGCTTCCCAAATGTCAATCCTTCCTATTTCCTAAGATCAATTGTAGAGATGGATAACATCA
TCAAATATCGTATTAATCTTGTATTAATCTTGATAACGGATTCATATATGTTTTTATATAATAAAG
AATGATTATAATTGAGGCGAAAATAGAAATTTACGCTGTACGTAGTACTACATTGCAACCCAAA
AATTCACGCCACAAATTTTCGTTACTATTTACATTATTAATTCAGTTTAATGTCATAAAGGATAT
TATTGCATACTCTGTATAAGCTTCAATCATTGTTTATTAAACCACATATTACTTCATTTGTGTTTA
CTATTTATATGGACAAAGTTTTTAAATTTTGACCATATATGATAGTTAGATAATAGAAAAGGTTA
TTATCTTGTTGGATCAACATTGGATCCGTTTCAATAAATAAAAATTTCAACTAGTTCGTATCACT
TTTTTCTATCAGGGCCAACACTCGTAGCGGGAAAAATAATCTAGCGTGTTTGGAGGACTTTTG
GAAAGATATGGATTGGAATGAAGTTCATTTTATTTCAATTTCAATTTCAATGTTTGGTTAGGCAT
TTTAGATATATATGGAGTACTTGTGAAGAAATATGACGAGGAACACGTACGGTTGAGTTATGG
TTAAGTTCCTGCCCACATATTTGATATTTCTGGTTGAGTTCCTGTTATTCTTTACACTGCGATAA
AGCCCTCACATGTGACATAAAGCCACTACCTTGGCAGCCTCATTTTGTCTGCTAGTCAGTTTTG
AGAGTAATGCACCACAGTTGAGAAATGAACCGAGTGATTTTGAATGTTCCTTGCCTTGGTGGT
TATTGTTATATAAGCTCGTAAGTTTGTTATCAGGGGCACGGTAATACGGTATGATGAAAGAAG
ACATTCCATATGTTATCTTTTCCGCAACAATATAAACAGCATTCTAAATTCGAATGGTGCAAGA
ATTTATTACTTGACTGATAAAATCCAGCATGATCTCGGTAGATCAGCAACTTACAACGTAACTT
GAACAAATACGAAGTAAACTTTGAATAAATAAATGATGCTTGCTAGCTTCAATTCCATGGCAAA
TCGCGATTAGTTGCTTACTGGTGAGCTCCGTCTTTCAGCACAGAAATGAACGTCAACCAGACC
TGTTATACCAGCAGTTATCTTTTGTCCGGGTTTCACAGGCCCGATACCTTCTGGGGTGCCTGC
ACAAGACAAAAAACACATCAGAGTCTGGGCTATCAAGTTTACACACACAGAAACAGCAAAGTC
TAACAAGTAGTCTGTACTGTACCTGTTAGGATGACATCTCCTTCCATAAGTGTCATGAAGGAAC
TGATGTGGCTAATCAGGAATGGGATTTTGAATATCATGTCTTTTGTCGATCCCTTCTGCTTCGA
TTCTCCATCAACCTTTTTGTCATATAATTAAACTTGTTTACATTTGTATTAATACAATCACAAACA
ATTACATAGAATGTATTTACAGAGTTGAACTCACATCCAACCACAGCTCCAAGTTATGAGGATC
AGGCACTGCAGATACTGGAATCTGCAGAAAATTGACAAACCAGGGATCGGATAAATAATCCAG
CCATATACTGTACTGGACTCCTAATTATTTGTCGAATTCGACACAACTACATGAATTATACAAAT
CATATCATACCATTTCACTGATAGGGGTAAACGTGTCGAGTCCTTTAGCTAGTAGCCATGGAA
GACCTGTTGCCTAACAGAAGGAAAGGAGAAATTGTGAAACAGAATGTCAGGGGAGTATCTGA
AGTTTATTACATTATAGTTTTATAAATGGCAGAAACTGAATGTCAATACAAGTAAATTTCCACTG
AATGTACAAGTCAATGTTAAGAAGTATATGAACATATAGTCAAGATAATCACCTTAATTGTGGC
TTGGAGTTCCTTTGCTGTCATATCAAGTCCTAAAGCATAGCCTGAAAACTCAAAAACAATTTTT
TAAGTTGCTAATTATACTAAACTACTGAAGTTGTAACACACAGTACTTACTACAAGTAGTTCGT
GATTATGGAATAAGGGTGAACATTATTCTCATCCACCGGACCGGCCCGGCCCGGCCCTGATTA
CTCAATCGTAGTAAGAGTGGACATTACAGTGTAGGAAAGATGTCAACTTTTCCGTGCATTCTAT
AGATTAATTCTCAATTTCTCATTGCCATGAAAGAAACTCATAAAGAGTGACTTCTAACTAATTGT
TGTCCGTCCTTAGGGAGGATTTCTAAACCTCAAGTGACTTGTGTCCATATGAATACAAGTGAG
TACCAATACAGAAAATTTAGTACCAATACAAAATTTTAGTACCATTAACACAAAATTGCTTAGAA
ATATCAATACAAGTCATTTTGTGTTATTGGTACTAAAATATGATATATTGGTACTAAATTTTCTG
TATTGGTACTCGCTTGTGTTTATACACAAGTCACTTGAGGCCTAGACTTCCTCGTCCTCAGGCC
CTCCTCCCATACTCTAACATGTAGTCACGTGACTCACGTGTTTAAGCAATCATGAAGTCATGAG
GTGGTGAGGATGAGTTTGGAATCTAATATATATATATATATATATATAAAGGGGAGTTTTTTGG
AGAGATTTTAGAGCGTCCACGTAGGATTGTCCAGTCACATAATTAAATATTAATTTTATTITTAA
ATGATAAATAATGAGATAAAATTAAATATAATAGTAGTCAAATACTGATAAAAGACAATAAGGA
ATAATATTATCAGTAAAAGTATAGGATAATTATTATC
>SOV1g044450.1_coding sequence-
SEQ ID NO: 340
ATGGCGCATCAGATCCAGATTCCAATCAAGATGATTTCGATTATTGCTAATTTGTTTGGGATAG
GAAGGTATCACCGCGGTGTACCGTGTGATGGAGGAACTCTTGATAATCATCAATCTGAATTGA
GTAGTGTTCTTATACAACCTCAAAATAAACGAAGAAAAGTTGAGGAATTTCTTTGGTTTAATAC
TGAGTATGTTGATAGAATTATTGGTTGGTTAAAGGATAGTGAGATTGATACTATTGGTGTTTAT
GGGATGGGTGGAGTTGGGAAGACCACCCTCGCAACTCAGCTCCAAACTCGGCTATTGAATCA
TGTTGTTGCCTGGGTTTCTGTGGGGGTGGATTTTACTGTATATAAGTTGCAACAAGATATTGCC
GGGTCATTCGGGCTTGATCTTCAAGGGGATAGGGATGCGATTAGAAGAGCAGCTATGATTAG
TGAGTTTTTGTGTCGTAATCATAACTGTGTACTCTTCCTTGATGATTTATGGGGTGATTTCCGT
AGAGAAGATGTTGGAATTCCCAGAAAATGTAAGTTGATTGTCATTTCTCGGTTGCTGGATGTG
TGTCGTAAGCTTCGTTGCAAGAAGGTAGTTAAGGTTGAGCCACTTCAAGAGGAAGAGAGTTG
GCAGTTGTTTAACCGTAGCATAGGGTATGGTGTTTTGAATTCTGTAGATGTGTGTCGTATTAG
GAAGCTCGTGTATGATAAGTGTGTAGGTCTCCCTCTTGCTATCACTAGTTTAGCAACCAGTTTG
AGGAAGATGGTTGATGATGATAATCCTTCTAGTTGGAGGGAAGTTCTCGAGAATTTTGATCTT
CTATCTGCGAAACAACCTCACTTGGAAGATACATTTAGTCAGTTGAGGTTGTCATACGAAAGG
TTGAACAACAAACTCCAGCGTTGTTTCCTGTATTCTGCATTATTCCCCAAAGGCTATGCCATCA
GGAAAGAAGAGCTGATTCGTGTTTGGATTGGAATGGAGCTGATAGATGACGTGCCATCATTG
CAGGCACGGTATAACATGGGCCATTCTATATTGAATAAACTTGTTGACTCTTGTTTGCTGGAAA
CATATGATAATGATAACATAATCAAGATCAATGATCTTGTTAGGCATATGGCTCTTTCCATTGC
CGGAGATAGTTACATGGTAGAAGCTGGAATACCTACAAATTTTGAGTTCCGGGAAAATCTCCG
TGTTGTTTCCTTGGTGAATTCTTCAATACCATCGATCCCATCAGGTGCTTCCCTTATTAAGTGTC
ATTGTCTCTCTACTTTACTTCTGCAACACAACTCCCTTGAGTTTATCCCAGAGTCTTTTTTCTTG
CACATGCGTGGCCTTCGAGTTTTGAATCTATCTGATACGAACTTAATTAGACTGCCGAGTTCCA
TTGATGCCCTTGAAGAGCTTCGAGTCTTGGATCTTAGTTTCTGTCAAAACTTGAAGCAAGTGAC
ATCATTGTCAAAGCTTCTGAAATTACAGTTTCTAAACCTCTCTCAGACTGGAATTGGTCCAGCT
CCTCGTTCTCTTGAAAACTTAAAAAGTCTCACAGAACTCAACCTCTCATCTATTCCCGAACCAC
AAAAAATTCCTAGTGGTGTGTTGCCAGCCTTATTCAGACTCAAAAGATTAGCATGCTATGTTGT
AGAAGCTGCTATTCATGAGCTGCAGAATGTGAATTCATTAGAAGTTCTTGATGCCAGATTCTTA
AACCTTTGTGACCTGAGCAGTTATGTAAGGTCTCAGCATTGGTGCATATTAGAGAGCTATCAT
CTACAGGTTGGTTTTGAGGTGAGATCTGAGCAACCATACAACAGAAGAGTATCACTCTCCGGA
TGTTCTTTGACTGGTCAAGAGGGAGACTACGTTGTGCTTCCAAGTAACATCCAGGAGTTGTAT
GTTGATAATTGCAGAGACTTTCGTTGCCTATCTGATGTCTTGCTTCCTGTTGCATATCGTCATA
GAAACTGCCAGAACGGTGCAAGTGTAGACAGTTGCAACGGAGAGCAAGCTGAAGTTTTTGCA
GGGCTTGAAATGTGCATTATCAGTAGATGCCAGGATGTTAAGACACTATTTGCACCAAGTTGG
GTGGAGGAGAATCTCCAATCCCTTGAATTATTACAAGTTGAAAATTGTAGACAAGTGAAGGAG
ATAATTGCAGAGGAAGTTGTGGAATGTGGTCATTCTGAAGGAAAGGATGCTGTCATTAGCCTA
CCCCAACTTACGCGGCTAGTTCTGACTCTGTTGCCTCAAGTGCATAGTGTCTATAAAGGTCTG
CTAGTTTGTTCTTCACTTCTGTGGTGTACTTTTATGGACTGTCCAAAACTGAAGCGCCTTCCGT
TCTCCAACATGGATCGTCTAGAATGGATTGAAGGTCAAGAAAAATGGTGGGACTTGTTGGAAT
GGGATGAACCGGAATCCAAAGCTTCGCTCAGACCCTTGTTTAGATGCAAGTCTGTGGATGGTG
ATCGATAA
>SOV1g044450.1_protein-
SEQ ID NO: 341
MAHQIQIPIKMISIIANLFGIGRYHRGVPCDGGTLDNHQSELSSVLIQPQNKRRKVEEFLWFNTEY
VDRIIGWLKDSEIDTIGVYGMGGVGKTTLATQLQTRLLNHVVAWVSVGVDFTVYKLQQDIAGSF
GLDLQGDRDAIRRAAMISEFLCRNHNCVLFLDDLWGDFRREDVGIPRKCKLIVISRLLDVCRKLR
CKKVVKVEPLQEEESWQLFNRSIGYGVLNSVDVCRIRKLVYDKCVGLPLAITSLATSLRKMVDDD
NPSSWREVLENFDLLSAKQPHLEDTFSQLRLSYERLNNKLQRCFLYSALFPKGYAIRKEELIRVWI
GMELIDDVPSLQARYNMGHSILNKLVDSCLLETYDNDNIIKINDLVRHMALSIAGDSYMVEAGIPT
NFEFRENLRVVSLVNSSIPSIPSGASLIKCHCLSTLLLQHNSLEFIPESFFLHMRGLRVLNLSDTNLI
RLPSSIDALEELRVLDLSFCQNLKQVTSLSKLLKLQFLNLSQTGIGPAPRSLENLKSLTELNLSSIPE
PQKIPSGVLPALFRLKRLACYVVEAAIHELQNVNSLEVLDARFLNLCDLSSYVRSQHWCILESYHL
QVGFEVRSEQPYNRRVSLSGCSLTGQEGDYVVLPSNIQELYVDNCRDFRCLSDVLLPVAYRHRNC
QNGASVDSCNGEQAEVFAGLEMCIISRCQDVKTLFAPSWVEENLQSLELLQVENCRQVKEIIAEE
VVECGHSEGKDAVISLPQLTRLVLTLLPQVHSVYKGLLVCSSLLWCTFMDCPKLKRLPFSNMDRLE
WIEGQEKWWDLLEWDEPESKASLRPLFRCKSVDGDR*
SOV1g044450-cds-deletion-1-
SEQ ID NO: 342
MAHQIQIPIKMISIIANLFGIGRYHRGVPCDGGTLDNHQSELSSVLIQPQNKRRKVEEFLWFNTEY
VDRIIGWLKDSEIDTIGVYGMGGVGKTTLATQLQTRLLNHVVAWVSVGVDFTVYKLQQDIAGSF
GLDLQGDRDAIRRAAMISEFLCRNHNCVLFLDDLWGDFRREDVGIPRKCKLIVISRLLDVCRKLR
CKKVVKVEPLQEEESWQLFNRSIGYGVLNSVDVCRIRKLVYDKCVGLPLAITSLATSLRKMVDDD
NPSSWREVLENFDLLSAKQPHLEDTFSQLRLSYERLNNKLQRCFLYSALFPKGYAIRKEELIRVWI
GMELIDDVPSLQARYNMGHSILNKLVDSCLLETYDNDNIIKINDLVRHMALSIAGDSYMVEAGIPT
NFEFRENLRVVSLVNSSIPSIPSGASLIKCHCLSTLLLQHNSLEFIPESFFLHMRGLRVLNLSDTNLI
RLPSSIDALEELRVLDLSFCQNLKQVTSLSNLC┌ ┐ELQNVNSLEVLDARFLNLCDLSSYVRSQHWC
ILESYHLQVGFEVRSEQPYNRRVSLSRCSLTGQEGDYVVLPSNIQELYVDNCRDFRCLSDVLLPVA
YRHRNCQNGASVDSCNGEQAEVFAGLEMCIISRCQDVKTLFAPSWVEENLQSLELLQVENCRQV
KEIIAEEVVECGHSEGKDAVISLPQLTRLVLTLLPQVHSVYKGLLVCSSLLWCTFMDCPKLKRLPFS
NMDRLEWIEGQEKWWDLLEWDEPESKASLRPLFRCKSVDGDR*

### Example 4: RNA-Seq Analysis on the EMS22-20 Mutant Transcriptome Data

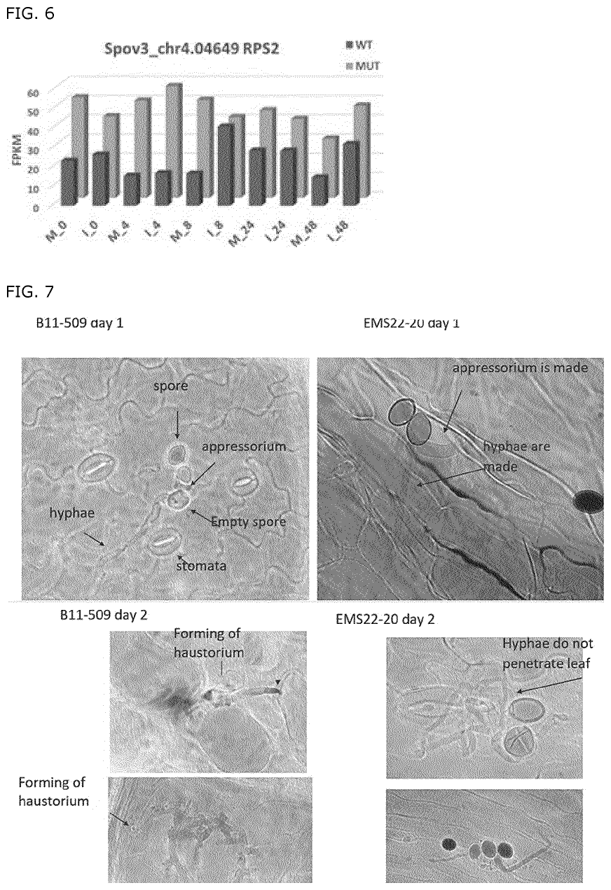

To get some insight into the resistance mechanism of the EMS22 mutant transcriptome data were obtained by performing an RNA-seq analysis. For the experiment, B11-509 and EMS22-20 were either mock-infected or infected with Pe:16 using a concentration of 1×10⁵ spores/ml. At different time points after infection (0, 4, 8, 24, and 48 hours post inoculation [hpi]) leaf samples were taken from at least three plants per treatment. RNA was isolated. In total, 20 equimolar bulks were analyzed. An RNA-seq data file of FPKM values was obtained. The RPS2-Like gene (SOV19044450) showed differential expression, with a higher expression in the EMS22 mutant than in the B11-509 background (FIG. 6). Visual inspection of two mock and treated pools revealed the same changes in mapping data of this gene. The RNA-seq results of B11-509 and EMS22-20 were used to analyze the expression level of spinach orthologs of known defense pathways (For example, SA, JA/Et, ABA, MAPK). This research shows that most of these orthologs are differentially expressed in the mutant. Our research thus indicates that said mutant shows a heightened level of basal resistance as many genes contributing to resistance (defense-related genes) are differentially expressed, such as PR genes (PR1-PR5b), chitinases, peroxidases, caffeate O-methyltransferases, aspartyl proteases, and MAPK3.

### Example 5: In Planta Resistance of EMS22-20 Line Against DM Isolate Pe:16

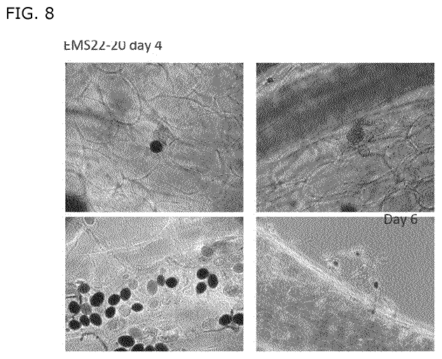

To get some insight into the resistance mechanism of the EMS22 mutant, detailed microscopic analyses were performed and the infection process of DM isolate Pe:16 on leaves of both B11-509 and EMS22-20 was followed over time using Trypan Blue staining. During the infection process in B11-509, penetration of the leaves by the appressoria and hyphae growth was already visible from day 2 onwards. In addition, it is believed that haustoria are formed. In case of the EMS22-20 mutant, it was clear that the downy mildew is not able to penetrate the leaf epidermis. Appressoria are formed, but the hyphae stay on the leaf surface, and often a cluster of spores and hyphae is observed (FIGS. 7 and 8).

### Example 6: In Planta Resistance of EMS22-20 Line Against Other Disease of Spinach

Diseases affecting spinach plants include, but are not limited to, anthracnose (Colletotrichum dematium f. sp. spinaciae), damping off/seedling blight (Pythium ultimum), downy mildew (Peronospora effusa), fusarium wilt (Fusarium oxysporum f. sp. spinaciae), Stemphylium leaf spot (Stemphylium versicarium, Stemphylium beticola, Stemphylium drummondii), verticillium wilt (Verticillium dahlia), white rust (Albugo occidentalis), black root rot (Aphanomyces cochlioides), and Cladosporium leaf spot (Cladosporium variabile). In order to determine if the resistance observed in EMS22-20 is broadly effective against other pathogens of spinach several disease assays are performed. Disease symptoms are evaluated on a scale from 1 (very susceptible/dead plant) to 9 (very resistant, no symptoms) for the lines carrying the homozygous causal mutations in RPS2-like gene (derived from EMS22-20) and other lines with the wild-type allele (including the untreated B11-509). Table 7 shows that the EMS-derived mutant allele of RPS2-like also provides increased resistance to Colletotrichum dematium f. sp. spinaciae, the latter causing antracnose on spinach, indicating that the resistance identified in EMS22-20 has a broad resistance activity beyond DM. The average disease score (24 plants per line) for plants carrying the resistant RPS2-like allele is “7 or 8”, compared to “3 or lower” in the B11-509, Viroflay and two additional susceptible varieties. Interestingly, the resistance score was also higher than that of Stanton (scored “6”) which is considered the state-of-the-art resistant variety for antracnose resistance in spinach. This research suggests that the EMS22-20 mutant S-allele results in a broad resistance against multiple pathogens and diseases common to spinach, and leads to the hypothesis that a similar resistance against further pathogens and/or diseases is to be expected.

TABLE 7
Results of an antracnose disease assay on spinach. 24 plants per line were infected
with Colletotrichum dematium f. sp. spinaciae and evaluated on a scale from
1 (very susceptible) to 9 (completely resistant). Lines carrying the mutant allele
of the S-locus (X20-010-1) were highly resistant compared to all other lines,
including compared to the best available variety (Stanton) in the market.
The homozygous presence of the mutated S-locus in the various lines is
indicated ‘Y’ = present while ‘N’ = absent. ‘Susceptible control line’
indicates that the line is known to be susceptible against Colletotrichum
(negative control), while ‘Resistant control line’ indicates that the
line is known to be resistant against Colletotrichum (positive control).

| | Homozygous | | |
|---|---|---|---|
| | presence of | Disease score (1- | |
| Genotype/line | mutated S-locus | 9) Colletotrichum | Comment |
| PV1028 | N | 2 | Susceptible control line |
| PV1369 | N | 3 | Susceptible control line |
| Stanton | N | 6 | Resistant control line |
| Viroflay | N | 3 | Susceptible control line |
| B11-509 | N | 3 | Susceptible control line |
| X20-010-1-12-5 | Y | 8 | |
| X20-010-1-13-2 | Y | 7 | |
| X20-010-1-13-3 | Y | 8 | |
| X20-010-1-13-6 | Y | 8 | |
| X20-010-1-13-7 | Y | 8 | |
| X20-010-1-13-13 | Y | 8 | |

### Example 7: In Planta Functional Validation of RPS2-Like Causing Resistance of EMS22-20 Line Against DM Isolate Pe:16

To analyze gain and loss of resistance, a transient expression assay was set up, as stable transformation in spinach was not achievable. Full length coding sequences of the candidate genes, both wild-type (WT) and mutant alleles, were cloned into overexpression (OE) construct pK7WG2, under the control of the 35S promoter. To check for expression levels in inoculated tissues, leaves and cotyledons of wild-type B11-509 and mutant EMS22-20 were inoculated with A. tumefaciens containing the OE constructs. Leaves from 3- or 4-week-old plants and cotyledons of 10-days old seedlings were used for transient expression of the candidate gens (via A. tumefaciens) and subsequently inoculated with Pe:16 for functional validation of the candidate genes. It is hypothesized that OE of the WT allele of RSP2-like in the EMS22-20 background (complementation) will restore the susceptibility of the EMS22-20 mutant to P. effusa, whereas OE of the mutant allele does not.

### Example 8: Syntenic Analysis of Spo10764 and Spov3_Chr4.04752 in Pea, Soy and Faba Bean

Spo10764 and Spov3_chr4.04752 (Sp75 gene model ID Spo25330) are candidate genes in spinach associated with downy mildew susceptibility.

Spo10764 is found on chromosome 1 (41,268,940-41,270,931) of the Sp75 pseudomoleculed reference genome (MO ID: 27091) and consists of a single exon.

Spov3_chr4.04752 is found on chromosome 1 (50,406,246-50,414,563) of the Sp75 pseudomoleculed reference genome (MO ID: 27091) and consists of 11 exons.

### Orthologues in Pea, Soy and Faba Bean

Genomic, CDS and protein sequences were provided for the two candidate genes. A preliminary investigation into potential orthologues was carried out with a simplistic BLAST approach. This was then followed up using the Synteny Analyser (available on galaxy-test.kws.de), which mainly utilises JCVI and was designed for analysis of cereals, but for the proposes of this task, was updated to include a wider range of crops.

### Pea-Spo10764

Reference genome=cv. Caméor v1 (MO ID: 28707)

BLAST results:

| | Coverage | | |
|---|---|---|---|
| Chr/Gene/Protein | (%) | ID (%) | BLAST type |
| KE_Cameor_v1.scaffold03758 | 53 | 66 | CDS −> Genomic |
| Psat0s3758g0080.1 | 53 | 66 | CDS −> CDS |
| KE_Cameor_v1.scaffold03758 | 53 | 66 | Genomic −> Genomic |
| Psat0s3758g0080.1 | 53 | 66 | Genomic −> CDS |
| Psat0s3758g0080.1 | 92 | 37 | Protein −> Protein |

Psat0s3758g0080 is found on scaffold_03758 of the pea genome and is the best fit from the pea gene model to Spo10764.

### Soy-Spo10764

Reference genome=cv. Williams82 v4 (MO ID: 28479)
BLAST results:

| | Coverage | | |
|---|---|---|---|
| Chr/Gene/Protein | (%) | ID (%) | BLAST type |
| Glyma.08G285350.1 | 54 | 67 | CDS −> CDS |
| Glyma.08G285300.2 | 54 | 67 | CDS −> CDS |
| SOY_Wm82_v4.chr08 | 54 | 66 | CDS −> Genomic |
| SOY_Wm82_v4.chr18 | 53 | 66 | CDS −> Genomic |
| Glyma.08G285350.1 | 54 | 67 | Genomic −> CDS |
| Glyma.08G285300.2 | 54 | 67 | Genomic −> CDS |
| SOY_Wm82_v4.chr08 | 54 | 66 | Genomic −> Genomic |
| SOY_Wm82_v4.chr18 | 47 | 66 | Genomic −> Genomic |
| ***no hits*** | | | Protein −> Protein |

Glyma.08G285350.1 and Glyma.08G285300.2 are both found on chromosome 8 of the soy genome and are the best fit from the soy gene model to Spo10764. The Synteny Analyser also identifies the orthologue for Spo10764 as Glyma.08G285300.2 in soy cv. Williams82, with default filter settings (syntenic blocks consisting of a minimum of 30 genes).

### Pea-Spov3_chr4.04752

Reference genome=cv. Caméor v1 (MO ID: 28707)
BLAST results:

| | Coverage | | |
|---|---|---|---|
| Chr/Gene/Protein | (%) | ID (%) | BLAST type |
| Psat4g115080.1 | 100 | 81 | CDS −> CDS |
| Psat2g093960.1 | 99 | 80 | CDS −> CDS |
| KE_Cameor_v1.chr4 | 26 | 72 | Genomic −> Genomic |
| KE_Cameor_v1.chr2 | 22 | 73 | Genomic −> Genomic |
| Psat4g115080.1 | 100 | 92 | Protein −> Protein |
| Psat2g093960.1 | 100 | 91 | Protein −> Protein |

Psat4g115080.1 and Psat2g093960.1 are found on chromosome 2 and 4 respectively and are the best fit from the pea gene model to Spov3_chr4.04752. Psat4g115080.1 and Psat2g093960.1 consist of 11 exons, which is consistent with the Spov3_chr4.04752 (Spo25330) gene model. The Synteny Analyser found Psat4g115080.1 to be a syntenic block with Spov3_chr4.04752.

### Soy-Spov3_chr4.04752

Reference genome=cv. Williams82 v4 (MO ID: 28479)
BLAST results:

| | Coverage | | |
|---|---|---|---|
| Chr/Gene/Protein | (%) | ID (%) | BLAST type |
| Glyma.18G143100.1 | 100 | 81 | CDS −> CDS |
| Glyma.08G283400.1 | 100 | 80 | CDS −> CDS |
| Glyma.01G027300.1 | 99 | 79 | CDS −> CDS |
| Glyma.02G037700.1 | 99 | 79 | CDS −> CDS |
| SOY_Wm82_v4.chr18 | 23 | 71 | Genomic −> Genomic |
| SOY_Wm82_v4.chr08 | 21 | 74 | Genomic −> Genomic |
| SOY_Wm82_v4.chr02 | 25 | 69 | Genomic −> Genomic |
| SOY_Wm82_v4.chr01 | 20 | 72 | Genomic −> Genomic |

Glyma.18G143100.1, Glyma.08G283400.1, Glyma.01G027300.1 and Glyma.02G037700.1 are found on chromosomes 18, 8, 1 and 2, respectively. These all consist of 11 exons, which is consistent with the Spov3_chr4.04752 (Spo25330) gene model. The Synteny Analyser identified all four gene models (Glyma.18G143100.1, Glyma.08G283400.1, Glyma.01G027300.1 and Glyma.02G037700.1) as orthologues found in syntenic blocks with Spov3_chr4.04752.

### Faba Bean-Spov3_chr4.04752

Reference transcriptome=V.faba_csf_roftrans v2-available at pulsedb.org

Faba bean currently has no genome available, but instead has a transcriptome of assembled RNASeq data. Therefore, it is suitable for BLAST analysis but not compatible with the Synteny Analyser.

| | Coverage | | |
|---|---|---|---|
| Chr/Gene/Protein | (%) | ID (%) | BLAST type |
| v.faba_CSFL_reftransV2_0011954 | 48 | 82 | CDS −> CDS |

Spov3_chr4.04752 returned a single hit in the faba bean transcriptome.

TABLE 8

| Candidate | | | | | |
|---|---|---|---|---|---|
| Gene | Genome | Gene model | Genomic | CDS | Protein |
| Spo10764 | Soybean | Glyma.08G285300.2 | SEQ ID NO: 316 | SEQ ID NO: 321 | SEQ ID NO: 326 |
| Spov3_chr4.04752 | Pea | Psat4g115080.1 | SEQ ID NO: 252 | SEQ ID NO: 271 | SEQ ID NO: 294 |
| | | Psat2g093960.1 | SEQ ID NO: 253 | SEQ ID NO: 272 | SEQ ID NO: 295 |
| | Soybean | Glyma.18G143100.1 | SEQ ID NO: 254 | SEQ ID NO: 273 | SEQ ID NO: 296 |
| | | Glyma.08G283400.1 | SEQ ID NO: 255 | SEQ ID NO: 274 | SEQ ID NO: 297 |
| | | Glyma.01G027300.1 | SEQ ID NO: 256 | SEQ ID NO: 275 | SEQ ID NO: 298 |
| | | Glyma.02G037700.1 | SEQ ID NO: 257 | SEQ ID NO: 276 | SEQ ID NO: 299 |
| | Faba | V.faba_CSFL_reftransV2_0011954 | — | SEQ ID NO: 277 | — |
| | bean | | | | |

### Example 9: Downy Mildew Resistance in Sunflower Plants

It is hypothesized that sunflower plants with altered functionality of the susceptibility gene (orthologous to SEQ ID NO: 4, 96, 172) or protein (orthologous to SEQ ID NO: 6, 98, or 174) will be less susceptible to downy mildew infection. In order to test this hypothesis, sunflower mutant plants altered in the expression of the S-gene (and translation of the protein) will be generated through gene editing technologies or via chemical mutagenesis. Subsequently, these mutants, homozygous for the mutant allele, will be tested in a bioassay to determine their level of reduced susceptibility towards downy mildew infection. The results will be compared to mutant plants that only have one allele mutated or plants with the un-mutated allele. More specifically, the bioassays will be conducted on seedlings of sunflower. The plants will be directly inoculated with one or more races (strains or isolates) of the pathogen Plasmopara halstedii. Alternatively, seeds will be sown, or seedlings will be transferred to potting soil that is mixed with infectious-soil (soil known to cause disease in susceptible plants). Level of susceptibility will be assessed in numerous ways. For example, by determining the number of germinating seedlings after sowing or scoring the percentage of diseased seedlings, determining the level of infection (disease class scoring) or the presence of the pathogen by diagnostic assays. In all of these assays, sunflower plants homozygous for the mutant allele will be compared to plants that have only one mutant allele (heterozygous) or only carry the un-mutated allele (homozygous wild-type). It will be demonstrated that sunflower plants homozygous for the mutant allele are less-susceptible to downy mildew compared to plants that carry the un-mutated (wild-type) variant.

### Example 10: Downy Mildew Resistance in Maize Plants

It is hypothesized that maize plants with altered functionality of the susceptibility gene (orthologous to SEQ ID NO: 4, 96, 172) or protein (orthologous to SEQ ID NO: 6, 98, or 174) will be less susceptible to downy mildew infection. In order to test this hypothesis, maize mutant plants altered in the expression of the S-gene (and translation of the protein) will be generated through gene editing technologies or via chemical mutagenesis. Subsequently, these mutants, homozygous for the mutant allele, will be tested in a bioassay to determine their level of reduced susceptibility towards downy mildew infection. The results will be compared to mutant plants that only have one allele mutated or plants with the un-mutated allele. More specifically, the bioassays will be conducted on seedlings, detached leaves or leaf discs of maize. The plants will be directly inoculated by rub inoculation or sprayed with an inoculum suspension with sporangia/spores with one or more races (strains or isolates) of the pathogen Peronosclerospora spp (e.g. sorghi, maydis, sacchari). Level of susceptibility will be assessed in numerous ways. For example by determining the percentage of diseased seedlings, determining the level of infection (disease class scoring) or the presence of the pathogen by diagnostic assays. In all of these assays, maize plants homozygous for the mutant allele will be compared to plants that have only one mutant allele (heterozygous) or only carry the un-mutated allele (homozygous wild-type). It will be demonstrated that maize plants homozygous for the mutant allele are less-susceptible to downy mildew compared to plants that carry the un-mutated (wild-type) variant.

### Example 11: Downy Mildew Resistance in Beta vulgaris Plants

It is hypothesized that sugar beet, red beet and Swiss chard (all Beta vulgaris species) plants with altered functionality of the susceptibility gene (orthologous to SEQ ID NO: 4, 96, 172) or protein (orthologous to SEQ ID NO: 6, 98, or 174) will be less susceptible to downy mildew infection. In order to test this hypothesis, Beta vulgaris mutant plants altered in the expression of the S-gene (and translation of the protein) will be generated through gene editing technologies or via chemical mutagenesis. Subsequently, these mutants, homozygous for the mutant allele, will be tested in a bioassay to determine their level of reduced susceptibility towards downy mildew infection. The results will be compared to mutant plants that only have one allele mutated or plants with the un-mutated allele. More specifically, the bioassays will be conducted on seedlings, detached leaves or leaf discs of Beta vulgaris. The plants will be directly inoculated by rub inoculation or sprayed with an inoculum suspension with sporangia/spores with one or more races (strains or isolates) of the pathogen Peronospora farinosa f. sp. betae. Level of susceptibility will be assessed in numerous ways. For example by determining the percentage of diseased seedlings, determining the level of infection (disease class scoring) or the presence of the pathogen by diagnostic assays. In all of these assays, Beta vulgaris plants homozygous for the mutant allele will be compared to plants that have only one mutant allele (heterozygous) or only carry the un-mutated allele (homozygous wild-type). It will be demonstrated that Beta vulgaris plants homozygous for the mutant allele are less-susceptible to downy mildew compared to plants that carry the un-mutated (wild-type) variant.

### Example 12: Downy Mildew Resistance in Lettuce Plants

It is hypothesized that lettuce plants with altered functionality of the susceptibility gene (orthologous to SEQ ID NO: 4, 96, 172) or protein (orthologous to SEQ ID NO: 6, 98, or 174) will be less susceptible to downy mildew infection. In order to test this hypothesis, lettuce mutant plants altered in the expression of the S-gene (and translation of the protein) will be generated through gene editing technologies or via chemical mutagenesis. Subsequently, these mutants, homozygous for the mutant allele, will be tested in a bioassay to determine their level of reduced susceptibility towards downy mildew infection. The results will be compared to mutant plants that only have one allele mutated or plants with the un-mutated allele. More specifically, the bioassays will be conducted on seedlings, detached leaves or leaf discs of lettuce. The plants will be directly inoculated by rub inoculation or sprayed with an inoculum suspension with sporangia/spores with one or more races (strains or isolates) of the pathogen Bremia lactucae. Level of susceptibility will be assessed in numerous ways. For example by determining the percentage of diseased seedlings, determining the level of infection (disease class scoring) or the presence of the pathogen by diagnostic assays. In all of these assays, lettuce plants homozygous for the mutant allele will be compared to plants that have only one mutant allele (heterozygous) or only carry the un-mutated allele (homozygous wild-type). It will be demonstrated that lettuce plants homozygous for the mutant allele are less-susceptible to downy mildew compared to plants that carry the un-mutated (wild-type) variant.

### Example 13: Downy Mildew Resistance in Plants of the Family Cucurbitaceae

It is hypothesized that plants in the family of the Cucurbitaceae (e.g., cucumber, melon, watermelon, squash, pumpkin, gourd and others crops) with altered functionality of the susceptibility gene (orthologous to SEQ ID NO: 4, 96, 172) or protein (orthologous to SEQ ID NO: 6, 98, or 174) will be less susceptible to downy mildew infection. In order to test this hypothesis, mutant plants altered in the expression of the S-gene (and translation of the protein) will be generated through gene editing technologies or via chemical mutagenesis. Subsequently, these mutants, homozygous for the mutant allele, will be tested in a bioassay to determine their level of reduced susceptibility towards downy mildew infection. The results will be compared to mutant plants that only have one allele mutated or plants with the un-mutated allele. More specifically, the bioassays will be conducted on seedlings, detached leaves or leaf discs. The plants will be directly inoculated by rub inoculation or sprayed with an inoculum suspension with sporangia/spores with one or more races (strains or isolates) of the pathogen Pseudoperonospora cubensis. Level of susceptibility will be assesses in numerous ways. For example by determining the percentage of diseased seedlings, determining the level of infection (disease class scoring) or the presence of the pathogen by diagnostic assays. In all of these assays, plants homozygous for the mutant allele will be compared to plants that have only one mutant allele (heterozygous) or only carry the un-mutated allele (homozygous wild-type). It will be demonstrated that plants homozygous for the mutant allele are less-susceptible to downy mildew compared to plants that carry the un-mutated (wild-type) variant.

### Example 14: Downy Mildew Resistance in Cucumber Plants

It is hypothesized that cucumber plants with altered functionality of the susceptibility gene (orthologous to SEQ ID NO: 4, 96, 172) or protein (orthologous to SEQ ID NO: 6, 98, or 174) will be less susceptible to downy mildew infection. In order to test this hypothesis, cucumber mutant plants altered in the expression of the S-gene (and translation of the protein) will be generated through gene editing technologies or via chemical mutagenesis. Subsequently, these mutants, homozygous for the mutant allele, will be tested in a bioassay to determine their level of reduced susceptibility towards downy mildew infection. The results will be compared to mutant plants that only have one allele mutated or plants with the un-mutated allele. More specifically, the bioassays will be conducted on seedlings, detached leaves or leaf discs of cucumber. The plants will be directly inoculated by rub inoculation or sprayed with an inoculum suspension with sporangia/spores with one or more races (strains or isolates) of the pathogen Pseudoperonospora cubensis. Level of susceptibility will be assessed in numerous ways. For example by determining the percentage of diseased seedlings, determining the level of infection (disease class scoring) or the presence of the pathogen by diagnostic assays. In all of these assays, cucumber plants homozygous for the mutant allele will be compared to plants that have only one mutant allele (heterozygous) or only carry the un-mutated allele (homozygous wild-type). It will be demonstrated that cucumber plants homozygous for the mutant allele are less-susceptible to downy mildew compared to plants that carry the un-mutated (wild-type) variant.

### Example 15: Downy Mildew Resistance in Additional Plant Species

It is hypothesized that additional plants, such as but not limited to, carrot, bean, rocket (arugula), basil, soybean, sorghum, impatiens, wheat, barley, rye, pea, faba (fava) bean, plants of the Brassica species (such as, but not limited to, Brassica napus, Brassica oleracea, Brassica rapa, and Brassica juncea), and plants of the Family of Solanaceae (such as, but not limited to, tomato, pepper, and eggplant) with altered functionality of the susceptibility gene (orthologous to SEQ ID NO: 4, 96, 172) or protein (orthologous to SEQ ID NO: 6, 98, or 174) will be less susceptible to downy mildew infection. In order to test this hypothesis, mutant plants which are altered in the expression of the S-gene (and translation of the protein) will be generated through gene editing technologies or via chemical mutagenesis. Subsequently, these mutants, homozygous for the mutant allele, will be tested in a bioassay to determine their level of reduced susceptibility towards downy mildew infection. The results will be compared to mutant plants that only have one allele mutated or plants with the un-mutated allele. More specifically, the bioassays will be conducted on seedlings, detached leaves or leaf discs. The plants will be directly inoculated by rub inoculation or sprayed with an inoculum suspension with sporangia/spores with one or more races (strains or isolates) of pathogen. The following crop/downy mildew combinations will be tested: carrot/Peronospora parasitica; rocket (arugula)/Peronospora erucastri; belbahrii; soybean/Peronospora manshurica; basil/Peronospora sorghum/Peronosclerospora sorghi; impatiens/Plasmopara obducen: wheat/Sclerophthora macrospora; barley/Sclerophthora macrospora; rye/Sclerophthora macrospora; pea/Phytophthora phaseoli; faba (fava) bean/Phytophthora phaseoli; Brassica species/Hyaloperonospora brassicae; and Family of Solanaceaea/Peronospora hyoscyami. Level of susceptibility will be assessed in numerous ways. For example by determining the percentage of diseased seedlings, determining the level of infection (disease class scoring) or the presence of the pathogen by diagnostic assays. In all of these assays, plants homozygous for the mutant allele will be compared to plants that have only one mutant allele (heterozygous) or only carry the un-mutated allele (homozygous wild-type). It will be demonstrated that plants homozygous for the mutant allele are less-susceptible to downy mildew compared to plants that carry the un-mutated (wild-type) variant.

### Example 16: Resistance to Phytophthora Blight Diseases that Cause Root, Crown and Fruit Rots

Reduced susceptibility to other oomycete pathogens, most importantly Phytophthora spp. will be determined in a similar manner. Plants homozygous for the mutant allele will be compared to plants that have only one mutant allele (heterozygous) or only carry the un-mutated allele (homozygous wild-type). It will be demonstrated that plants homozygous for the mutant allele are less-susceptible to Phytophthora blight compared to plants that carry the un-mutated (wild-type) variant. The following crop/pathogen combinations will be tested: tomato/Phytophthora infestans (late blight); tomato/Phytophthora capsici; pepper/Phytophthora capsici; eggplant/Phytophthora capsici; tobacco/Phytophthora nicotianae; and cucurbit crops (e.g., cucumber, squash, pumpkin, watermelon, and melon)/Phytophthora capsica.

### Example 17: Resistance to Pythium Diseases that Cause Damping-Off and Root-Rot

Reduced susceptibility to other oomycete pathogens, most importantly Pythium spp. will be determined in a similar manner. Plants homozygous for the mutant allele will be compared to plants that have only one mutant allele (heterozygous) or only carry the un-mutated allele (homozygous wild-type). It will be demonstrated that plants homozygous for the mutant allele are less-susceptible to Pythium diseases compared to plants that carry the un-mutated (wild-type) variant. The following crop/pathogen combinations will be tested: carrot/Pythium irregulare, Pythium violae, and Pythium ultimum; Swiss chard/Pythium irregulare, Pythium violae, and Pythium ultimum; and spinach/Pythium irregulare, Pythium violae, and Pythium ultimum.

### Deposit Information

A total of 625 seeds of resistant M3 spinach line ‘Nr.22-18’ were deposited under accession number NCIMB 43773 by KWS Vegetables B.V. on May 14, 2021, at the NCIMB Ltd., Ferguson Building, Craibstone Estate, Bucksbum, Aberdeen AB21 9YA, United Kingdom (NCIMB). Access to the deposit will be available during the pendency of this application to persons determined by the Director of the U.S. Patent Office to be entitled thereto upon request or by the expert solution according to Rule 32 EPC. Subject to 37 C.F.R. § 1.808 (b), all restrictions imposed by the depositor on the availability to the public of the deposited material will be irrevocably removed upon the granting of the patent. The deposit will be maintained for a period of 30 years, or 5 years after the most recent request or for the enforceable life of the patent whichever is longer, and will be replaced if it ever becomes nonviable during that period. Applicant does not waive any rights granted under this patent on this application or under the Plant Variety Protection Act (7 USC 2321 et seq.).

1. A method of reducing susceptibility to a pathogen in a plant, the method comprising:
Increasing or decreasing the activity or expression of a susceptibility gene (S-gene) encoding an RPS2-like resistance protein.

2. The method of claim 1 wherein said increased or decreased activity is by targeted DNA modification or by introgression.

3. The method of claim 2, wherein said DNA modification results in a sequence selected from SEQ ID NOs: 169 or 170.

4. The method of claim 1, wherein the S-gene encodes an RPS2-like resistance protein comprising an amino acid sequence that is at least 80% identical to the sequence of SEQ ID NO: 174.

5. The method of claim 1, wherein the plant is of the species Spinacia oleracea.

6. The method of claim 1, wherein the pathogen is of the phylum Oomycota.

7. The method of claim 1, wherein the pathogen is of the phylum Ascomyceta.

8. The method of claim 2, wherein the modification comprises and is flanked by SEQ ID NO: 241 and SEQ ID NO: 251.

9. The method of claim 6, wherein the pathogen is of the species Peronospora effusa.

10. The method of claim 7, wherein the pathogen is of the genus Colletotrichum.

11. The method of claim 10, wherein the pathogen is of the species Colletotrichum dematium f. sp. spinaciae.

12. A plant with reduced susceptibility to a pathogen comprising one or more nucleotide modifications at a genomic locus of the plant, wherein the genomic locus comprises a modified susceptibility gene (S-gene) encoding an RPS2-like resistance protein when compared to a plant without the modification.

13. The plant of claim 12, wherein the one or more nucleotide modifications result in increased or decreased expression or activity of a protein encoded by the S-gene, optionally wherein the one or more nucleotide modifications comprise an insertion, a deletion, or a single nucleotide polymorphism (SNP).

14. The plant of claim 12, wherein the S-gene encodes an RPS2-like resistance protein comprising an amino acid sequence that is at least 80% identical to the sequence of SEQ ID NO: 174.

15. The plant of claim 12, wherein the one or more nucleotide modifications result in a sequence selected from SEQ ID NOs: 169 or 170.

16. The plant of claim 12, wherein the plant is of the species Spinacia oleracea, and the pathogen is of the species Peronospora effusa.

17. The plant of claim 12, wherein the plant is of the species Spinacia oleracea, and the pathogen is of the genus Colletotrichum, preferably of the species Colletotrichum dematium f. sp. spinaciae.

18. The plant of claim 12, wherein the genomic locus comprises and is flanked by SEQ ID NO: 241 and SEQ ID NO: 251.

19. A plant cell, a seed, a progeny plant, plant part, or plant variety from the plant of claim 12.

20-38. (canceled)
